(19) United States
(12) Patent Application Publication
(10) Pub. No.: US 2006/0276629 A9
(43) Pub. Date: Dec. 7, 2006

(54) Purification and characterization of soluble human HLA proteins

(76) Inventors: William H. Hildebrand, Edmond, OK (US); Rico Buchli, Edmond, OK (US)

Correspondence Address: DUNLAP, CODDING & ROGERS P.C., PO BOX 16370, OKLAHOMA CITY, OK, 73113, US

(21) Appl. No.: 10/337,161

(22) Filed: Jan. 2, 2003

Related U.S. Application Data
(63) Continuation-in-part of application No. 10/022,066, filed on Dec. 18, 2001, now abandoned.
(63) Continuation-in-part of application No. 09/929,852, filed on Aug. 14, 2001, now abandoned.
Application No. 09/929,852 is a continuation of application No. 09/465,321, filed on Dec. 17, 1999, now abandoned.
(60) Provisional application No. 60/347,906, filed on Jan. 2, 2002.

(51) Int. Cl. C07K 14/74

(52) U.S. Cl. 530/350

(57) ABSTRACT

The present invention relates generally to the production and use of functionally active soluble HLA molecules that are isolated and purified substantially away from other proteins, and methods of purifying same.

### CROSS-REFERENCE TO RELATED APPLICATIONS

This application claims the benefit under 35 U.S.C. 119(e) of provisional application U.S. Serial No. 60/347,906, filed Jan. 2, 2002, entitled “sHLA ASSAY METHODOLOGIES,” the contents of which are hereby expressly incorporated herein by reference in their entirety.

This application is also a continuation-in-part of U.S. Ser. No. 10/022,066, filed Dec. 18, 2001, entitled “METHOD AND APPARATUS FOR THE PRODUCTION OF SOLUBLE MHC ANTIGENS AND USES THEREOF,” the contents of which are hereby expressly incorporated herein by reference in their entirety.

### STATEMENT REGARDING FEDERALLY SPONSORED RESEARCH OR DEVELOPMENT

Not Applicable.

### BACKGROUND OF THE INVENTION

1. Field of the Invention

The present invention relates generally to the production and use of functionally active soluble HLA molecules that are isolated and purified substantially away from other proteins, and methods of purifying same.

2. Description of the Background Art

Class I major histocompatibility complex (MHC) molecules, designated HLA class I in humans, bind and display peptide antigen ligands upon the cell surface. The peptide antigen ligands presented by the class I MHC molecule are derived from either normal endogenous proteins (“self”) or foreign proteins (“nonself”) introduced into the cell. Nonself proteins may be products of malignant transformation or intracellular pathogens such as viruses. In this manner, class I MHC molecules convey information regarding the internal fitness of a cell to immune effector cells including but not limited to, CD8⁺ cytotoxic T lymphocytes (CTLs), which are activated upon interaction with “nonself” peptides, thereby lysing or killing the cell presenting such “nonself” peptides.

Class II MHC molecules, designated HLA class II in humans, also bind and display peptide antigen ligands upon the cell surface. Unlike class I MHC molecules which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptides they bind and present are derived from extracellular foreign antigens, such as products of bacteria that multiply outside of cells, wherein such products include protein toxins secreted by the bacteria that often times have deleterious and even lethal effects on the host (e.g. human). In this manner, class II molecules convey information regarding the fitness of the extracellular space in the vicinity of the cell displaying the class II molecule to immune effector cells, including but not limited to, CD4⁺ helper T cells, thereby helping to eliminate such pathogens. The examination of such pathogens is accomplished by both helping B cells make antibodies against microbes, as well as toxins produced by such microbes, and by activating macrophages to destroy ingested microbes.

Class I and class II HLA molecules exhibit extensive polymorphism generated by systematic recombinatorial and point mutation events; as such, hundreds of different HLA types exist throughout the world's population, resulting in a large immunological diversity. Such extensive HLA diversity throughout the population results in tissue or organ transplant rejection between individuals as well as differing susceptibilities and/or resistances to infectious diseases. HLA molecules also contribute significantly to autoimmunity and cancer. Because HLA molecules mediate most, if not all, adaptive immune responses, large quantities of pure isolated HLA proteins are required in order to effectively study transplantation, autoimmunity disorders, and for vaccine development.

There are several applications in which purified, individual class I and class II MHC proteins are highly useful. Such applications include using MHC-peptide multimers as immunodiagnostic reagents for disease resistance/autoimmunity; assessing the binding of potentially therapeutic peptides; elution of peptides from MHC molecules to identify vaccine candidates; screening transplant patients for preformed MHC specific antibodies; and removal of anti-HLA antibodies from a patient. Since every individual has differing MHC molecules, the testing of numerous individual MHC molecules is a prerequisite for understanding the differences in disease susceptibility between individuals. Therefore, isolated and purified MHC molecules that are representative of the hundreds of different HLA types existing throughout the world's population are highly desirable for unraveling disease susceptibilities and resistances, as well as for designing therapeutics such as vaccines.

Class I HLA molecules alert the immune response to disorders within host cells. Peptides, which are derived from viral- and tumor-specific proteins within the cell, are loaded into the class I molecule's antigen binding groove in the endoplasmic reticulum of the cell and subsequently carried to the cell surface. Once the class I HLA molecule and its loaded peptide ligand are on the cell surface, the class I molecule and its peptide ligand are accessible to cytotoxic T lymphocytes (CTL). CTL survey the peptides presented by the class I molecule and destroy those cells harboring ligands derived from infectious or neoplastic agents within that cell.

While specific CTL targets have been identified, little is known about the breadth and nature of ligands presented on the surface of a diseased cell. From a basic science perspective, many outstanding questions have permeated through the art regarding peptide exhibition. For instance, it has been demonstrated that a virus can preferentially block expression of HLA class I molecules from a given locus while leaving expression at other loci intact. Similarly, there are numerous reports of cancerous cells that fail to express class I HLA at particular loci. However, there is no data describing how (or if) the three classical HLA class I loci differ in the immunoregulatory ligands they bind. It is therefore unclear how class I molecules from the different loci vary in their interaction with viral- and tumor-derived ligands and the number of peptides each will present.

Discerning virus- and tumor-specific ligands for CTL recognition is an important component of vaccine design. Ligands unique to tumorigenic or infected cells can be tested and incorporated into vaccines designed to evoke a protective CTL response. Several methodologies are currently employed to identify potentially protective peptide ligands. One approach uses T cell lines or clones to screen for biologically active ligands among chromatographic fractions of eluted peptides (Cox et al., Science, vol 264, 1994, pages 716-719, which is expressly incorporated herein by reference in its entirety). This approach has been employed to identify peptides ligands specific to cancerous cells. A second technique utilizes predictive algorithms to identify peptides capable of binding to a particular class I molecule based upon previously determined motif and/or individual ligand sequences (De Groot et al., Emerging Infectious Diseases, (7) 4, 2001, which is expressly incorporated herein by reference in its entirety). Peptides having high predicted probability of binding from a pathogen of interest can then be synthesized and tested for T cell reactivity in precursor, tetramer or ELISpot assays.

However, prior to the presently claimed and disclosed invention(s) there has been no readily available source of individual isolated and purified HLA molecules. The quantities of HLA protein previously available have been small and typically consist of a mixture of different HLA molecules. Production of HLA molecules traditionally involves growth and lysis of cells expressing multiple HLA molecules. Ninety percent of the population is heterozygous at each of the HLA loci; codominant expression results in multiple HLA proteins expressed at each HLA locus. To purify native class I or class II molecules from mammalian cells requires time-consuming and cumbersome purification methods, and since each cell typically expresses multiple surface-bound HLA class I or class II molecules, HLA purification results in a mixture of many different HLA class I or class II molecules. When performing experiments using such a mixture of HLA molecules or performing experiments using a cell having multiple surface-bound HLA molecules, interpretation of results cannot directly distinguish between the different HLA molecules, and one cannot be certain that any particular HLA molecule is responsible for a given result. Therefore, prior to the presently claimed and disclosed invention(s), a need existed in the art for a method of producing substantial quantities of individual HLA class I or class II molecules so that they can be readily purified and isolated independent of other HLA class I or class II molecules. Such individual isolated and purified HLA molecules, when provided in sufficient quantity and purity as described herein, provide a powerful tool for studying and measuring immune responses.

Therefore, there exists a need in the art for improved methods of isolating and purifying individual HLA molecules substantially away from other proteins. In one exemplary embodiment, the present invention solves this need by coupling the production of soluble HLA molecules with a purification methodology involving affinity chromatography.

### SUMMARY OF THE INVENTION

The present invention is directed to a functionally active, individual soluble HLA molecule purified substantially away from other proteins such that the individual soluble HLA molecule maintains the physical, functional and antigenic integrity of the native HLA molecule. The term “physical, functional and antigenic integrity of the native HLA molecule”, as used herein, will be understood to mean that the soluble HLA molecules exhibit the same structure (including primary, secondary, tertiary and quaternary) as the extracellular portion of the native HLA molecules, that they are identical in functional properties to an HLA molecule expressed from the HLA allele mRNA or gDNA and thereby bind peptide ligands in an identical manner as full-length, cell-surface-expressed HLA molecules, and that they are recognized by the cellular machinery responsible for responses to specific HLA-peptide complexes, that is NK and T cells.

The functionally active, individual soluble HLA molecule is a Class I HLA molecule or a Class II HLA molecule, and may have an endogenous peptide loaded therein.

The peptide may be produced by several methods, including but not limited to the following. In one embodiment, HLA allele mRNA from a source is isolated and reverse transcribed to obtain allelic cDNA. In a separate embodiment, gDNA encoding a HLA allele is obtained. The allelic cDNA or gDNA is amplified by PCR utilizing at least one locus-specific primer that truncates the allelic cDNA or gDNA, thereby resulting in a truncated PCR product having the coding regions encoding cytoplasmic and transmembrane domains of the allelic cDNA removed such that the truncated PCR product has a coding region encoding a soluble HLA molecule. The at least one locus-specific primer may include a stop codon incorporated into a 3′ primer, or the at least one locus-specific primer may include a sequence encoding a tail such that the soluble HLA molecule encoded by the truncated PCR product contains a tail attached thereto that facilitates in purification of the soluble HLA molecules produced therefrom.

The truncated PCR product is then inserted into a mammalian expression vector to form a plasmid containing the truncated PCR product having the coding region encoding a soluble HLA molecule, and the plasmid is electroporated into at least one suitable host cell. The mammalian expression vector contains a promoter that facilitates increased expression of the truncated PCR product. The host cell may lack expression of Class I HLA molecules.

A cell pharm is inoculated with the at least one suitable host cell containing the plasmid containing the truncated PCR product such that the cell pharm produces soluble HLA molecules, wherein the soluble HLA molecules are folded naturally and are trafficked through the cell in such a way that they are identical in functional properties to an HLA molecule expressed from the HLA allele mRNA and thereby bind peptide ligands in an identical manner as full-length, cell-surface-expressed HLA molecules. The individual, soluble HLA molecules are then harvested from the cell pharm and purified substantially away from other proteins. The purification process involves affinity column purification and filtration. The purified individual soluble HLA molecules maintain the physical, functional and antigenic integrity of the native HLA molecule.

When HLA allele mRNA is used, the source is selected from the group consisting of mammalian DNA and an immortalized cell line. When gDNA which encodes an HLA allele is used, the gDNA is obtained from blood, saliva, hair, semen, or sweat.

### BRIEF DESCRIPTION OF THE DRAWINGS

FIG. 1 is a graphical representation of a Class I location and sHLA class I construction strategy. (A) Simple map of the human MHC region with the class I HLA-B, -C, and -A loci noted. Genetic distances are in kilobases. (B) The basic exon structure of HLA class I gene transcripts. Seven exons encode the class I heavy chain. (C) PCR strategy for truncating the class I molecule so that it is secreted rather than surface bound.

FIG. 2 is a pictorial representation of native and recombined truncated form of sHLA which differ in the presence of a transmembrane and cytosolic region in the native molecule. Both forms show no differences in their ambiguity and peptide presenting properties.

FIG. 3 is a three dimensional pictorial representation of a truncated molecule. The bp view is visualizing the α₁ and α₂ domains harboring the peptide. The side view shows the full molecule with a detailed view of α₃ and β2m domains.

FIG. 4 is a pictorial representation showing the peptide binding platform in more detail where two α helices form the rim and seven β sheets form the bottom of the binding groove.

FIG. 5 is a graphical representation of an ELISA procedure demonstrating that W6/32-coupled affinity column can be saturated with crude harvest containing sHLA-B*0702His.

FIG. 6 is a graphical representation of an ELISA procedure demonstrating the wash step for the W6/32-coupled affinity column of FIG. 5.

FIG. 7 is a graphical representation of an ELISA procedure demonstrating the elution of sHLA-B*0702His from the W6/32-coupled affinity column of FIG. 5.

FIG. 8 is a chart showing the buffer exchange and concentration procedure using MACROSEP™ filters. ELISA performed during the filtration steps confirm minimal loss of protein.

FIG. 9 is a chart showing the final sterile filtration step optimized to remove remaining particles within the filtrate.

FIG. 10 is a tabular representation showing a summary of values measured during the purification procedure directly related to the efficiency.

FIG. 11 is a pictorial representation illustrating the Protein Sequence Data for MHC Class I-HLA-A*0201T.

FIG. 12 is a pictorial representation showing the Protein Sequence Data for MHC Class I-HLA-B*0702T.

FIG. 13 is a pictorial representation illustrating the Protein Sequence Data for MHC Class I-HLA-B*1512T.

FIG. 14 is a tabular representation illustrating the amino acid analysis of B*1512 following proteolysis of whole molecule.

FIG. 15 is a graphical representation showing Superdex™ chromatography to demonstrate sample purity of sHLA-B*1512T.

FIG. 16 is a graphical representation illustrating a Triple analysis of B*1512T. It shows a separation of sHLA under denaturing and under native conditions.

FIG. 17 is a graphical representation showing a Superdex™ profile of A*0201T.

FIG. 18 is a pictorial representation of an SDS-PAGE gel analysis of several purified sHLA samples confirming the purity with this procedure.

FIG. 19 is a pictorial representation of a Western blot analysis to follow the HC and β2m subunits of sHLA.

FIG. 20 is a chart depicting an activity confirmation of sHLA using standard sandwich ELISA procedure.

FIG. 21 is a pictorial scheme of antibody binding scenarios for the direct ELISA procedure. Several antibodies were tested on intact as well as denatured sHLA. Direct finding of sHLA molecules causes partial denaturization of the molecules and thus no specific denaturation step is necessary.

FIGS. 22-27 are charts showing reaction panels for conformation-specific Ab binding assays using the direct ELISA procedure.

FIG. 28 is a pictorial scheme of the two antibody binding scenarios using W6/32 or anti-b2m as capturing antibodies in a sandwich ELISA procure. Several detection antibodies were used.

FIGS. 29-32 are charts showing reaction panels for conformation-specific Ab binding assays using several Pan-Class I monoclonal antibodies in the sandwich ELISA procedure.

FIGS. 33-34 are charts illustrating various antibody combinations to test for artificial structural forms such as aggregation or dimeric structures showing A, B, and C alleles.

FIGS. 35-36 are charts illustrating neutralization experiments to verify antigenic integrity using sHLA-A*0201T and A2 alloantiserum M102 as well as Ab MA2.1.

FIG. 37 is a pictorial representation illustrating anti-calreticulin blot of full-length. HLA-B27 (+), HLA negative cell line 721.221 (−) and various constructs of soluble HLA-B15 molecules immunoprecipitated with the HLA-specific antibody HC-10.

FIGS. 38-51 are charts showing ELISA reactions testing a panel of selected sHLA alleles using different commercially available single specificity monoclonal antibodies.

FIGS. 52-53 are charts illustrating ELISA Reaction panels testing antibodies Bw6 and Bw4.

FIG. 54 is a pictorial representation depicting a motif comparison between sHLA-B*1501 and membrane bound B*1501 from another laboratory.

FIG. 55 is a pictorial representation showing a fluorescence polarization scheme allowing the detection of bound and free peptides to the sHLA complex in solution without separation using radiometric measurements of parallel and perpendicular fluorescent intensities. Free peptides create a low FP signal where bound peptides show high FP values.

FIGS. 56-57 are graphical representations showing a one phase exponential association curve using the sHLA allele A*0201T combined with the FITC-labeled peptide P5 (A*0201).

FIGS. 58-59 are graphical representations showing saturation experiments generating saturation curve wherein sHLA (binder) is held constant to determine the dissociation constant (K_{D}).

FIGS. 60-61 are graphical representations showing competition experiments of fixed concentration of fluorescent-labeled synthetic peptide in the presence of various concentrations of unlabeled test competitor-peptides to determine the IC₅₀ value.

FIG. 62 is a graphical representation showing an ELISA procedure demonstrating the binding of a HBV peptide to sHLA molecules and successful replacement of the endogenous peptide with the HBV peptide.

FIGS. 63-66 are charts showing ELISA procedures demonstrating stability of sHLA-B*1512T in different buffers and solutions during different days with a summary given in FIG. 66.

FIG. 67 is a graphical representation showing an ELISA procedure demonstrating the influence of temperature on stability of sHLA complex.

FIG. 68 is a graphical representation showing the influence of freeze-thaw cycle on stability.

FIG. 69 is a pictorial representation showing the experimental procedure for determining loss of complex reactivity due to nonspecific adhesion to surfaces of tubes.

FIG. 70 is a chart showing the effects of different microcentrifuge tubes or cryo vials on reactivity of sHLA.

FIG. 71 is a chart showing the effects of larger tubes on reactivity of sHLA.

FIGS. 72-73 are charts depicting the effects of blocking agents on reactivity of sHLA, including PVP and PEG.

FIG. 74 is a chart showing the effects of non-ionic detergents on reactivity of sHLA.

FIG. 75 is a chart showing the effect of different BSA concentrations on reactivity of sHLA.

FIG. 76 is a chart showing the effect of different Stabilguard™ concentrations on reactivity of sHLA.

FIG. 77 is a chart showing the effect of PEG concentrations on reactivity of sHLA.

FIG. 78 is a chart showing the effect of PVP concentrations on reactivity of sHLA.

FIGS. 79-85 are charts illustrating a sera screen assay that utilizes HLA to identify antigen-specific antibodies in human sera.

FIG. 86 is a chart showing SHLA A*0201T reactivity on beads sampled through the EDC method.

FIG. 87 is a graphical representation depicting the screening of test competitors for ability to inhibit FITC-labeled standard peptide from binding to sHLA.

FIG. 88 is a graphical representation showing constructed IC₅₀ binding curves using a single inhibition value obtained at 100 μM competitor concentration.

FIG. 89 is a graphical representation showing IC₅₀ values obtained during the single value procedure as well as the more accurate 9 point procedure sorted according to their measured affinities.

FIGS. 90-91 are graphical representations illustrating the improvement of binding of modified peptides to sHLA-A2 as compared to the native test-peptides Vac 104 and Vac 105.

FIG. 92 is a graphical representation summarizing the purification and characterization procedures for soluble human HLA proteins of the present invention.

### DETAILED DESCRIPTION OF THE INVENTION

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

The present invention combines methodologies for the production of individual, soluble MHC molecules with novel and nonobvious methodologies for the isolation and purification of individual, soluble MHC molecules substantially away from other proteins. The method of production of individual, soluble MHC molecules has previously been described in detail in parent application U.S. Ser. No. 10/022,066, filed Dec. 18, 2001, entitled “METHOD AND APPARATUS FOR THE PRODUCTION OF SOLUBLE MHC ANTIGENS AND USES THEREOF,” the contents of which are hereby expressly incorporated in their entirety by reference herein. A brief description of this methodology is included herein below for the purpose of exemplification and should not be considered as limiting. One of ordinary skill in the art, given the disclosure in the U.S. Ser. No. 10/022,066 application would be truly capable of producing individual soluble MHC molecules to be used with the presently disclosed and claimed isolation and purification methodologies. It should be preliminary noted, however, that the presently claimed and disclosed isolation and purification methodologies can be used with HLA molecules (soluble or non-soluble) obtained by any means and should not be regarded as being limited to soluble HLA molecules produced according to the methodologies claimed and disclosed in the U.S. Ser. No. 10/022,066 application. In the event HLA molecules produced according to methodologies other than those produced according to methodologies disclosed and claimed in the U.S. Ser. No. 10/022,066 application are used in the isolation and purification methodologies disclosed and claimed herein, one of ordinary skill in the art (given in the present specification, drawings and claims) would be capable of making any necessary modifications or derivations to such HLA molecules such that they may be used in the isolation and purification methodologies presently claimed and disclosed herein in an efficient and accurate manner.

Exemplary Production of Individual, Soluble MHC Molecules

The methods of the present invention may, in one embodiment, utilize a method of producing MHC molecules (from genomic DNA or cDNA) that are secreted from mammalian cells in a bioreactor unit. Substantial quantities of individual MHC molecules may be obtained in the manner by more particularly modifying class I or class II MHC molecules so that they are capable of being secreted, isolated, and purified. Secretion of soluble MHC molecules overcomes the disadvantages and defects of the prior art in relation to the quantity and purity of MHC molecules produced. Problems of quantity are overcome because the cells producing the MHC do not need to be detergent lysed or killed in order to obtain the MHC molecule. In this manner, the cells producing secreted MHC remain alive and therefore continue to produce MHC. Problems of purity are overcome because the only MHC molecule secreted from the cell is the one that has specifically been constructed to be secreted. Thus, transfection of vectors encoding such secreted MHC molecules into cells which may express endogenous, surface bound MHC provides a method of obtaining a highly concentrated form of the transfected MHC molecule as it is secreted from the cells. Greater purity is assured by transfecting the secreted MHC molecule into MHC deficient cell lines.

Production of the MHC molecules in a hollow fiber bioreactor unit allows cells to be cultured at a density substantially greater than conventional liquid phase tissue culture permits. Dense culturing of cells secreting MHC molecules further amplifies the ability to continuously harvest the transfected MHC molecules. Dense bioreactor cultures of MHC secreting cell lines allow for high concentrations of individual MHC proteins to be obtained. Highly concentrated individual MHC proteins provide an advantage in that most downstream protein purification strategies perform better as the concentration of the protein to be purified increases. Thus, the culturing of MHC secreting cells in bioreactors allows for a continuous production of individual MHC proteins in a concentrated form.

While hollow fiber bioreactor units or cell pharms have been described herein for utilization in the culturing methods of the present invention, it is to be understood that any large scale mammalian tissue culture system evident to a person having ordinary skill in the art may be utilized in the methods of the present invention, and therefore the present invention is not specifically limited to the use of a hollow fiber bioreactor unit or a cell pharm.

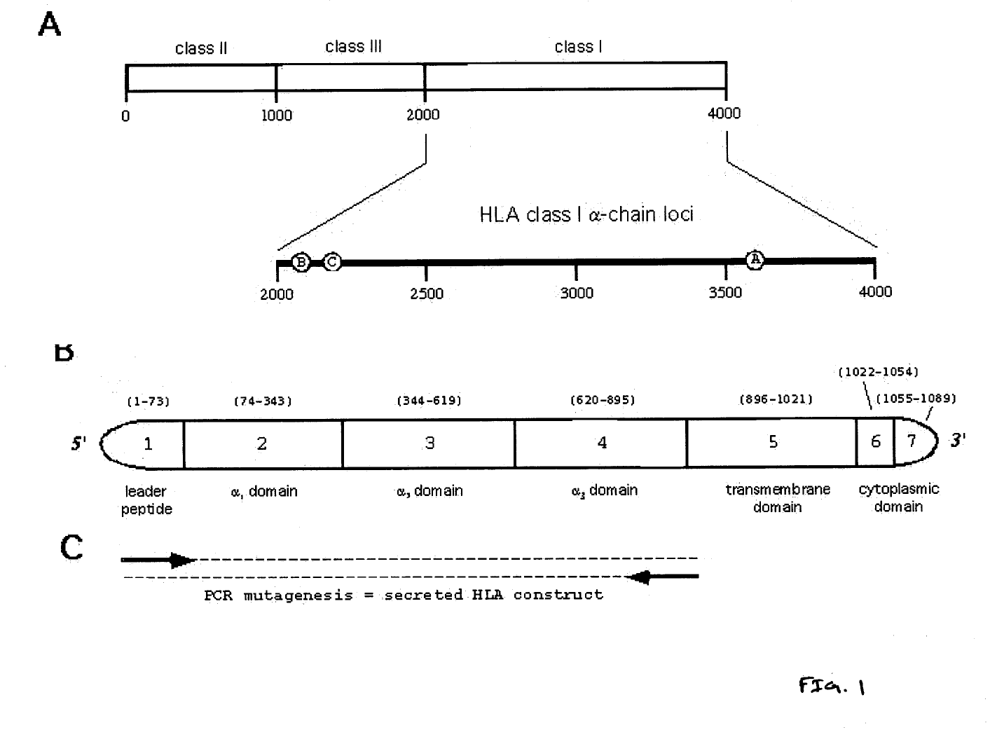

The method of producing MHC molecules utilized in the present invention and described in detail in parent application U.S. Ser. No. 10/022,066 begins by obtaining genomic or complementary DNA which encodes the desired MHC class I or class II molecule. Alleles at the locus which encode the desired MHC molecule are PCR amplified in a locus specific manner. These locus specific PCR products may include the entire coding region of the MHC molecule or a portion thereof. In one embodiment a nested or hemi-nested PCR is applied to produce a truncated form of the class I or class II gene so that it will be secreted rather than anchored to the cell surface. FIG. 1 illustrates the PCR products resulting from such nested PCR reactions. In another embodiment the PCR will directly truncate the MHC molecule.

Locus specific PCR products are cloned into a mammalian expression vector and screened with a variety of methods to identify a clone encoding the desired MHC molecule. The cloned MHC molecules are DNA sequenced to ensure fidelity of the PCR. Faithful truncated clones of the desired MHC molecule are then transfected into a mammalian cell line. When such cell line is transfected with a vector encoding a recombinant class I molecule, such cell line may either lack endogenous class I MHC molecule expression or express endogenous class I MHC molecules.

One of ordinary skill in the art would note the importance, given the present invention, that cells expressing endogenous class I MHC molecules may spontaneously release MHC into solution upon natural cell death. In cases where this small amount of spontaneously released MHC is a concern, the transfected class I MHC molecule can be “tagged” such that it can be specifically purified away from spontaneously released endogenous class I molecules in cells that express class I molecules. For example, a DNA fragment encoding a HIS tail may be attached to the protein by the PCR reaction or may be encoded by the vector into which the PCR fragment is cloned, and such HIS tail, therefore, further aids in the purification of the class I MHC molecules away from endogenous class I molecules. Tags beside a histidine tail have also been demonstrated to work, and one of ordinary skill in the art of tagging proteins for downstream purification would appreciate and know how to tag a MHC molecule in such a manner so as to increase the ease by which the MHC molecule may be purified.

Cloned genomic DNA fragments contain both exons and introns as well as other non-translated regions at the 5′ and 3′ termini of the gene. Following transfection into a cell line which transcribes the genomic DNA (gDNA) into RNA, cloned genomic DNA results in a protein product thereby removing introns and splicing the RNA to form messenger RNA (mRNA), which is then translated into an MHC protein. Transfection of MHC molecules encoded by gDNA therefore facilitates reisolation of the gDNA, mRNA/cDNA, and protein. Production of MHC molecules in non-mammalian cell lines such as insect and bacterial cells requires cDNA clones, as these lower cell types do not have the ability to splice introns out of RNA transcribed from a gDNA clone. In these instances the mammalian gDNA transfectants of the present invention provide a valuable source of RNA which can be reverse transcribed to form MHC cDNA. The cDNA can then be cloned, transferred into cells, and then translated into protein. In addition to producing secreted MHC, such gDNA transfectants therefore provide a ready source of mRNA, and therefore cDNA clones, which can then be transfected into non-mammalian cells for production of MHC. Thus, the present invention which starts with MHC genomic DNA clones allows for the production of MHC in cells from various species.

A key advantage of starting from gDNA is that viable cells containing the MHC molecule of interest are not needed. Since all individuals in the population have a different MHC repertoire, one would need to search more than 500,000 individuals to find someone with the same MHC complement as a desired individual—such a practical example of this principle is observed when trying to find a donor to match a recipient for bone marrow transplantation. Thus, if it is desired to produce a particular MHC molecule for use in an experiment or diagnostic, a person or cell expressing the MHC allele of interest would first need to be identified. Alternatively, in the method of the present invention, only a saliva sample, a hair root, an old freezer sample, or less than a milliliter (0.2 ml) of blood would be required to isolate the gDNA. Then, starting from gDNA, the MHC molecule of interest could be obtained via a gDNA clone as described herein, and following transfection of such clone into mammalian cells, the desired protein could be produced directly in mammalian cells or from cDNA in several species of cells using the methods described herein.

Current experiments to obtain an MHC allele for protein expression typically start from mRNA, which requires a fresh sample of mammalian cells that express the MHC molecule of interest. Working from gDNA does not require gene expression or a fresh biological sample. It is also important to note that RNA is inherently unstable and is not as easily obtained as is gDNA. Therefore, if production of a particular MHC molecule starting from a cDNA clone is desired, a person or cell line that is expressing the allele of interest must traditionally first be identified in order to obtain RNA. Then a fresh sample of blood or cells must be obtained; experiments using the methodology of the present invention show that ≧5 milliliters of blood that is less than 3 days old is required to obtain sufficient RNA for MHC cDNA synthesis. Thus, by starting with gDNA, the breadth of MHC molecules that can be readily produced is expanded. This is a key factor in a system as polymorphic as the MHC system; hundreds of MHC molecules exist, and not all MHC molecules are readily available. This is especially true of MHC molecules unique to isolated populations or of MHC molecules unique to ethnic minorities. Starting class I or class II MHC molecule expression from the point of genomic DNA simplifies the isolation of the gene of interest and insures a more equitable means of producing MHC molecules for study; otherwise, one would be left to determine whose MHC molecules are chosen and not chosen for study, as well as to determine which ethnic population from which fresh samples cannot be obtained and therefore should not have their MHC molecules included in a diagnostic assay.

While cDNA may be substituted for genomic DNA as the starting material, production of cDNA for each of the desired HLA class I types will require hundreds of different, HLA typed, viable cell lines, each expressing a different HLA class I type. Alternatively, fresh samples are required from individuals with the various desired MHC types. The use of genomic DNA as the starting material allows for the production of clones for many HLA molecules from a single genomic DNA sequence, as the amplification process can be manipulated to mimic recombinatorial and gene conversion events. Several mutagenesis strategies exist whereby a given class I gDNA clone could be modified at either the level of gDNA or at the cDNA resulting from this gDNA clone. The process of producing MHC molecules utilized in the present invention does not require viable cells, and therefore the degradation which plagues RNA is not a problem.

Purification of Individual, Soluble MHC Molecules

The ability to produce large quantities of single specificity sHLA molecules allows for assay procedures to be quantitative and resistant to interferences encountered in biological matrices as well as also being reliable, highly reproducible, sensitive, and therefore applicable for high-throughput systems. Alternative economical methodologies for obtaining large quantities of sHLA molecules do not currently exist since: (1) there is no readily available source of individual HLA molecules; (2) purification of native class I molecules from mammalian cells requires time-consuming and cumbersome purification methods and does not deliver sufficient quantities; and (3) native molecules from mammalian cells typically consist of a mixture of different HLA molecules. Such a mixture of specificities is not useful and/or applicable for single specificity studies.

HLA class I molecules are antigen-presenting glycoproteins expressed universally in nucleated cells. In humans, heavy chains are encoded at 3 loci (B, C, and A) within the MHC on the short arm of chromosome 6 (FIG. 1A). FIG. 1B illustrates each a-chain comprised of α₁, α₂, and α₃ domains, as well as a transmembrane domain, which tethers the molecule to the cell surface and a short C-terminal cytoplasmic domain. In contrast, the light chain is encoded outside of the MHC (on chromosome 15 in humans) and bears no such anchoring domain; it instead associates noncovalently with the α₃ domain of the heavy chain. FIG. 1C illustrates the approach for creating sHLA class I transcripts. The PCR primers truncate the class I heavy chain following exon 4, just before the transmembrane domain and cytoplasmic domains. Using this PCR truncation strategy, we have successfully created sHLA class I gene products for a series of fifty divergent HLA-molecules. Class I sHLA gene constructs created as in FIG. 1C are cloned and DNA sequenced to insure fidelity of each clone. The individual class I constructs are then subcloned into a suitable protein expression vector.

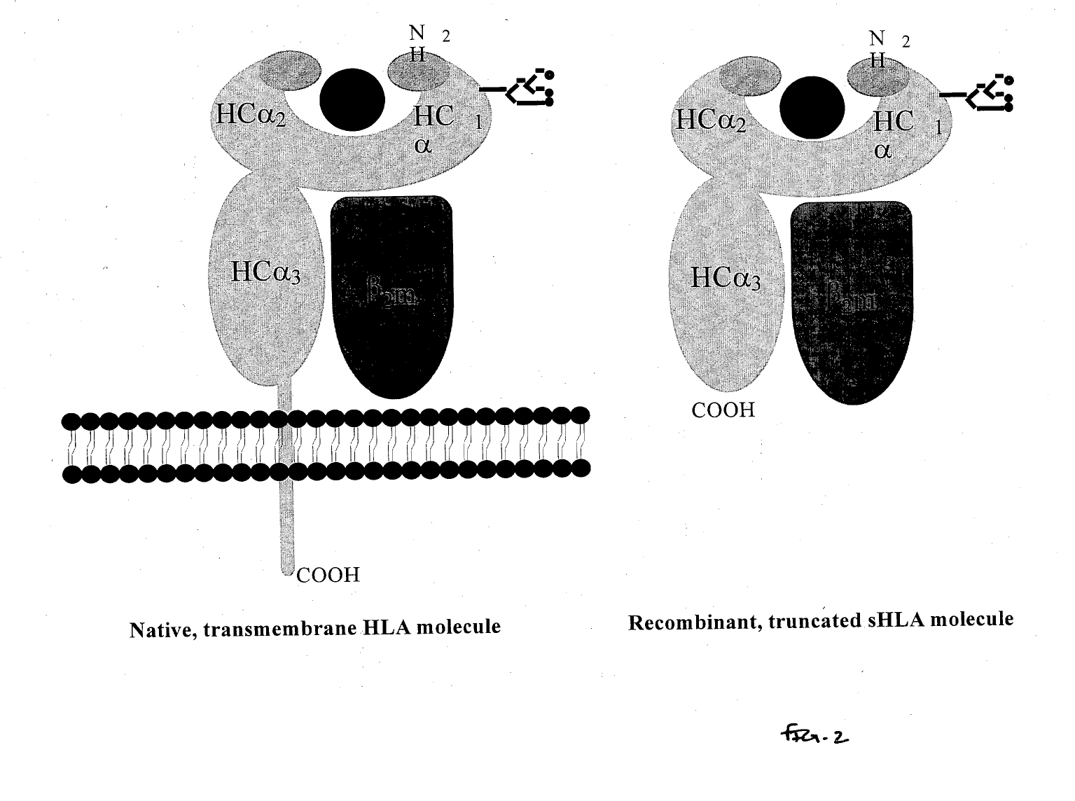

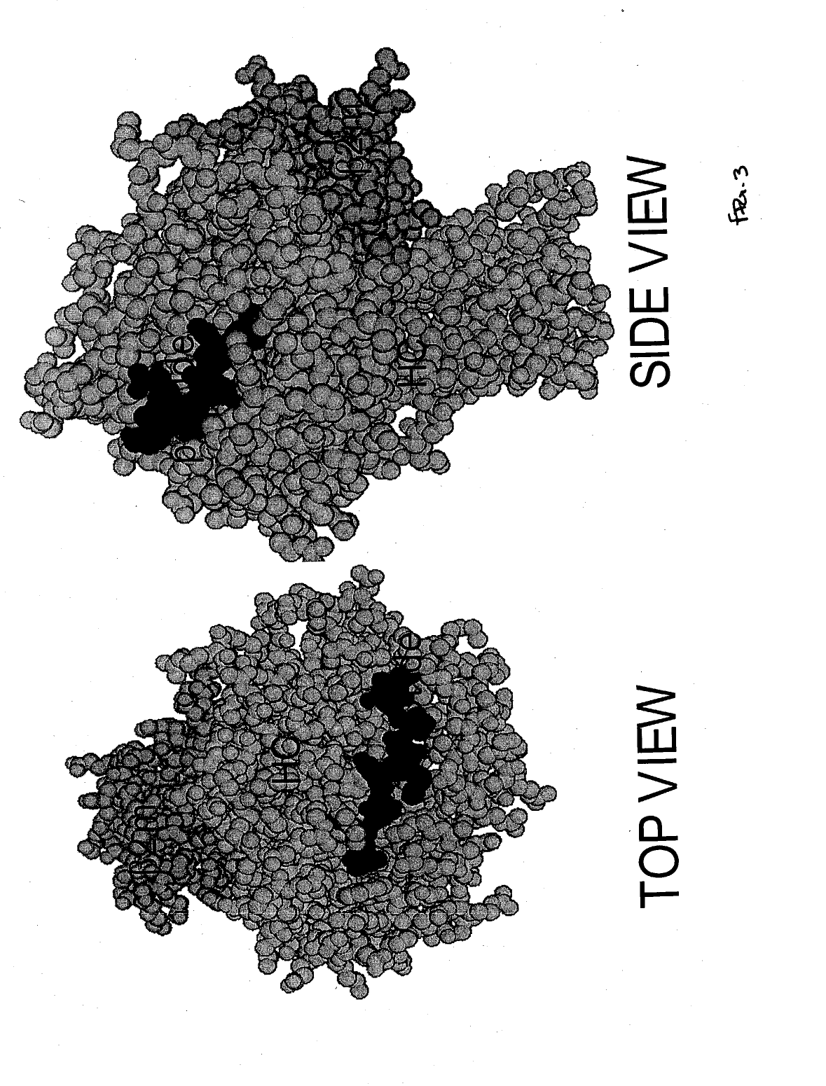

Produced in transfected B cells, sHLA molecules have close to identical primary structures as papain solubilized HLAs. Truncated molecules have been shown by the present inventors to maintain their structural integrity. In addition, HLA-Aw68, from which the complete alpha 3 domain has been proteolytically removed, shows no gross morphological changes compared to the intact protein. A decameric peptide complexed with the intact HLA-Aw68 is seen to bind to the proteolized molecule in the conventional manner, demonstrating that the alpha 3 domain is not required for the structural integrity of the molecule or for peptide binding. Pictures of sHLA graphics (FIG. 2) and 3D structures (FIG. 3) more clearly visualize how the molecules look like.

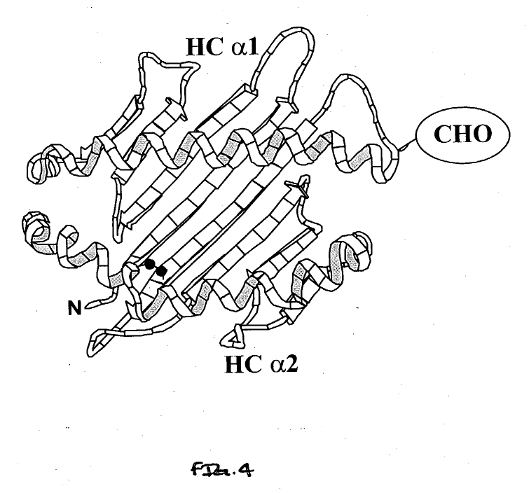

HLA/MHC genes are the most polymorphic system in mammals, generated by systematic recombinatorial and point mutation events; as such, hundreds of different HLA types exist throughout the world's population, resulting in a large immunological diversity. Individuals inherit a set of three class I genes from each parent, and since their expression is codominant, a single person may therefore display up to six different HLA class I molecules upon his or her nucleated cells. Such extensive HLA diversity results in differing susceptibilities and/or resistances between individuals in infectious diseases. Depending upon allelic composition, two individuals' molecules may not necessarily bind the same peptides with equal affinity or even at all. Therefore, despite the overall structural conservation illustrated among class I heavy chains, their peptide binding grooves can vary drastically from one allelic form to another; as a result various isoforms are capable of associating with distinct arrays of peptides. A binding platform is shown in FIG. 4. The first two domains (alpha 1, alpha 2) of the heavy chain create the peptide binding cleft and the surface that contacts the T-cell receptor. X-ray crystallographic analysis indicates that a processed antigen is presented as a peptide bound in a cleft between the two α-helices of the heavy chain of the HLA complex (Bjorkman P. J., 1987; Nature 329: 506-512 & 512-518/Garett T. J. 1989: Nature 342; 692-696/Saper M. A.; 1991; J. Mol. Biol. 219; 277-319/Madden D. R. (1991) Nature 353; 321-325; the contents of each are herein expressly incorporated by reference in their entirety.). The third domain (alpha 3) associates with the T-cell co-receptor, CD8, during T-cell recognition. Availability of a wide spectrum of recombinant sHLA molecules overcomes the current art limitations on population coverage imposed by the rules of MHC restriction. In most cases, a single-peptide epitope will be useful only for treating a small subset of patients who express the MHC allele product that is capable of binding that specific peptide. Since every individual has differing MHC molecules, the testing of numerous individual MHC molecules is a prerequisite for understanding the difference in disease susceptibility between individuals.

Purification Methodology

There are many purification methods available for the separation of macromolecules. To effectively resolve a crude mixture of substances, it may be necessary to use a combination of techniques. In most cases, a purification procedure will involve some chromatographic techniques.

Affinity chromatography occupies a unique place in separation technology since it is the only technique which enables purification of almost any biomolecule on the basis of its biological function or individual chemical structure. Affinity chromatography makes use of specific binding interactions that occur between molecules. It is a type of adsorption chromatography in which the molecule to be purified is specifically and reversibly adsorbed by a complementary binding substance (ligand) immobilized on an insoluble support (matrix). A single pass through an affinity column can achieve a 1,000-10,000 fold purification of ligand from a crude mixture. It is possible to isolate a compound in a form pure enough to obtain a single band upon SDS-polyacrylamide gel electrophoresis. Any component that has an interacting counterpart can be attached to a support and used for affinity purification.

Successful separation by affinity chromatography requires that a biospecific ligand is available and that it can be covalently attached to a chromatographic bed material called a matrix. It is important that the biospecific ligand (antibody, enzyme, or receptor protein) retains its specific binding affinity for the substance of interest (antigen, substrate, or hormone). Methods must also include removing the bound material in active form with low pH, high pH, or high salt. The selection of the ligand for affinity chromatography is influenced by two factors. Firstly, the ligand should exhibit specific and reversible binding affinity for the substance to be purified. Secondly, it should have chemically modifiable groups, which allow it to be attached to the matrix without destroying its binding activity. The ligand should ideally have an affinity for the binding substance in the range 10⁻⁴ to 10⁻⁸ M in free solution.

The protocol herein discussed provides a method to couple protein to a commercially available CNBr-activated Sepharose 4B (APB #17-0430-01). An alternative option would be running the procedure with Sepharose 4 Fast flow (APB #17-0981-01). Sepharose Fast Flow is more highly crosslinked than Sepharose 4B. As a result, Fast Flow beads are more stable and can withstand higher flow rates than the 4B beads. CNBr-activated Sepharose 4B is better suited for batch chromatography and small columns with gravity flow. Another difference is in coupling capacities. The coupling reaction proceeds most efficiently in the pH range 8-10 where the amino groups on the ligand are predominantly in the unprotonated form. A buffer at pH 8.3 is most frequently used for coupling proteins. IgGs are often coupled at a slightly higher pH, for example in a NaHCO₃ buffer (0.2-0.25 M) containing 0.5 M NaCl, at pH 8.5-9.0. Carbonate/bicarbonate and borate buffer systems with the addition of NaCl may be used. The coupling buffer solution should have a high salt content (about 0.5 M NaCl) to minimize protein-protein adsorption caused by the polyelectrolyte nature of proteins. Coupling at low pH is less efficient but may be advantageous if the ligand loses biological activity when it is fixed too firmly, e.g. by multi-point attachment, or because of steric hindrance between binding sites which occurs when a large amount of high molecular weight ligand is immobilized. A buffer of approximately pH 6 is used. Tris and other buffers containing amino groups must not be used at this stage since these buffers will couple to the gel.

Protein coupled to CNBr-activated Sepharose™ 4B is usually more stable to denaturation than the protein in free solution, but reasonable care in the choice of storage conditions should be exercised. Suspensions should be stored in a refrigerator below 4° C. in the presence of a suitable bacteriostatic agent. The choice of buffer solution depends on the properties of the particular coupled protein.

In affinity chromatography, nonspecific proteins flow through the column while the specific protein is retained by the column. The protein is then eluted, and individual fractions are tested for specific-binding activity and purity. Several different approaches can be taken to allow efficient binding of antigens to immunoaffinity columns. Because the antibody is not in solution, the time required for the antibody-matrix/antigen interaction will have different kinetics than soluble interactions. It will take considerably longer for equilibrium to be reached than for solution assays. Therefore, the binding protocol should maximize the degree of interaction. The recommended method is binding by passing the antigen solution down an antibody-matrix column, keeping the antigen in contact with the antibody for as long as possible. In this case, high-affinity antibodies will be significantly more efficient at removing the antigen from solution than low-affinity antibodies. Several small-scale columns can be used to determine the best conditions for binding and collecting the antigen.

Although the exact affinity of an antibody for an antigen can be calculated, for most work the crucial criterion is whether the antibodies will remove the antigen from solution quantitatively. The easiest method to test this is to set up small-scale reactions and examine the first wash buffer for the presence of the antigen. The amount of bound antigen may be increased by using higher amounts of antibodies on the beads, by increasing the number of beads, or by increasing the amount of time for binding. Unfortunately, all of these conditions will raise the nonspecific background, so a compromise normally will result in the highest yields with the lowest acceptable background. Use of high-affinity antibodies solves the problem of efficiently collecting the antigen. Consequently, they can be used in dilute solutions, at relatively lower antigen. Consequently, they can be used in dilute solutions, at relatively lower concentrations, and for shorter times.

A titration can be performed as a first step in estimating the ratio of column matrix needed to bind a given amount of antigen. This can be handled where an equal volume of the antibody/Sepharose 4B matrix is added to samples containing increasing concentrations of the antigen. The slurry is mixed at 4° C. for 1 hr and then processed. This will yield a rough idea of the volume of column matrix needed to collect the desired amount of antigen. If the supernatants from the binding reaction are assayed for the presence of the antigen, the extent of antigen depletion also can be determined.

Developing the best elution conditions is an empirical task determined by testing a series of buffers. Three types of elution are possible. The antigen-antibody interactions can be broken by (1) treating with harsh conditions, (2) adding a saturating amount of a small compound that mimics the binding site, and/or (3) treating with an agent that induces an allosteric change that releases the antigen. The most commonly used elution procedure relies on breaking the bonds between the antibody and antigen. The elutions may be harsh, denaturing the antibody and the antigen, or mild, leaving both the antigen and antibody in active states.

The mildest elution conditions are required if the protein of interest is labile. Avoid dithiothreitol and other reducing agents, as they will break disulfide linkages. Any buffers that fail to elute the antigen should be considered as good candidates for wash buffers. Some noneluting buffers may, in fact, drive the antibody-antigen equilibrium toward complex formation. The usual procedure when elution conditions have not been defined is to try the mildest elution conditions first and proceed to harsher treatments. If trying for the gentlest elution conditions, start with acid conditions first, then check basic elution buffers. If these conditions do not elute the antigen, try others. A general order to check the various conditions would be:

| | | |
|---|---|---|
| Low pH | acid, pH 3-1.5 | |
| | | 0.1 M glycine-HCl (pH 2.5) |
| | | 0.1 M glycine sulfate (pH 2.3) |
| | | 0.1 M propionic acid (pH 2.3) |
| | | 3.0 M KSCN (pH 2.3) |
| High pH | base, pH 10-12.5 | |
| | | 0.1 M glycine-NaOH (pH 11.0) |
| | | 0.15 M NH₄OH (pH 10.5) |
| Chaotropic Agents | MgCl₂, 3-5 M | 4 M MgCl₂ in 10 mM PBS (pH 7.0) |
| | LiCl 5-10 M | |
| | Water | |
| Polarity-reducing Agents | Ethylene glycol 25-50% | |
| | Dioxane 5-20% | |
| Denaturing Agents | Thiocyanate 1-5 M | |
| | Guanidine 2-5 M | |
| | Urea 2-8 M | |
| | SDS 0.5-2% | |

Microconcentrators are used primarily for removal of excess salts in protein purification or analysis. A variety of materials have been used to fabricate these semipermeable membranes, ranging from cellulose and cellulose esters to polyethersulfone (PES) or polyvinylidene difluoride (PVDF). All membranes are characterized by their molecular-weight cutoff (MWCO) value. This is usually defined as the molecular weight of a solute that is 90% prevented from penetrating the membrane under a chosen set of conditions. How readily a particular protein is rejected by the membrane is a function of the shape, hydration state, and charge of the protein molecule. Moreover, MWCO values are not sharp; rather, there is a gradual increase in retention as the size of solute molecules approaches and exceeds the average membrane pore size. Only at the point where all pores are smaller than a particular solute molecule is that molecule completely excluded.

The advantage of desalting processes based on ultrafiltration over those based on simple dialysis is that the rate of low-molecular-weight solute removal is not determined by a concentration differential, but rather by the flow rate of solvent and the rejection of the solute by the ultrafiltration membrane employed. Membranes for ultrafiltration are generally selected on the basis of the MWCO needed to retain the protein of interest but allow the maximum amount of other materials to pass through. It is usually best to choose an MWCO value that is roughly one-half the molecular weight of the species to be retained. This provides a reasonable margin of retention whereby almost none of the protein of interest should be lost, but at the same time provides the largest difference between the MWCO value and the molecular weight of the salts to be removed, thereby maximizing filtration rate.

In regard to the degree of nonspecific adsorption of protein to membranes, losses of 1% to 5% are not uncommon when dealing with total quantities of protein in the range of 1 to 10 mg using a filter with a 43-mm diameter. The nature of the buffer can also affect adsorption of protein; some membranes exhibit altered flow properties when high levels of ions are present. In this regard, phosphate buffers seem to present more of a problem than Tris buffers. The degree of concentration to be achieved by ultrafiltration should be that required for subsequent work. Recovery of sample following concentration is generally 95%; failure to achieve this value usually indicates leakage into the filtrate or nonspecific binding to the membrane and/or concentration apparatus itself.

At a constant temperature and pressure, the flow rate is a function of the filter area and the degree to which concentration polarization can be avoided. Buildup of protein on the surface will result in slow filtration, even when the protein concentration of the sample is relatively low. Filtration rates at 4° C. are often only one-half those seen at 25° C. because of the influence of viscosity. For biochemical analysis, monomorphic monoclonal antibodies are particularly useful for identification of HLA locus products and their subtypes.

W6/32 is one of the most common monoclonal antibodies (mAb) used to characterize human class I major histocompatibility complex (MHC) molecules. This antibody recognizes only mature complexed class I molecules. It is directed against a conformational epitope on the intact MHC molecule that includes both residue 3 of beta2m and residue 121 of the heavy chain (Ladasky J J, Shum B P, Canavez F, Seuanez H N, Parham P. Residue 3 of beta2-microglobulin affects binding of class I MHC molecules by the W6/32 antibody. Immunogenetics 1999 April; 49(4):312-20, the contents of which is expressly incorporated herein by reference in its entirety.). The constant portion of the molecule W6/32 binds to is recognized by CTLs and thus can inhibit cytotoxicity. The reactivity of W6/32 is sensitive to the amino terminus of human beta2-microglobulin (Shields M I, Ribaudo R K. Mapping of the monoclonal antibody W6/32: sensitivity to the amino terminus of beta2-microglobulin. Tissue Antigens 1998 May; 51(5):567-70, the contents of which is expressly incorporated herein by reference in its entirety.). HLA-C could not be clearly identified in immunoprecipitations with W6/32 suggesting that HLA-C locus products may be associated only weakly with β2m, explaining some of the difficulties encountered in biochemical studies of HLA-C antigens. The polypeptides correlating with the C-locus products are recognized far better by HC-10 than by W6/32 which seems to confirm that at least some of the C products may be associated with β2m more weakly than HLA-A and -B.

HC-10 is reactive with almost all HLA-B locus free heavy chains. The A2 heavy chains are only very weakly recognized by HC-10. Moreover, HC-10 reacts only with a few HLA-A locus heavy chains. In addition, HC-10 seems to react well with free heavy chains of HLA-C types. No evidence for reactivity of HC-10 with heavy-chain/β2m complex was obtained. None of the immunoprecipitates obtained with HC-10 contained β2m. This suggests that HC-10 is directed against a site of the HLA class I heavy chain that might include the portion involved in interaction with the β2m. The pattern of HC-10 precipitated material is qualitatively different from that isolated with W6/32.

TP25.99 detects a determinant in the alpha3 domain of HLA-ABC. It is found on denatured HLA-B (in Western) as well as partially or fully folded HLA-A, B, & C. It doesn't require a peptide or β2m, i.e. it works with the alpha 3 domain which folds without peptide. This makes it useful for HC determination.

Anti-human β2m (HRP) (DAKO P0174) recognizes denatured as well as complexed β2m. Although in principle anti-β2m reagents could be used for the purpose of identification of HLA molecules, they are less suitable when association of heavy chain and β2m is weak. The patterns of class I molecules precipitated with W6/32 and anti-β2m are usually indistinguishable.

### EXPERIMENTAL EXAMPLES OF THE PRESENT INVENTION

Purification of Individual, Soluble MHC Molecules

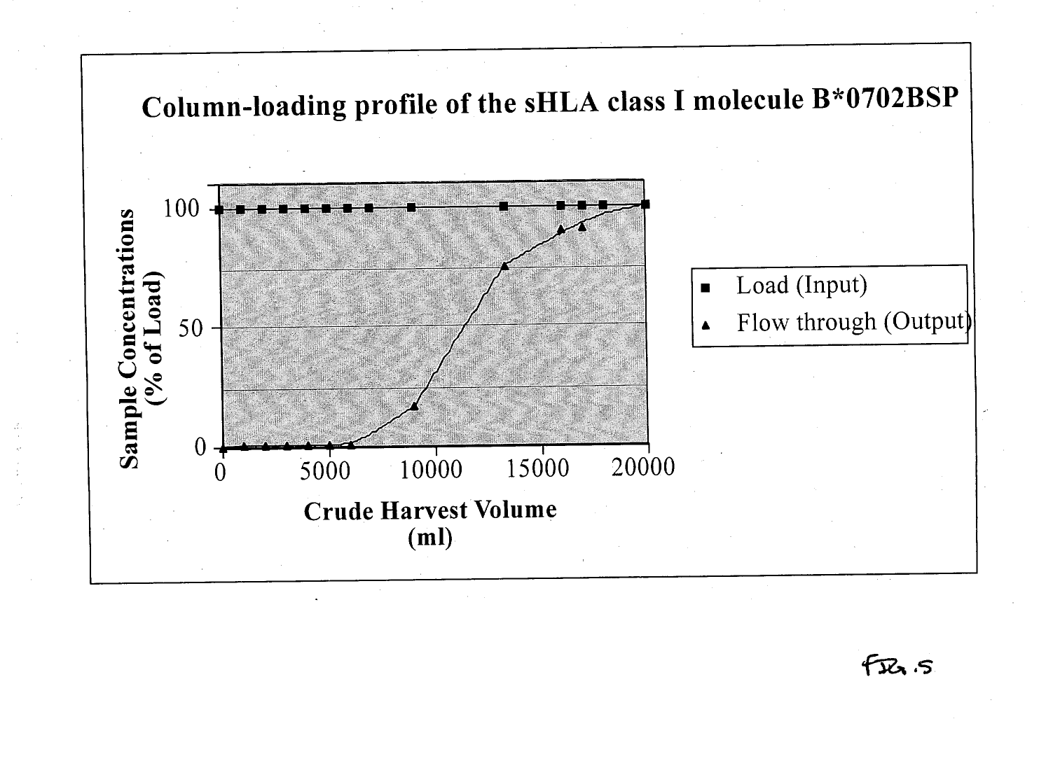

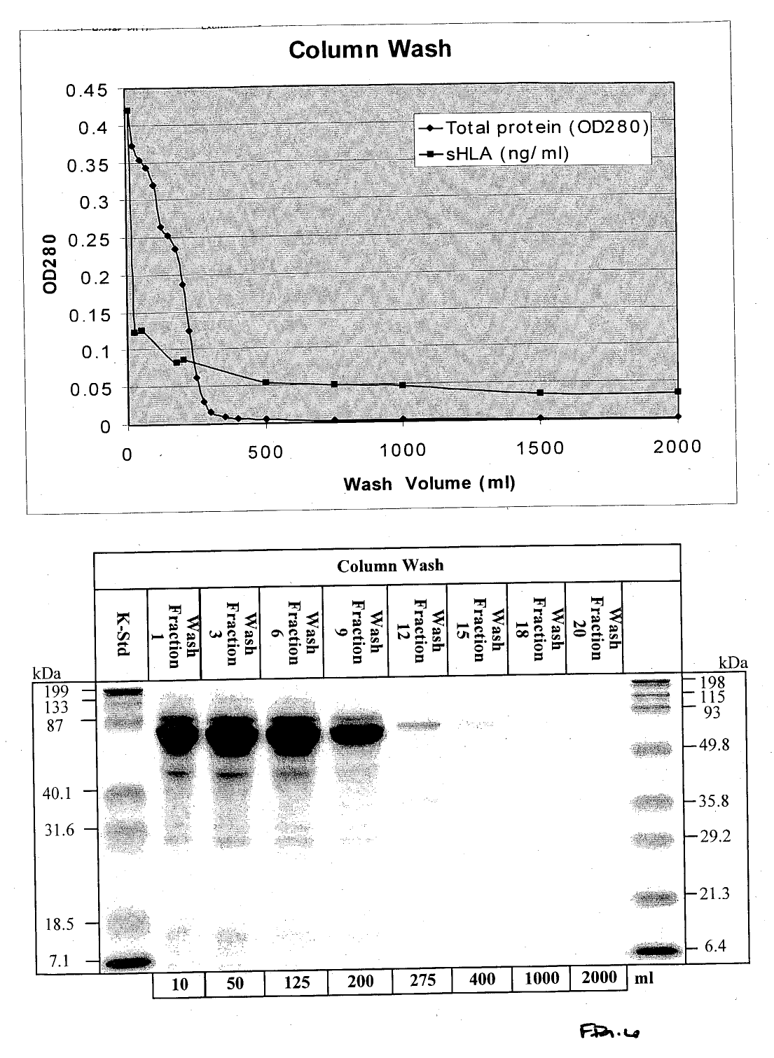

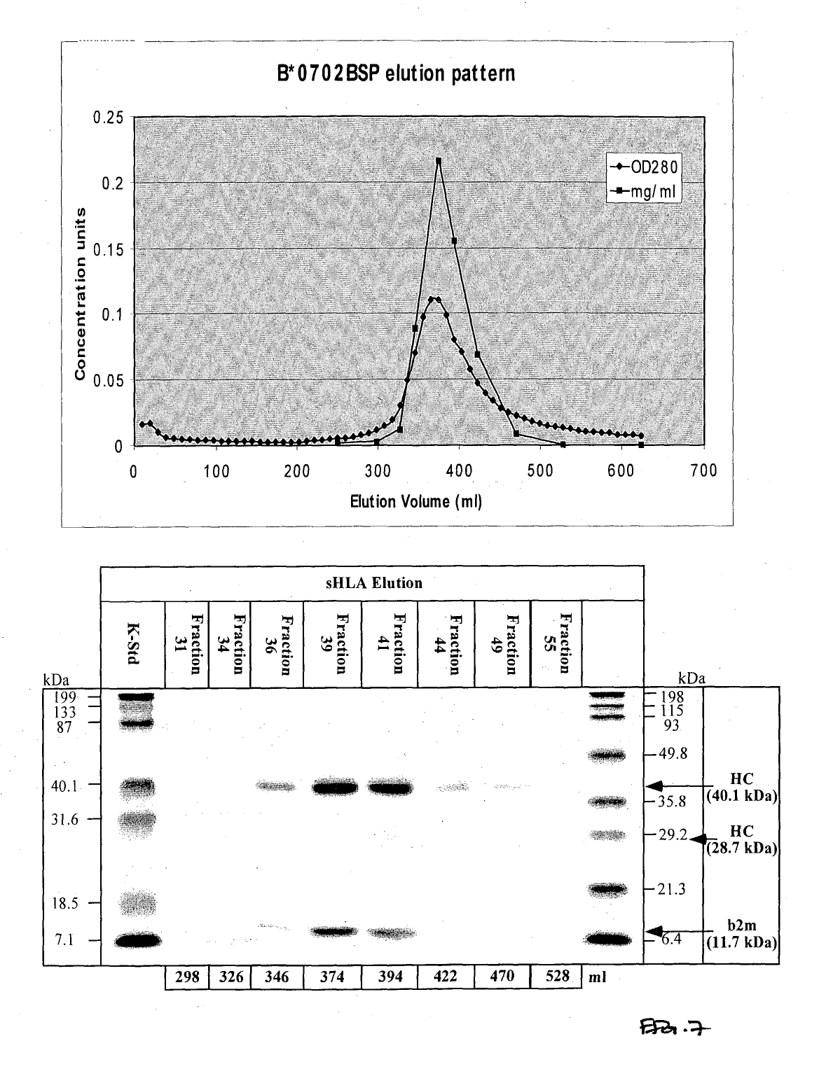

The present invention is directed to a unique method for producing, isolating, and purifying class I molecules in substantial quantities. As an example of the method of the present invention, the following graphs show that the test allele B*0702His produced in static culture can be purified to homogeneity and eluted as intact molecule. FIG. 5 demonstrates that a W6/32-coupled. affinity column can be saturated with crude harvest containing sHLA. Individual values were determined through a standardized sandwich ELISA procedure using W6/32 as capturing antibody and anti-β2m as detecting antibody. This ELISA procedure allows only the detection of intact sHLA molecules. After successful loading, the column is washed with PBS. FIG. 6 shows the washing step. The removal of total protein and active sHLA measured through OD₂₈₀ and ELISA, respectively, can be followed. It shows that after 500 ml of wash volume, impurities are successfully removed from the column. This was also confirmed through SDS-PAGE analysis of the wash fractions collected. In FIG. 7, we were able to elute sHLA molecules with 0.1 M glycine (pH 11.0) and neutralize in 1 M potassium phosphate (pH 7.0) that resulted in fractions of intact molecules as shown through the standard ELISA procedure. Elution occurred in a single peak. indicating the absence of nonspecifically bound material on the column. SDS-PAGE analysis confirmed the size of the subunits and their purity. The final Macrocep procedure was used to remove the neutralization buffer and replace it with PBS (0.02% Sodium azide). Experiments presented hereinafter demonstrate that this buffer is highly suitable to maintain structural integrity and maintain the stability of the sHLA complex.

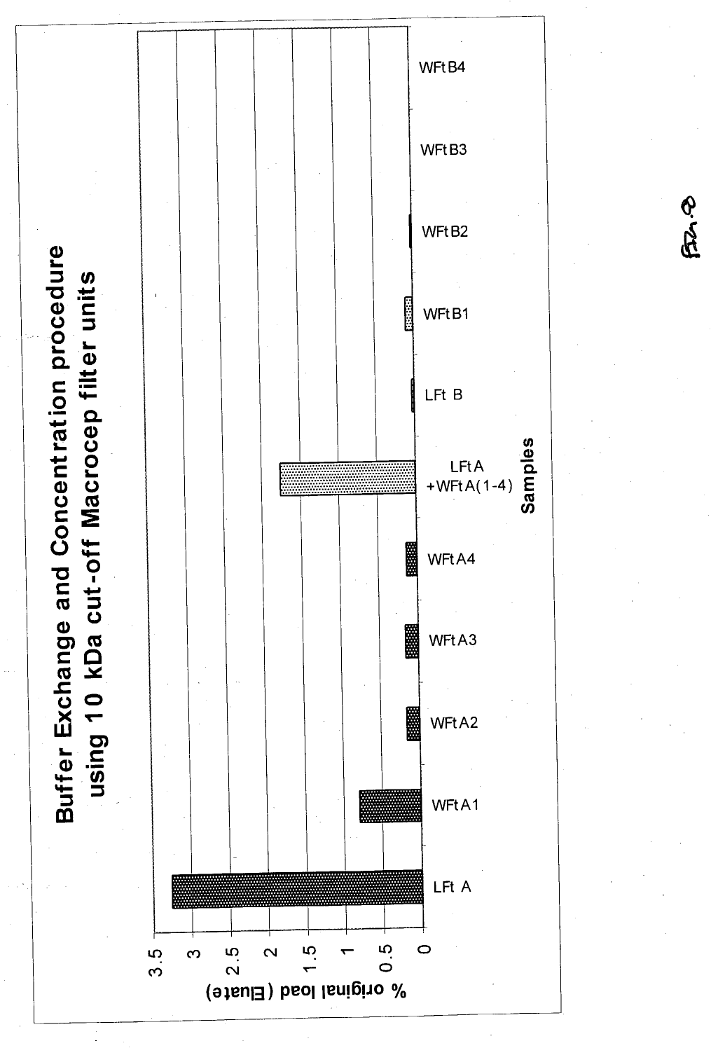

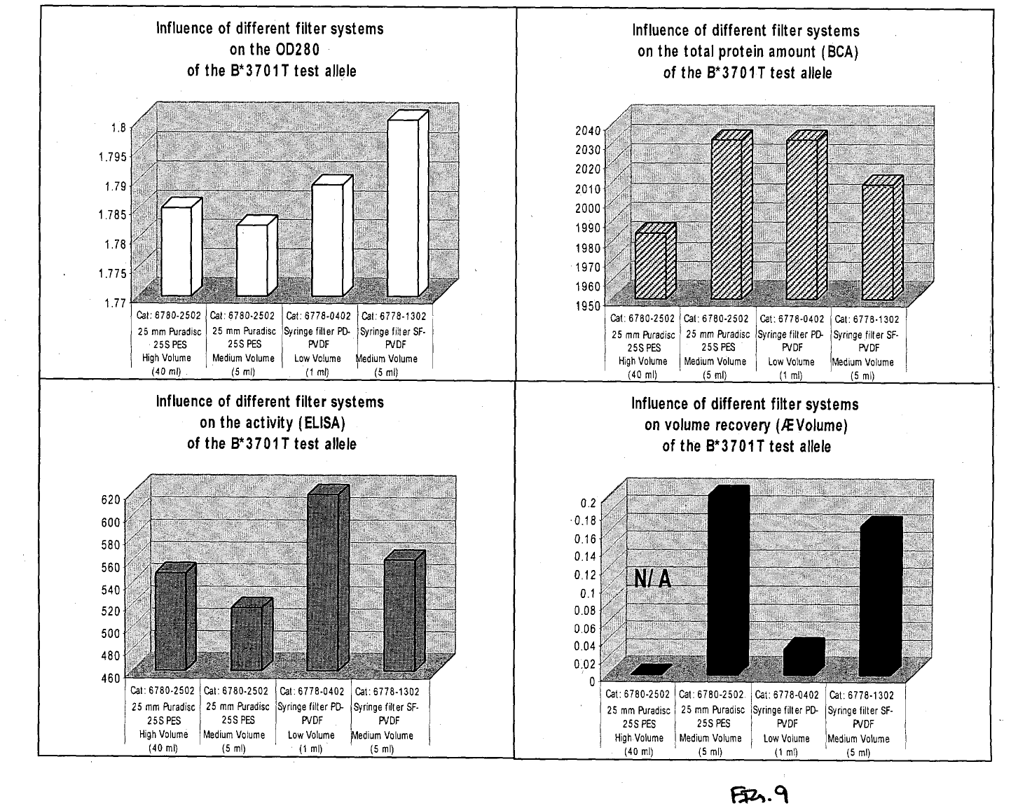

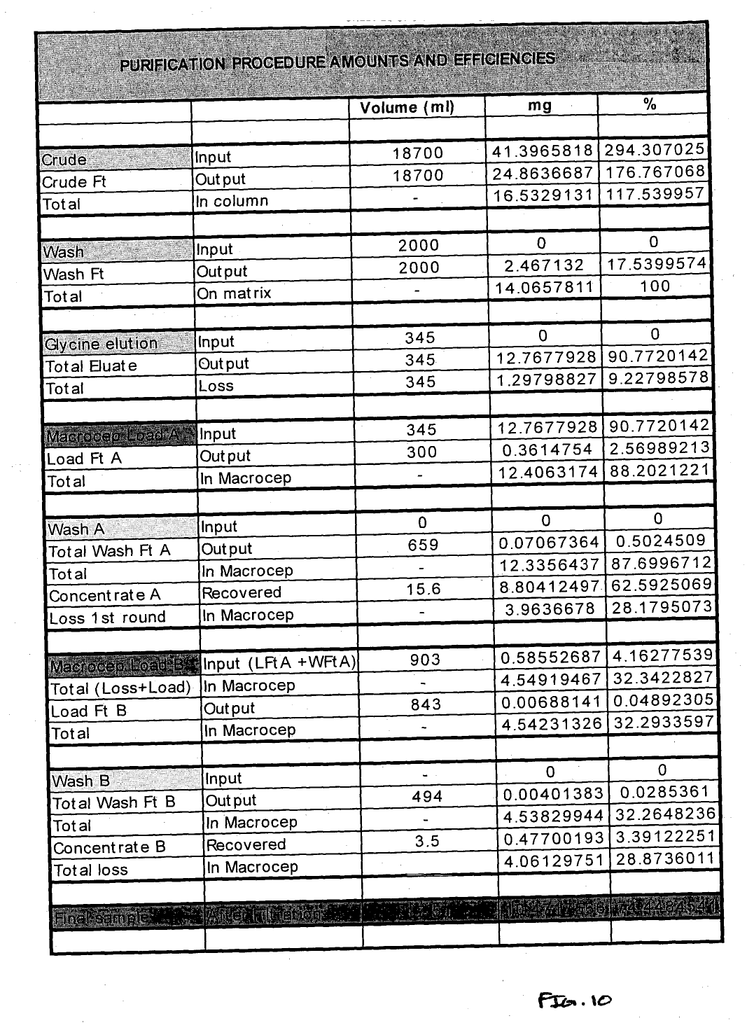

The same procedure is used to finally concentrate the protein to increase the stability of the molecules. Higher concentrations are also more suitable in most applications. FIG. 8 shows two rounds of buffer-exchange and confirms minimal loss of protein after the last step. All wash flow-through's (WFt's) have minimal sHLA content and are usually discarded after the procedure. The sHLA content was elaborated using the standard ELISA technique. To remove possible particles or bacterial growth, filtration through a 0.2 micron filter is standard procedure. FIG. 9 demonstrates that filter-units tested perform nearly equally good and no decline in total protein through absorption to the filters or loss of activity could be detected. The recovery volume was also highly acceptable and only small amounts of liquid did remain within the filters. FIG. 10 shows the efficiency of the procedure measured at each step. A 100% was defined as the sHLA content directly bound to the column after loading and wash. All Flow-through's and washes having substantial amounts of sHLA are recovered and can be reused as loading material for a second round of purification. With this purification run, a total efficiency of 75% was achieved.

Chemical and Physical Purity of Individual, Soluble MHC Molecules

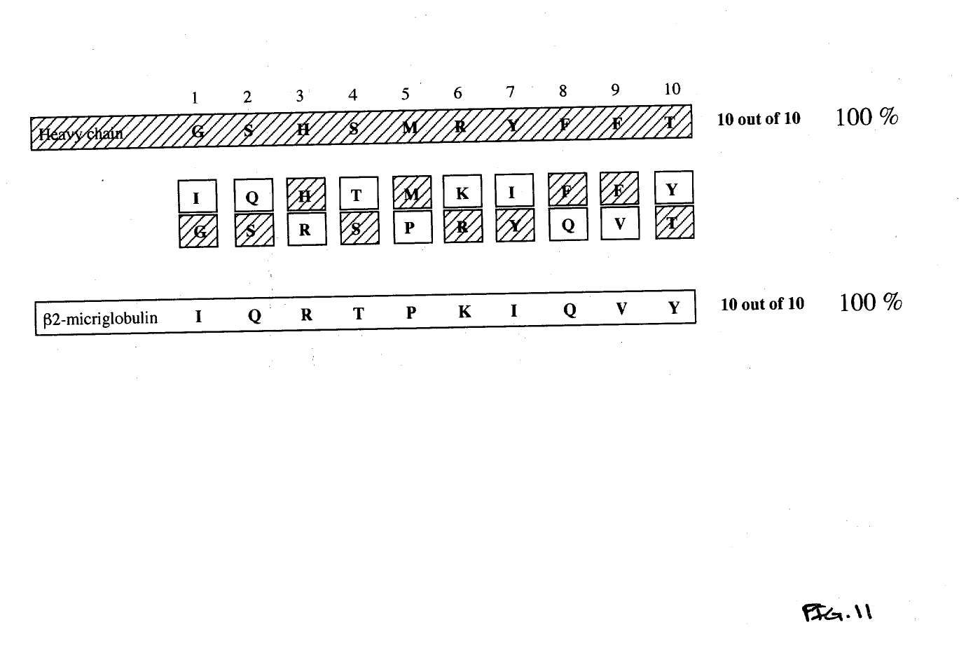

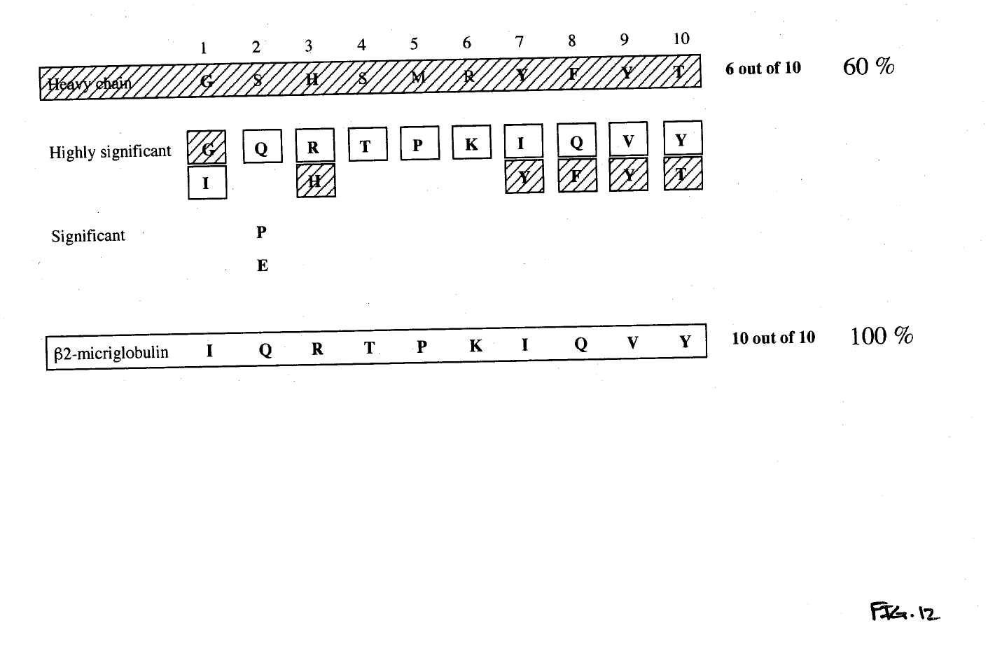

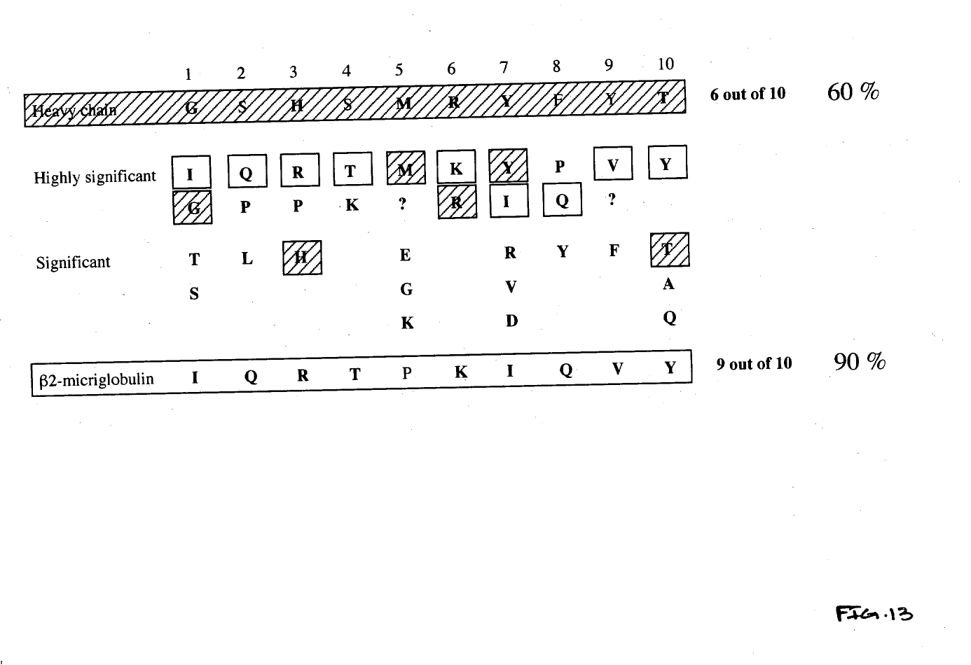

To confirm that the sHLA produced and purified by the method of the present invention are correctly translated, an Edman degradation was performed to receive the sequence of the first 10 amino acids. Since an intact sHLA molecule is a complex consisting of HC, β2m and a peptide, sequencing results gave us several different amino acids at each position. Since HC and β2m are present in a ratio of 1:1 each position from 1 to 10 should predominantly contain both HC and β2m amino acids in about equal amounts. Since both sequences are published and well known, a comparative analysis can easily be done. Because sHLA molecules bind a variety of different peptides, these amino acids are producing noise at each position rather than delivering distinctive recognizable amino acids which makes it in certain cases impossible to make a proper evaluation. Three different molecules were sequenced: FIGS. 11-13 illustrate protein sequence data for MHC Class I HLA-A*0201T, HLA-B*0702T, and HLA-B*1512T, respectively. The comparison clearly shows that the sHLA's are correctly translated at the amino terminal end. It is also evidence that no other major impurity was present in those samples.

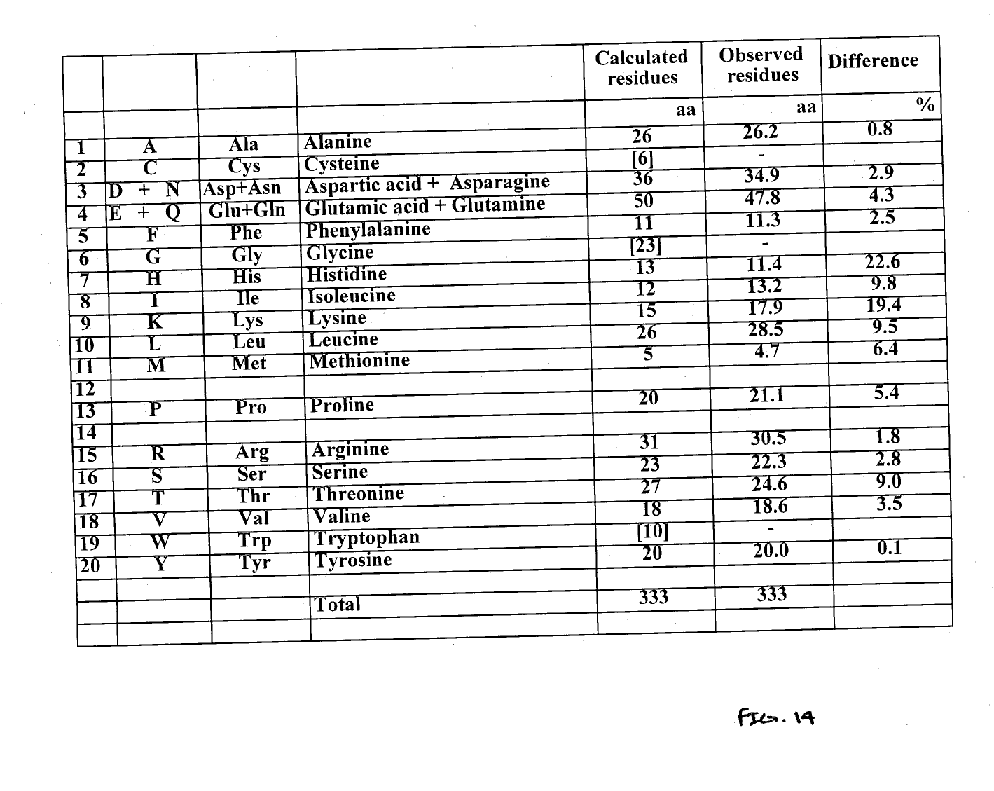

Proteolysis of the whole molecule complex and analysis of the amino acid composition was executed on the B*1512T (FIG. 14). The procedure showed a close relationship between the amino acid content of the calculated versus the observed residues suggesting a full length molecule. During the procedure, some amino acids were expectedly degraded and were not taken into consideration. The close match is a further indication of the purity of our test-sample.

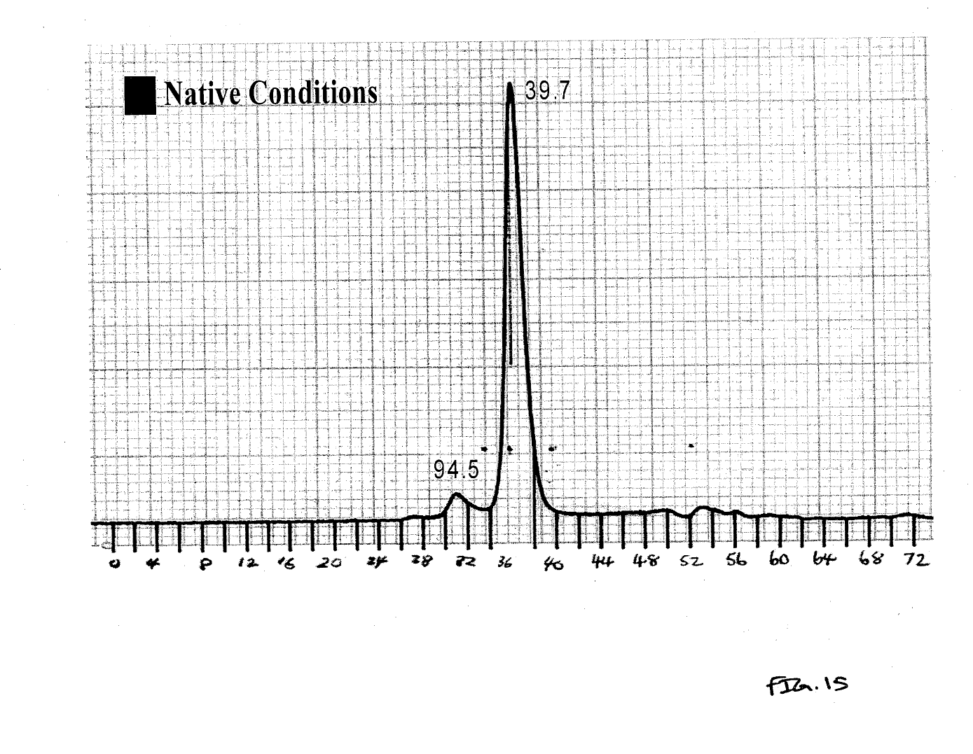

The sHLA's produced and purified by the method of the present invention were analyzed by Superdex chromatography to demonstrate sample purity (FIG. 15). The Superdex-FPLC analysis under native conditions for B*1512T showed a characteristic peak corresponding to the sHLA complex. No other major bands can be detected confirming the pure nature of our preparation. Under such native conditions, a peak of the size of 39.7 kDa is seen, which is in the area of complexed sHLA. No bands at 31 kDa, representing free HC, or at 12 kDa for β2m are visible. However, a minor band at approximately 94.5 kDa can be seen, which represent aggregated HCs. Because sHLA samples are filtered through a 10 kDa filter during the Macrocep procedure, these free HC molecules remain in the solution and cannot be removed. Aggregated HC molecules are not considered an impurity of the sample. Their contribution to the final protein amount is less than 1%. The overall purity of the complex compared to foreign proteins is more than 99.9%.

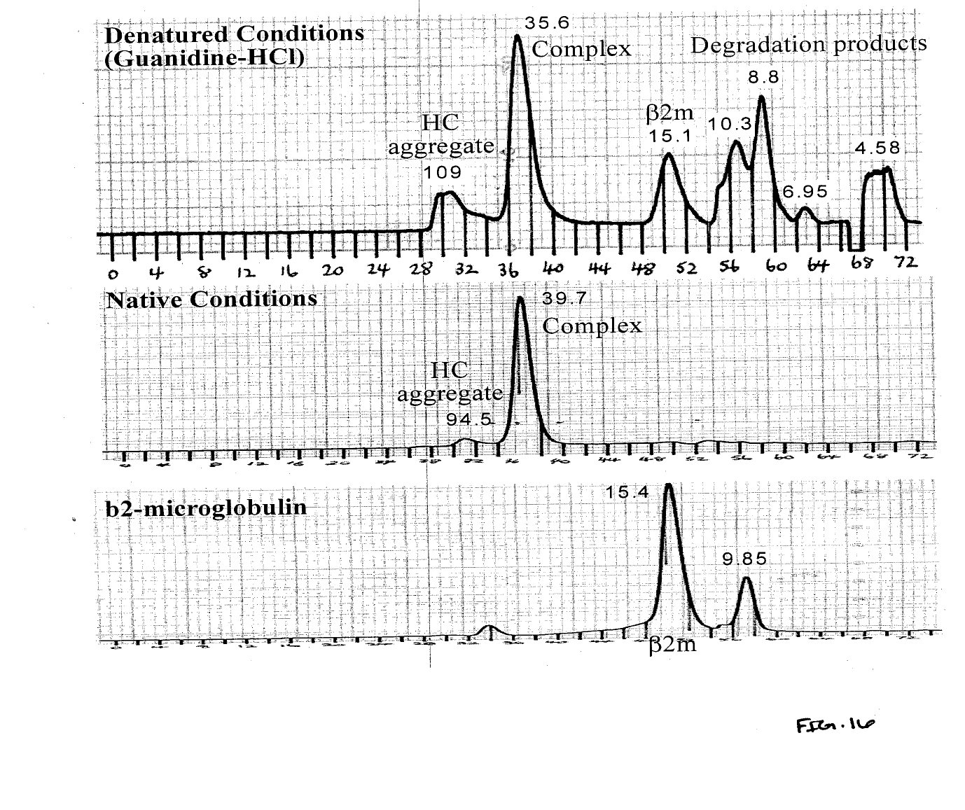

A triple analysis of B*1512T is presented in FIG. 16. It shows a separation of sHLA under denaturating and under native condition as well as separation of purified free β2m (Serotec) alone. A standard curve was run in parallel to estimate molecular weights.

Using guanidine-HCl as additive to denature the probe, the sample of B*1512T was run under equal conditions as the other samples. The results seen demonstrate that the sHLA complex is unexpectedly stable under such denaturing conditions. A clear peak resembling the pure complex can be identified which is at the same position as the native peak. As expected, sHLA complexes do fall apart, which resulted in the increase of aggregated HC and an increase in free β2m as their positions are identified through their overlap with the native samples. Surprisingly, the denaturation process did not deliver a peak at 31 kDa corresponding to free HC. It seems that HC monomers are not present and immediately aggregate to a higher size complex. During the denaturing process, several peaks of lower molecular weight appeared, which correspond not only to aggregated peptides released from the destroyed complex but also through fragmentation of β2m and HC subunits.

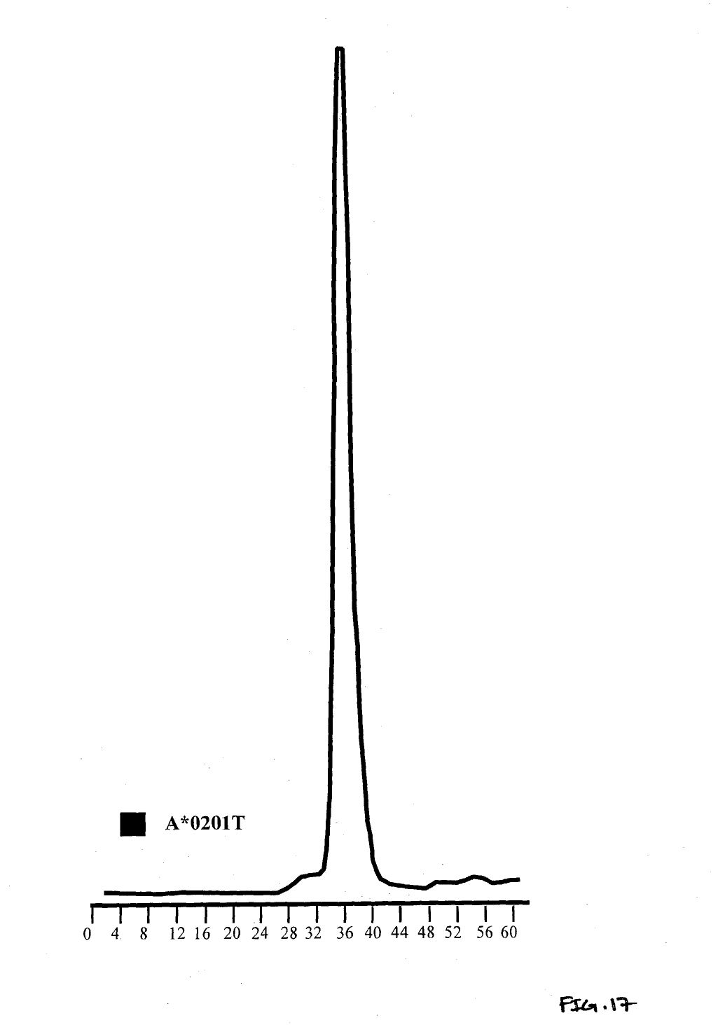

The results of purity are not a unique event and can be demonstrated with all alleles going through our optimized purification procedure. A Superdex profile of A*0201T is provided as an additional example in FIG. 17.

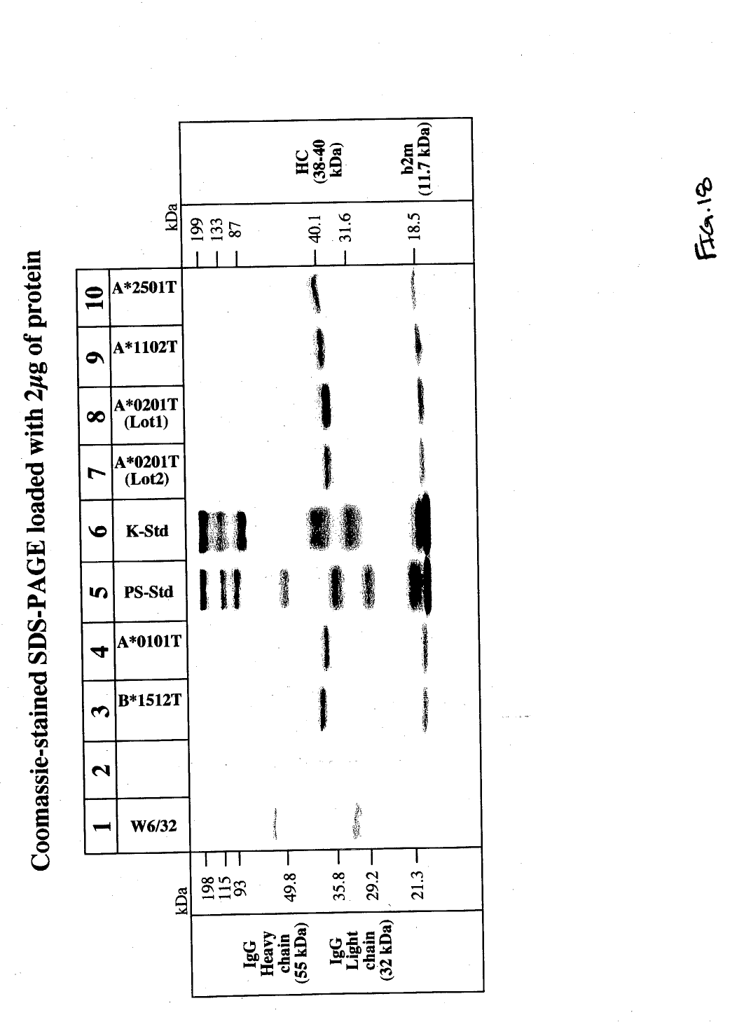

Several sHLA alleles were loaded on an SDS-PAGE gel and stained with Coomassie to assess the purity of the samples (FIG. 18). A band for HC and β2m, respectively, Was detected demonstrating the purity of all samples tested. The antibody W6/32, which is used in the process of affinity purification, is also added. In none of the samples could an equal band be detected, thus showing that leakage of W6/32 during elution does not occur.

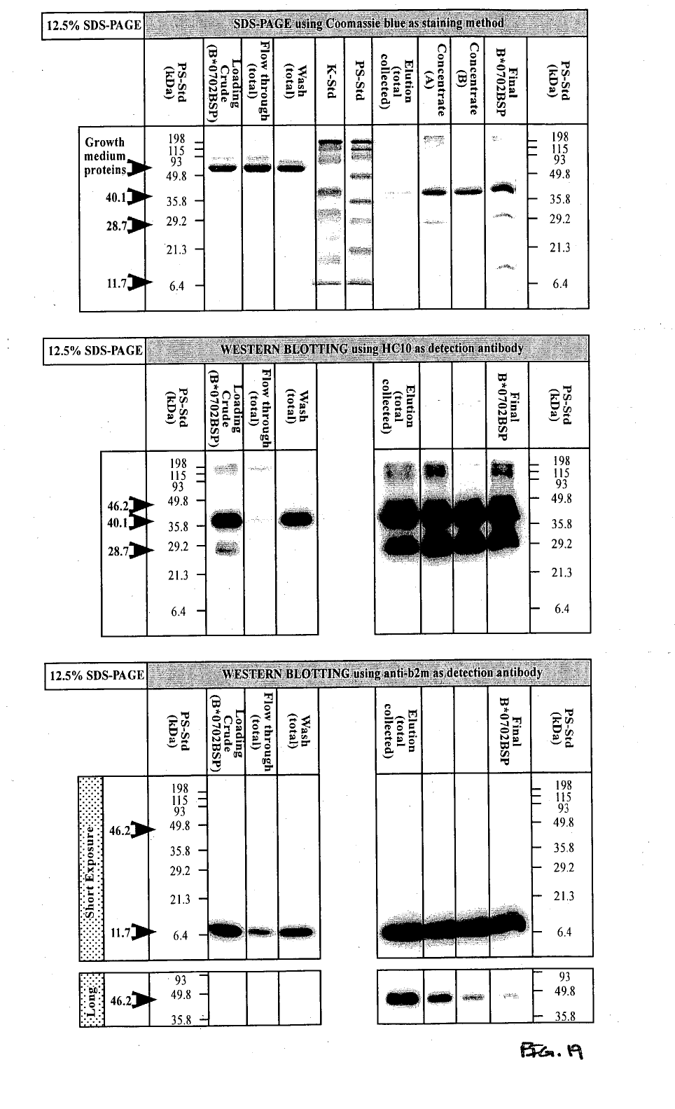

Western blot analysis to follow the HC and β2m subunits of sHLA were also performed (FIG. 19). The upper portion shows the results of an SDS-electrophoresis performed running crude harvest (load), the flow through (output of the column) and the wash on the left side, eluate, concentrate and final sample on the right.

Using HC10 antibody visualized with a secondary mouse antibody coupled to HRP, several bands could be stained resembling different aggregates of HC. It appears that the dimeric form is dominant (40.1 kDa) over the monomeric form (28.7 kDa) after denaturation and SDS treatment. The lower value for the dimeric form is evidently an artifact and caused by an aberrant running behavior on SDS-PAGE gels since a consistent amount of SDS is not anymore bound per unit weight of protein. The carbohydrate moiety attached to the HC might also be involved. Higher aggregates are also visual to a minor extent. The results show that sHLA is present in the crude and binds to the column since there is a drastic reduction in signal observed in the flow through. Saturation of the column does result in material leaving the column not captured. Therefore, wash fractions will also contain some sHLA not captured. The protein is highly concentrated in the purified sample and concentrates do not look different than eluted molecules.

An anti-β2m antibody directly labeled with HRP was used to visualize the lighter subunit. A single band of 11.7 kDa was seen as expected. β2m does not seem to aggregate. However, a faint band at 46.2 kDa could be observed. An extended exposure showed a clear band at this location which is in the size of the intact complex. This would suggest that some complexes survived the denaturation step and show SDS resistance.

Separation under denaturing conditions and staining with the antibodies HC10 and anti-β2m revealed that both the heavy chain and β2m are present. The secondary antibody directed against mouse antibodies also did not reveal any additional bands, indicating that the preparation is free of possible W6/32 antibody contamination, which was used in the purification step.

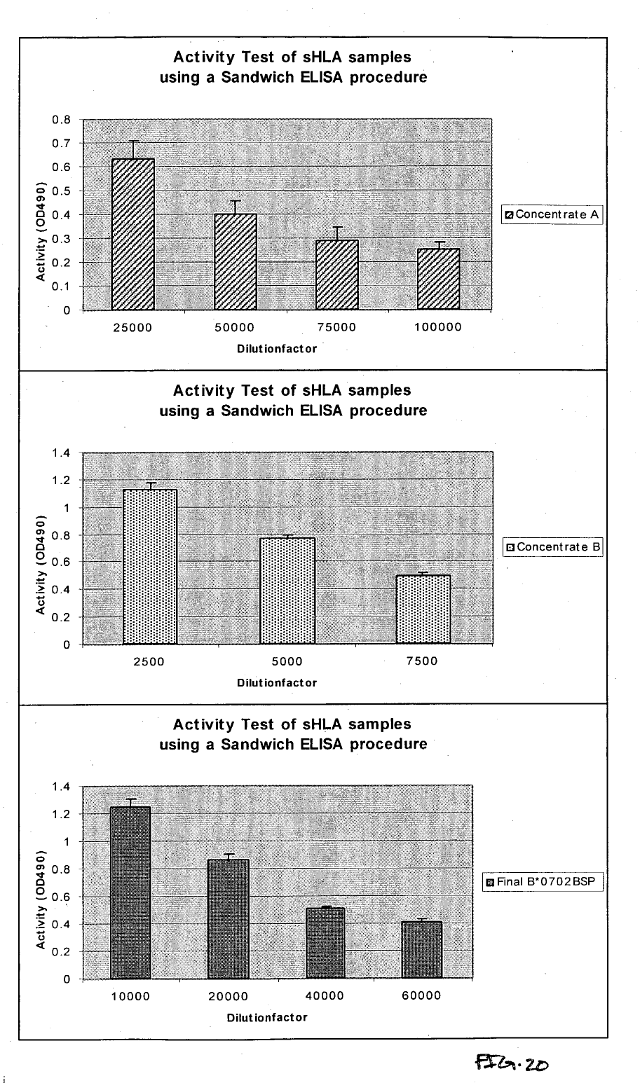

The Sandwich ELISA procedure was used to follow the sHLA molecule through all purification steps and confirm activity of the sHLA molecule (FIG. 20). Final analysis confirms that at no time did the sHLA molecules denature and that the sHLA molecules always maintain their structural integrity. Activity can still be detected in highly diluted samples.

Functional Purity of Individual, Soluble MHC Molecules

1. Conformation-Specific Antibody Binding Assays

The use of Pan class I antibodies gives conclusive results about the conformational status of the sHLA molecules. Thus, sHLA activity tests using Pan-class I antibodies such as W6/32, TP25.99, and Pan class I (One Lambda) were performed. W6/32 only recognizes conformationally intact molecules; TP25.99 recognizes the complexed sHLA molecule as well as free HC and the Pan class I (One Lambda) which has equal recognition patterns as seen with W6/32. The antibody HC10 is useful in distinguishing free from bound heavy chain (HC) since this antibody only recognizes the HC of denatured sHLA molecules. Anti-β2m recognizes the β2m subunit in both cases, complexed to the HC as well as free in solution and gives complementary information in addition to the other antibodies.

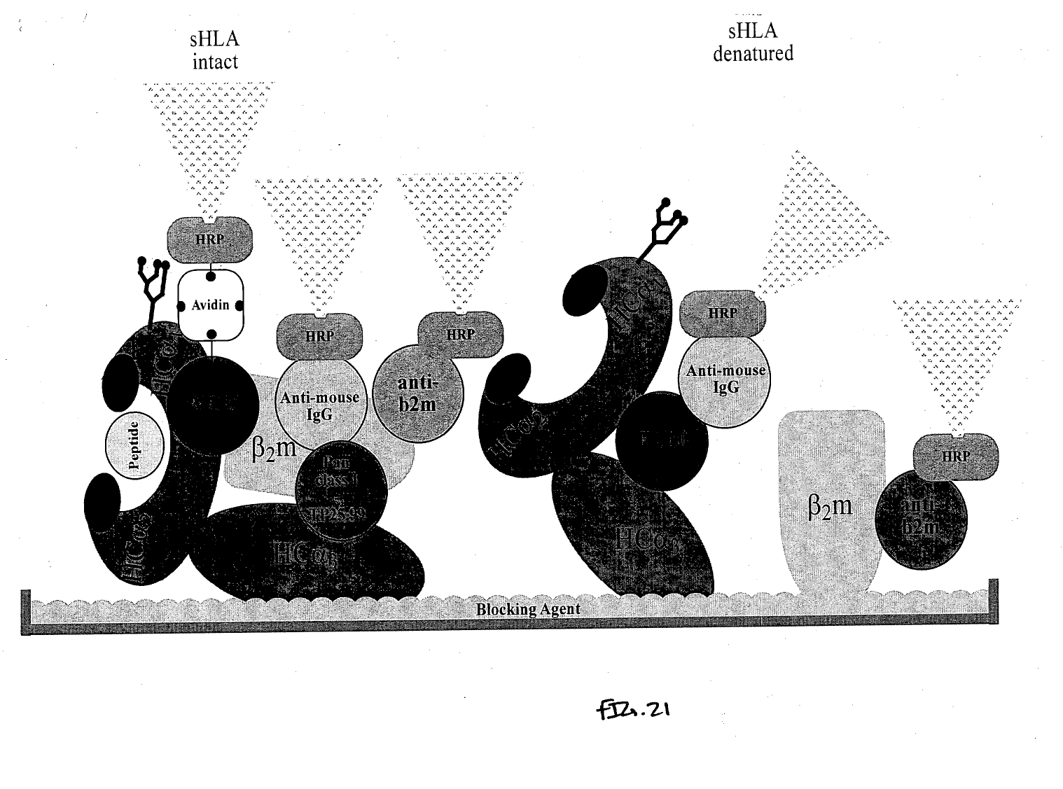

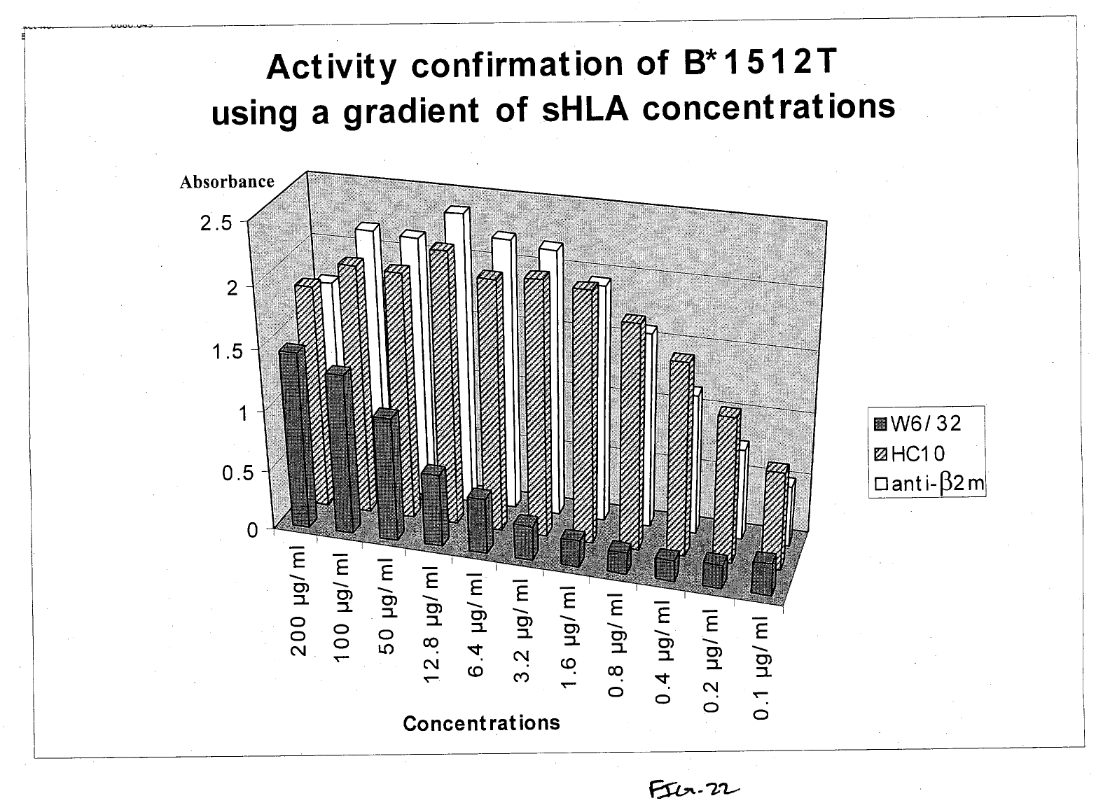

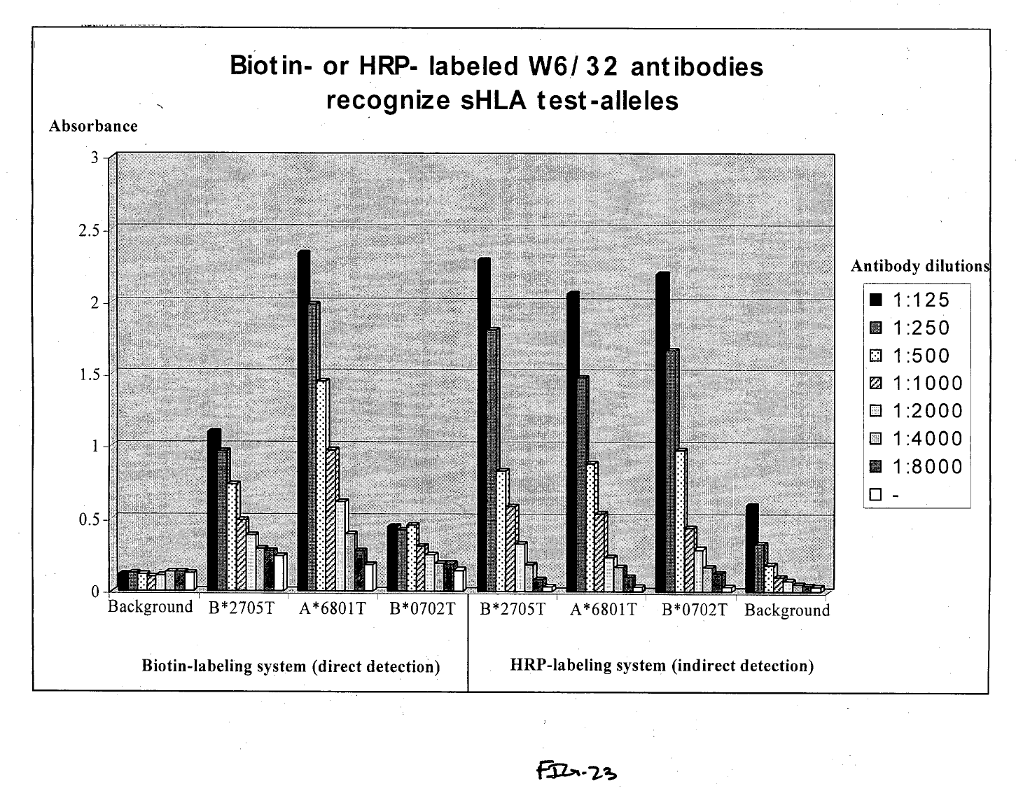

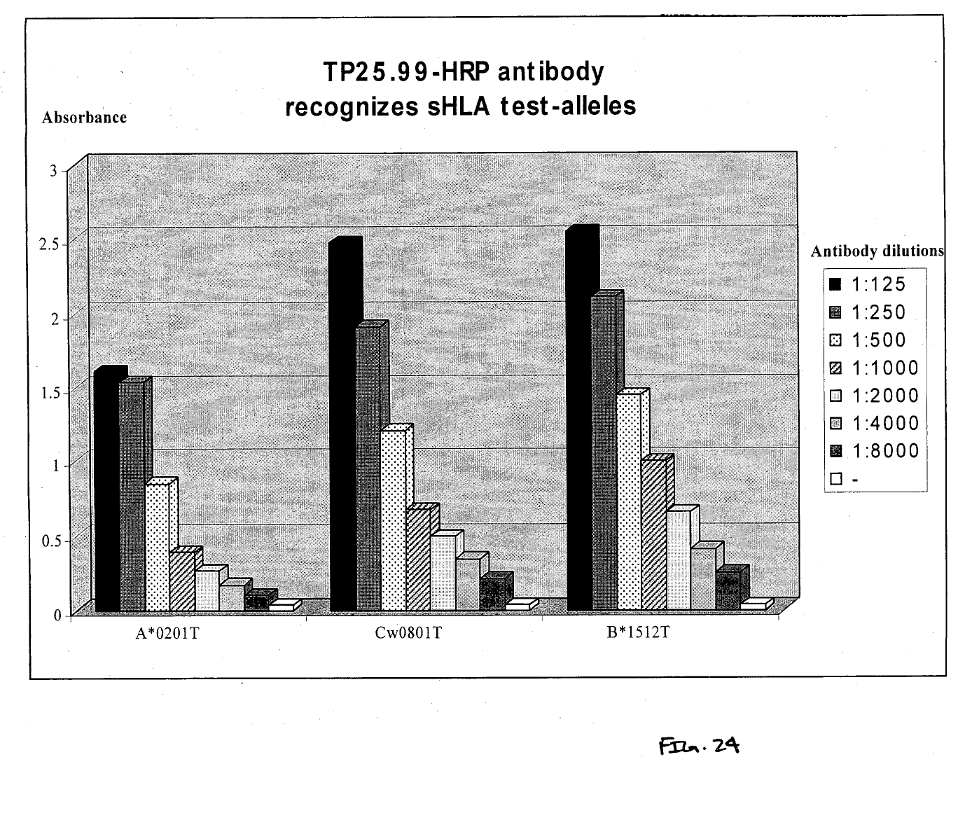

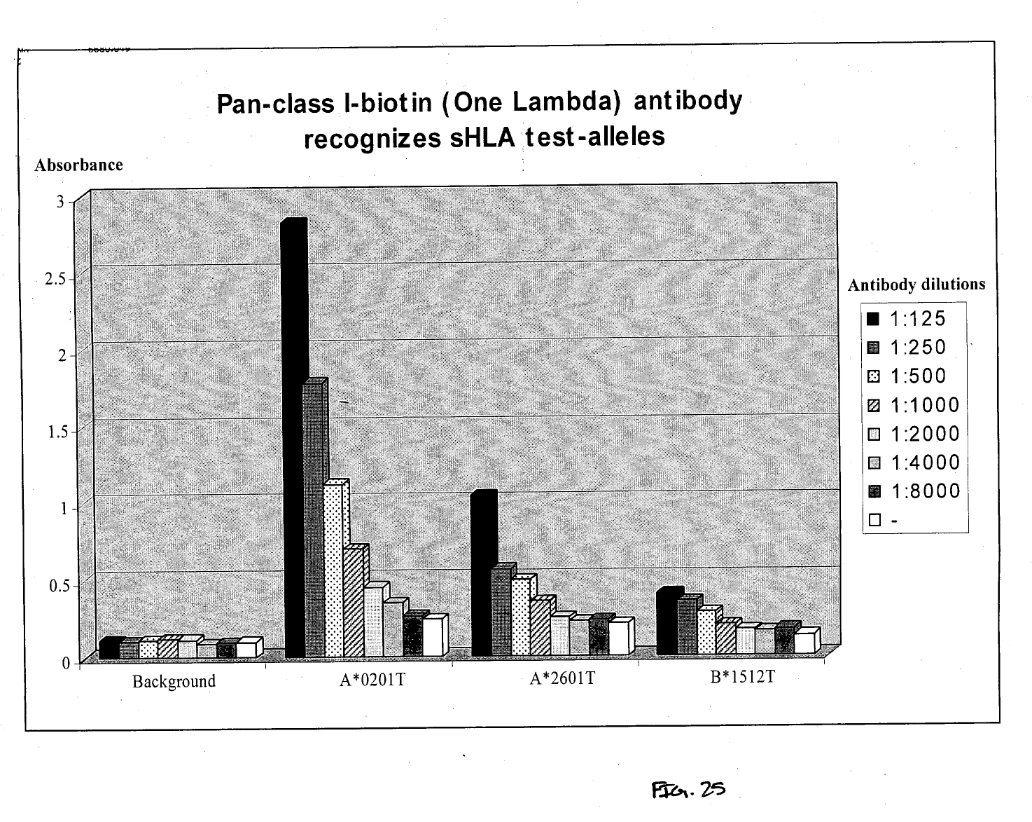

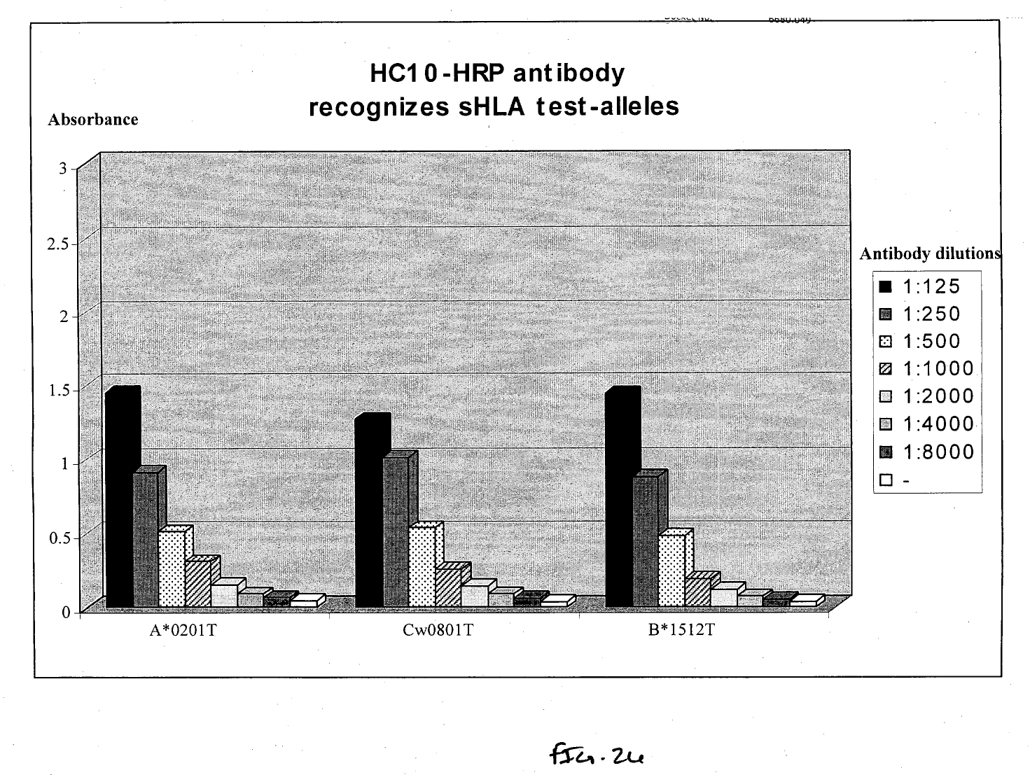

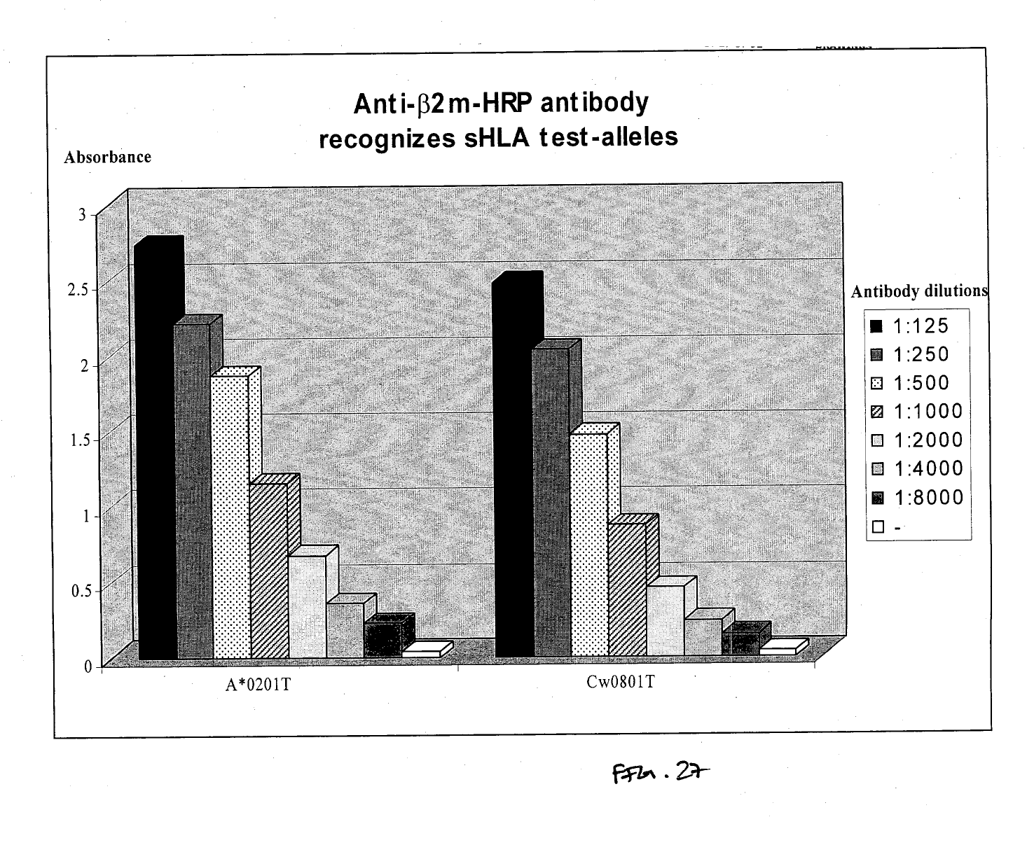

Illustrated in FIG. 21 is a scheme of antibody binding scenarios, while FIGS. 22-27 each illustrate reaction panels for conformation-specific Ab binding assays using Sandwich ELISA assays. The Sandwich ELISA assays include six steps: (1) choice of appropriate support; (2) coating with pan HLA specific antibodies; (3) blocking procedure to reduce non-specific protein binding; (4) capturing of single specificity sHLA molecules at different epitopes; (5) positive (or negative) SERA binding to presented sHLA alleles; and (6) detection of reactive SERA antibodies using secondary anti-human IgG (IgM) antibody.

Sandwich assays can be used to study a number of aspects of protein complexes. If antibodies are available to different components of a heteropolymer, a two-antibody assay can be designed to test for the presence of the complex. Using a variation of these assays, monoclonal antibodies can be used to test whether a given antigen is multimeric. If the same monoclonal antibody is used for both the solid phase and the label, monomeric antigens cannot be detected. Such combinations, however, may detect multimeric forms of the antigen. The W6/32-anti-β2m antibody sandwich assay is one of the best techniques for determining the presence and quantity of sHLA. Two antibody sandwich assays are quick and accurate, and if a source of pure antigen is available, the assay can be used to determine the absolute amounts of antigen in unknown samples. The assay requires two antibodies that bind to non-overlapping epitopes on the antigen. This assay is particularly useful to study a number of aspects of protein complexes.

To detect the antigen (sHLA), the wells of microtiter plates are coated with the specific (capture) antibody W6/32 followed by the incubation with test solutions containing antigen. Unbound antigen is washed out and a different antigen-specific antibody (anti-β2m) conjugated to HRP is added, followed by another incubation. Unbound conjugate is washed out and substrate is added. After another incubation, the degree of substrate hydrolysis is measured. The amount of substrate hydrolyzed is proportional to the amount of antigen in the test solution.

The major advantages of this technique are that the antigen does not need to be purified prior to use and that the assays are very specific. The sensitivity of the assay depends on four factors: (1) the number of capture antibodies; (2) the avidity of the capture antibody for the antigen; (3) the avidity of the second antibody for the antigen; and (4) the specific activity of the labeled second antibody.

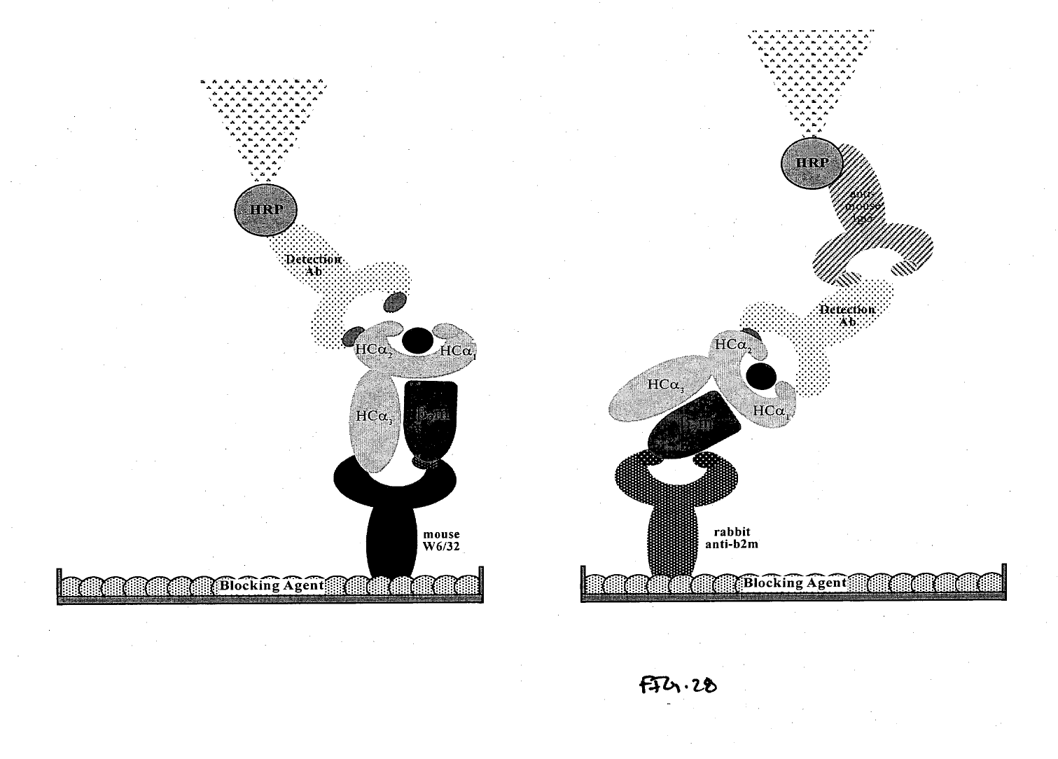

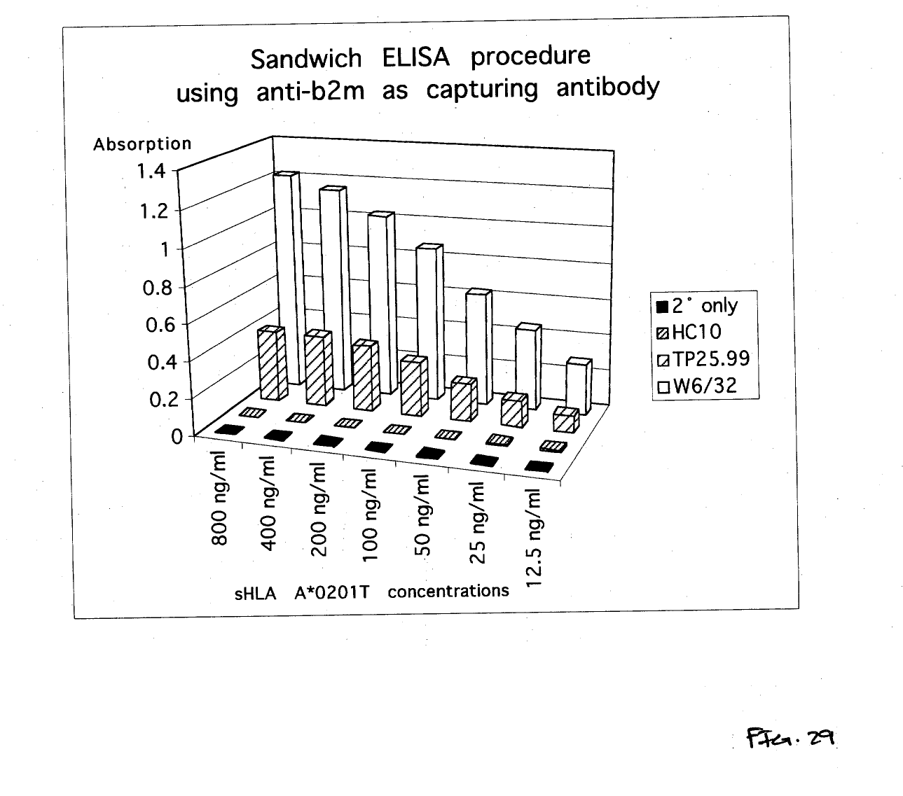

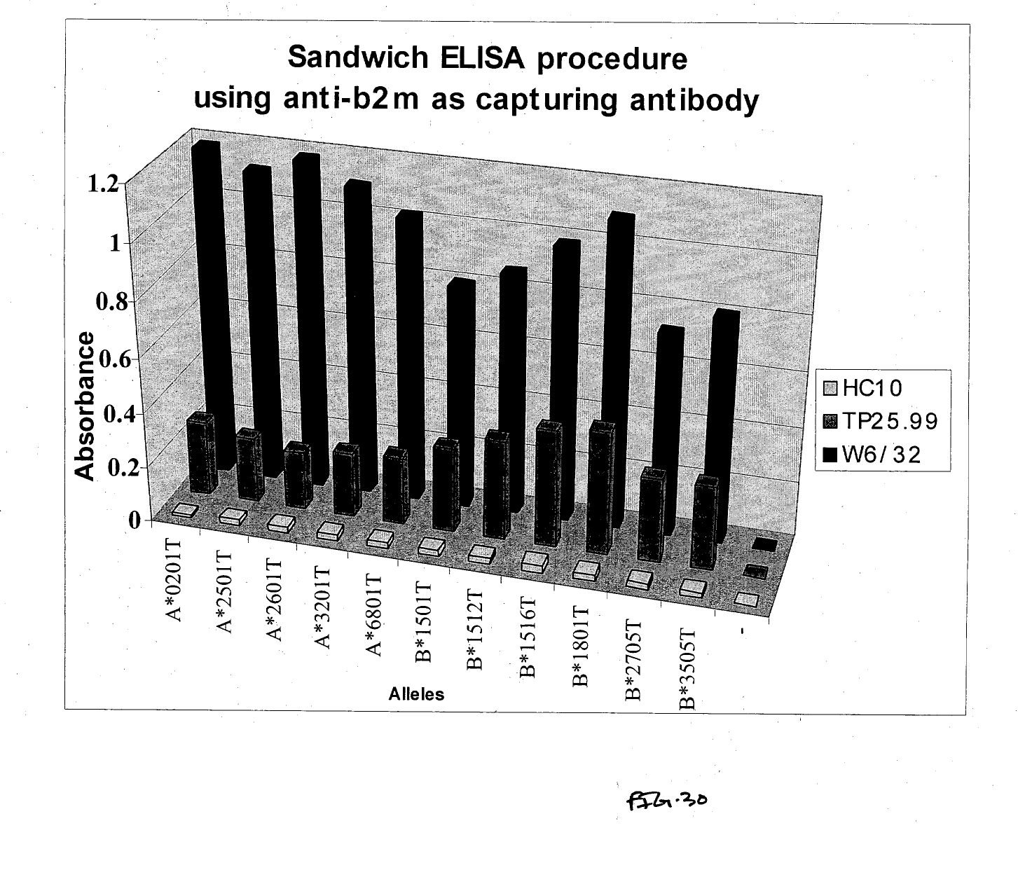

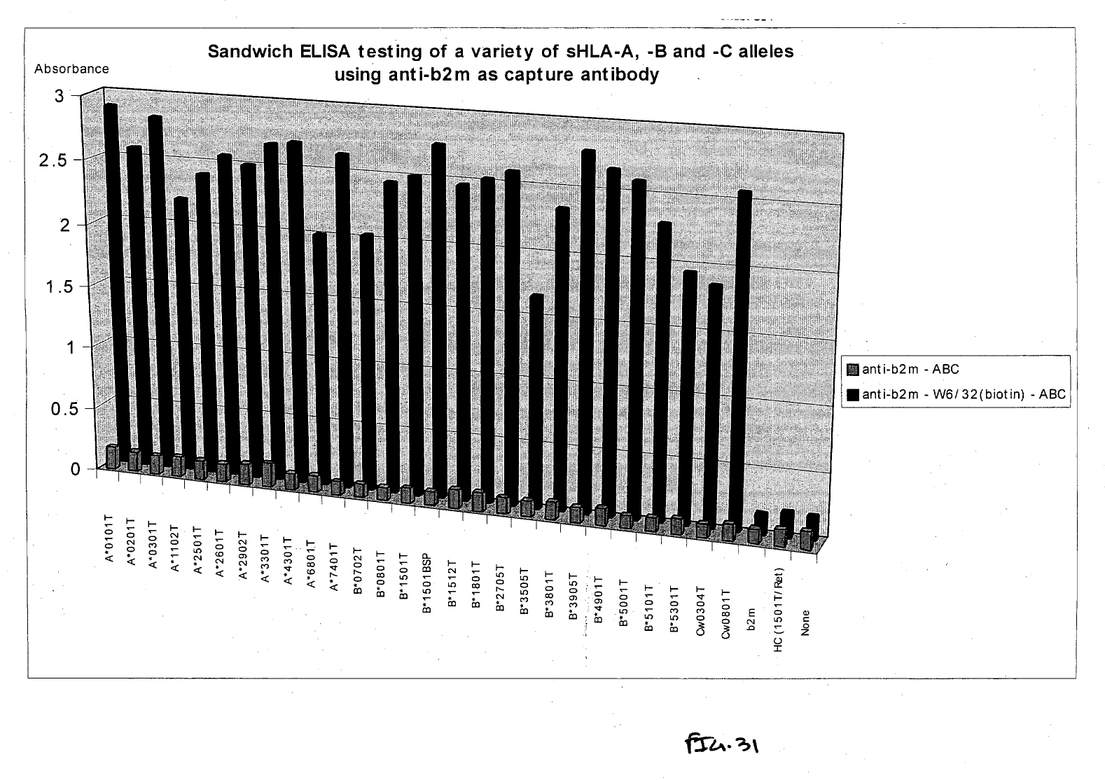

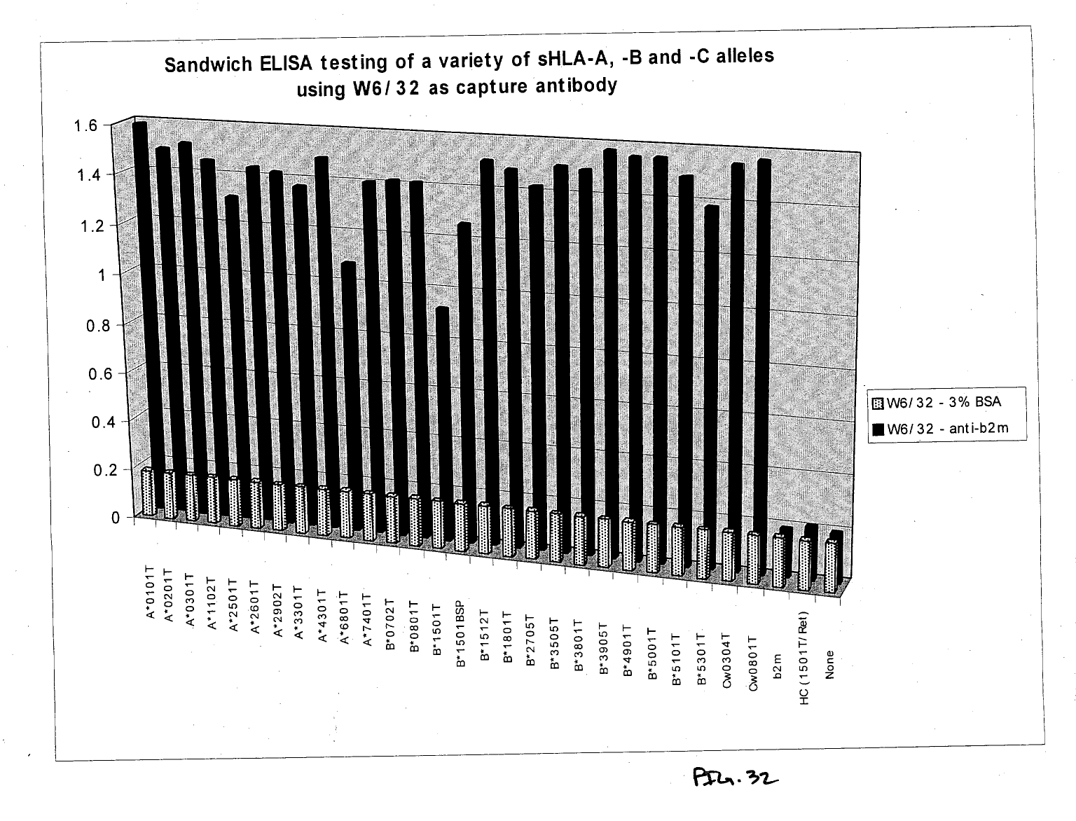

In order to demonstrate proper conformation of our produced sHLA class I proteins, several Pan-class I monoclonal antibodies were tested. Utilizing the sandwich ELISA technique, a selection of sHLA-A and -B alleles captured with anti-β2m or W6/32 were visualized by a variety of detector antibodies specific for sHLA as seen in the scheme of FIG. 28. All results were confirmed by both assay procedures indicating that antigenic integrity of purified sHLA molecules is not compromised. HC10 reactivity was not detected as expected since free HC cannot be captured by anti-β2m or W6/32 (FIGS. 29-32).

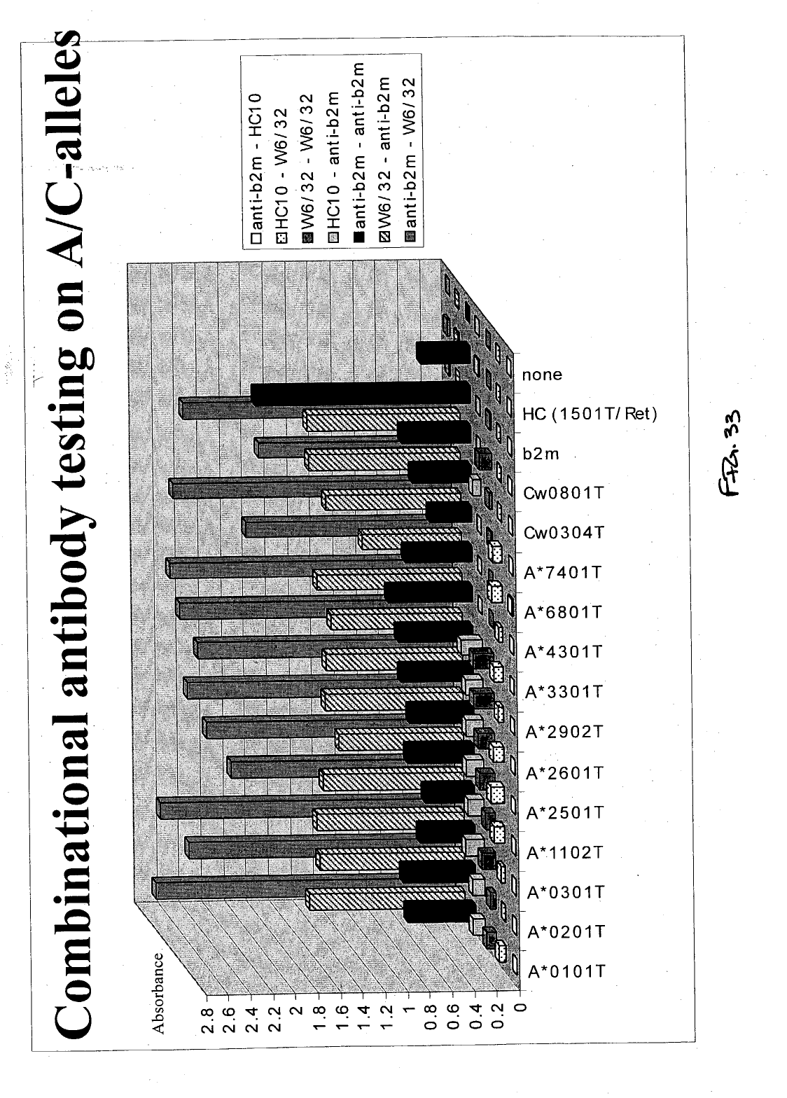

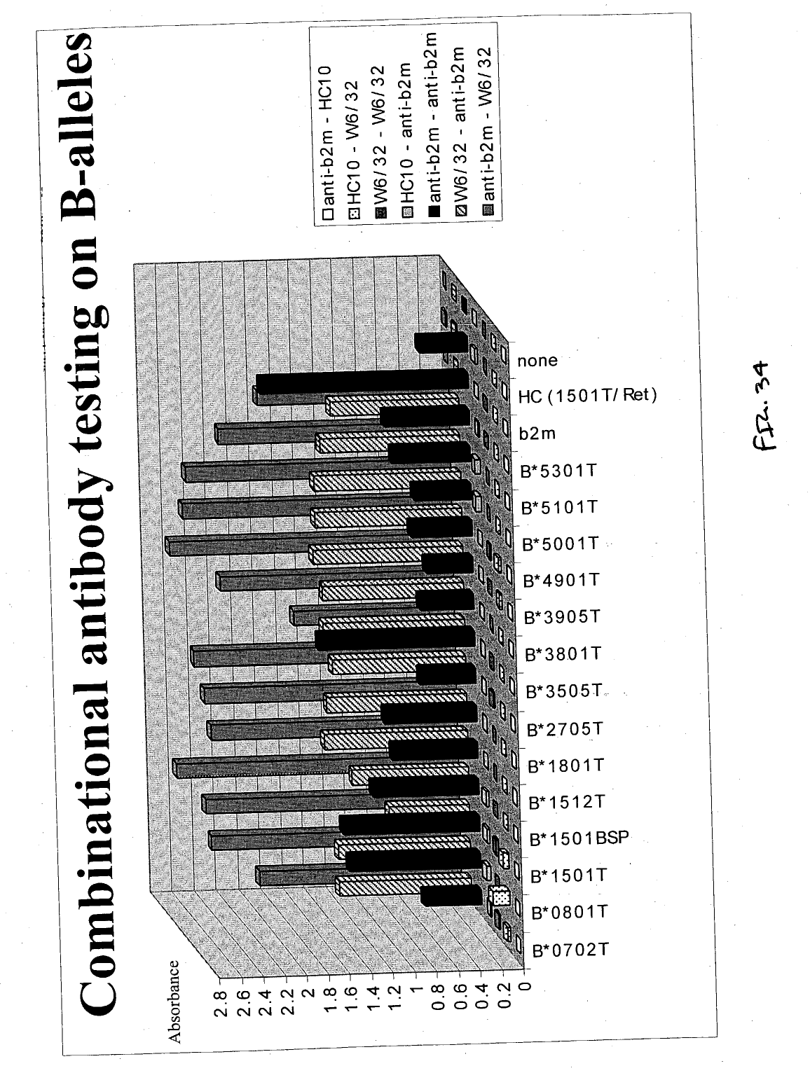

To test for artificial structural forms such as aggregation or dimeric structures, various antibody combinations were tested (FIGS. 33-34). None of these experiments revealed any other structures than single complexes. These complexes have been shown before in equilibrium with very low amounts of free β2m, HC and endogenous peptides.

2. Neutralization Experiments

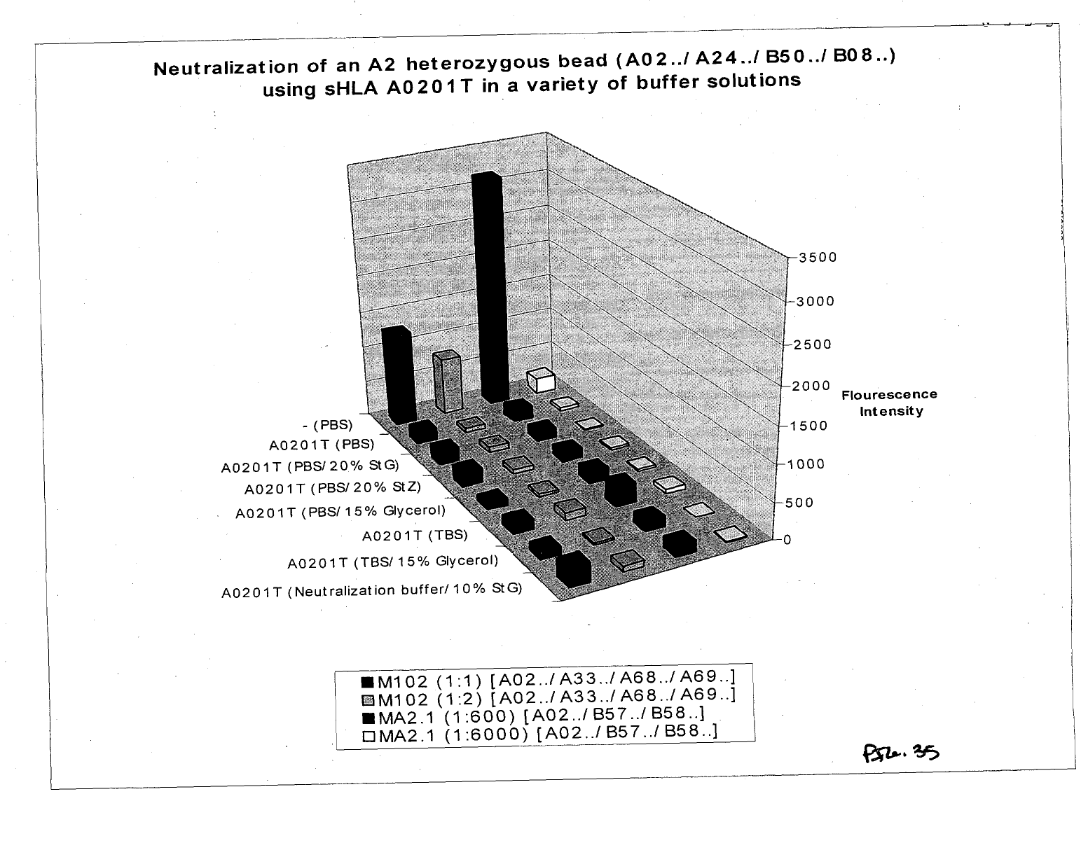

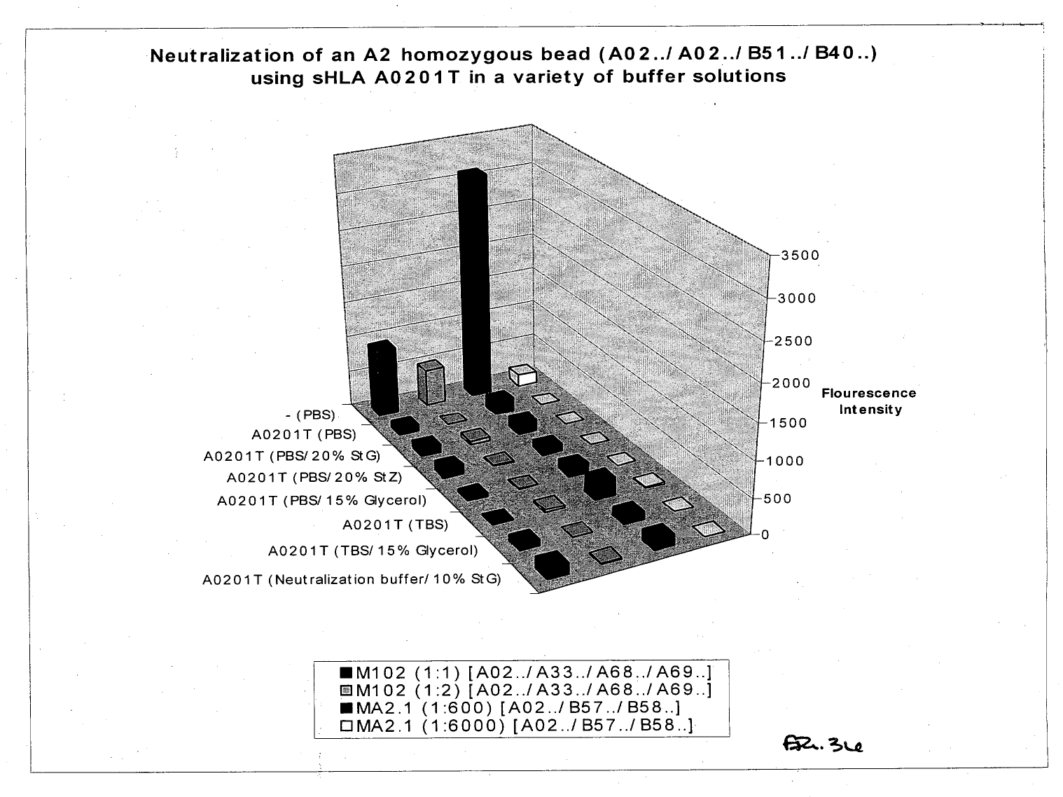

Antigenic integrity was also verified in neutralization experiments (FIGS. 35-36). An established reaction of native beads coupled to HLA molecules interacting with specific human sera could be inhibited by addition of purified sHLA in various buffers which competed for the sera. Different native molecules coupled to beads could be equally neutralized.

The experiments shown in FIGS. 35-36 demonstrate that the sHLA molecule A0201T highly competes with the A2 alloantiserum M102 as well as with the monoclonal Ab MA2.1 confirming the correct behavior of the molecule in this neutralization experiment. This indicates the presence of a native conformationally correct molecule within the samples. Particularly, the MA2.1 (1:600) monoclonal Ab recognizing specific epitopes on A0201T was 93% blocked. Different buffer supplements do not appear to have any influence on the capability to block. The recognition by conformation-sensitive mAbs indicates that the recombinant complex contains native epitopes, consistent with the presence of a correctly folded molecular complex.

3. Chaperone Interaction Experiments

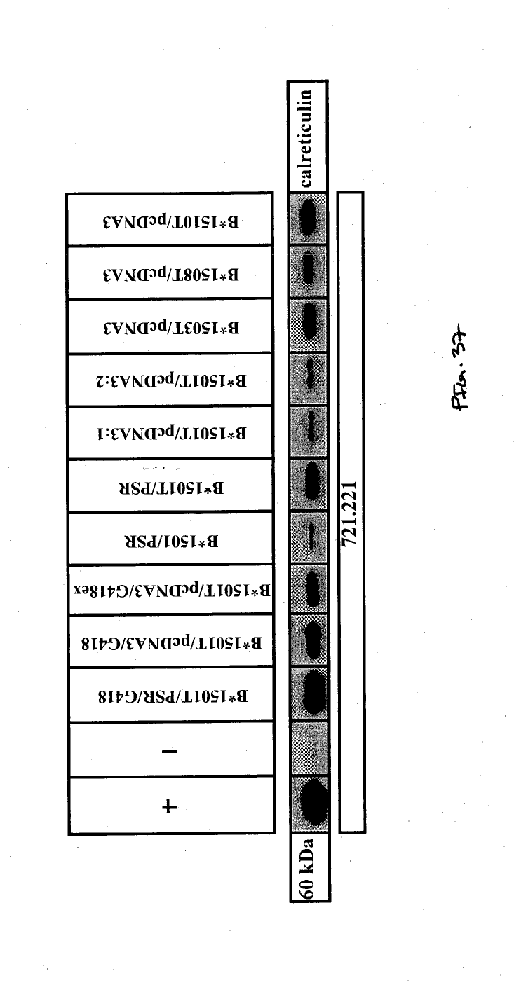

The class I molecule interacts with several chaperones as it traffics through the cell on its way to the cell surface. These chaperones include, but are not limited to, calnexin, calreticulin, Tapasin, and Erp 94. ³⁵S pulse chase/immunoprecipitation experiments were performed to demonstrate that the sHLA class I proteins produced and purified by the method of the present invention interact with chaperones normally. Interaction with calreticulin, calnexin, and tapasin has been demonstrated, and interaction with calreticulin is shown in FIG. 37.

In addition, several experiments have been performed which demonstrate that truncating the HLA molecules does not alter the class I protein. It will be demonstrated herein that the sHLA class I proteins produced and purified by the methods of the present invention interact normally with antibodies specific for the native class I molecule and with peptide ligands.

4. Ab Binding Assays—Single Specificity Antibodies

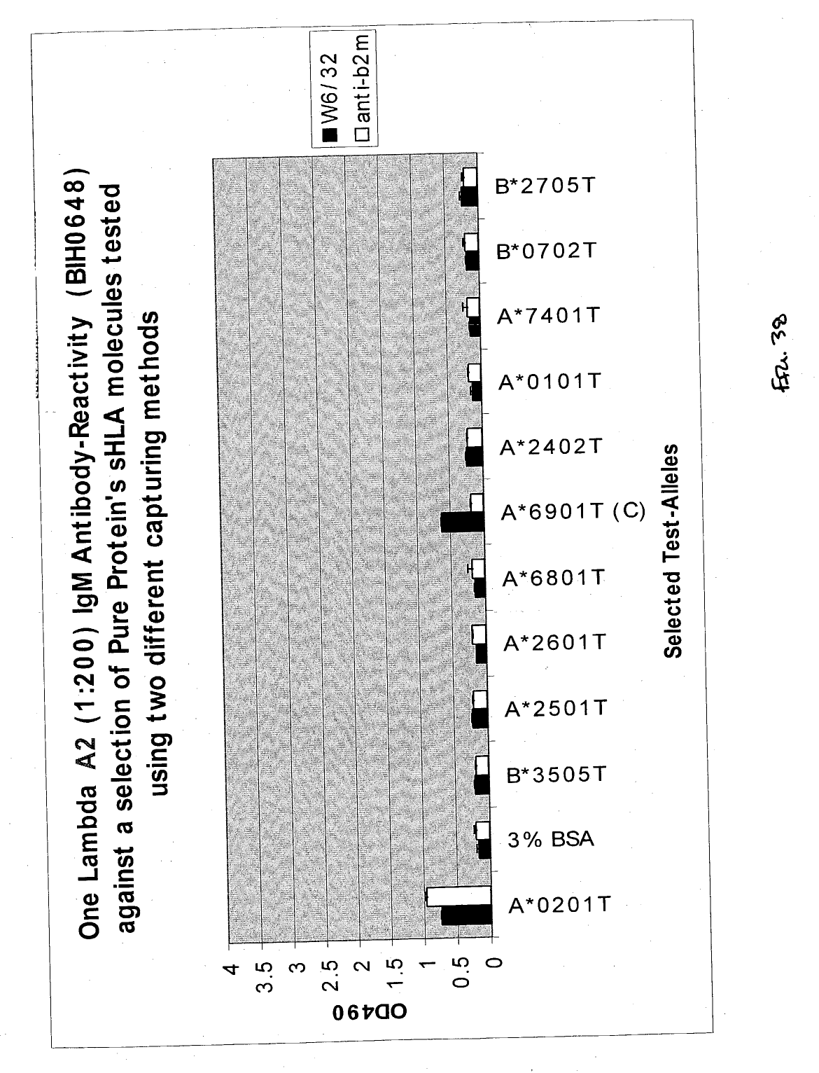

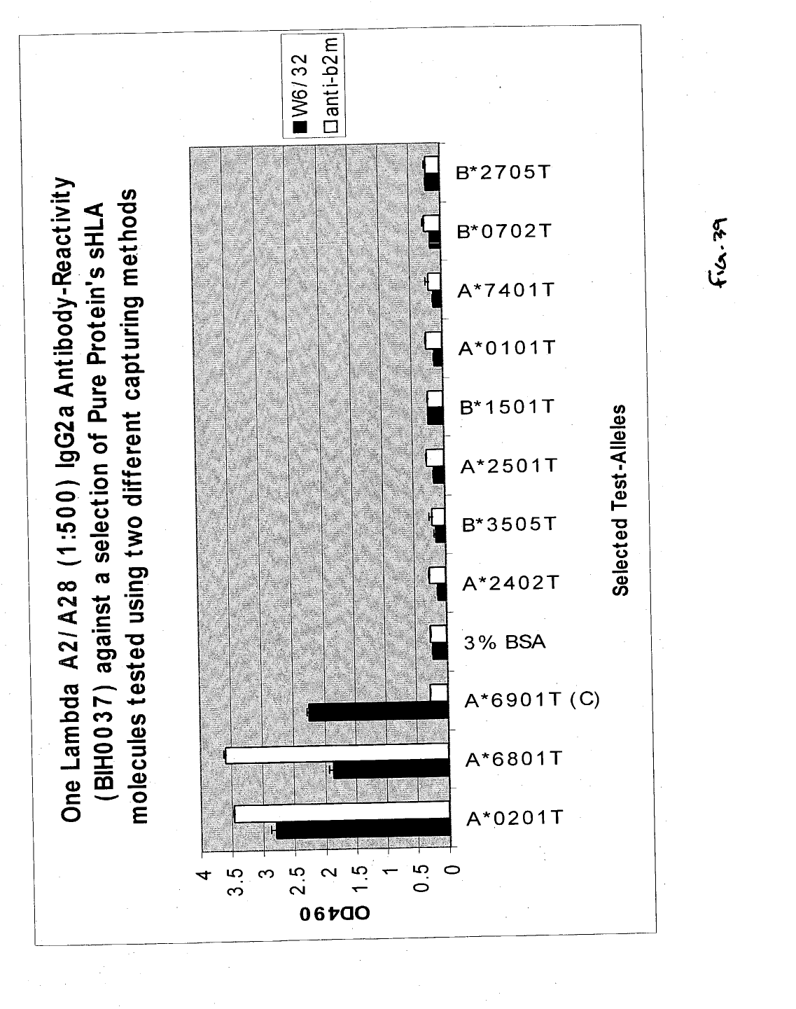

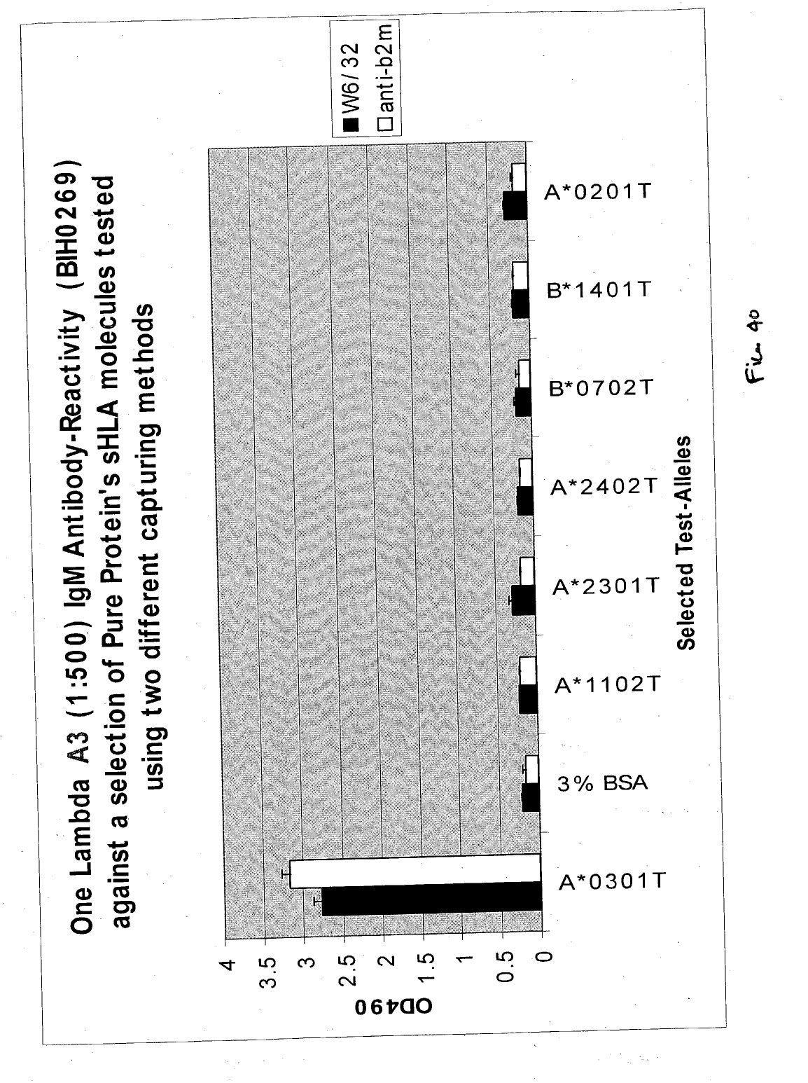

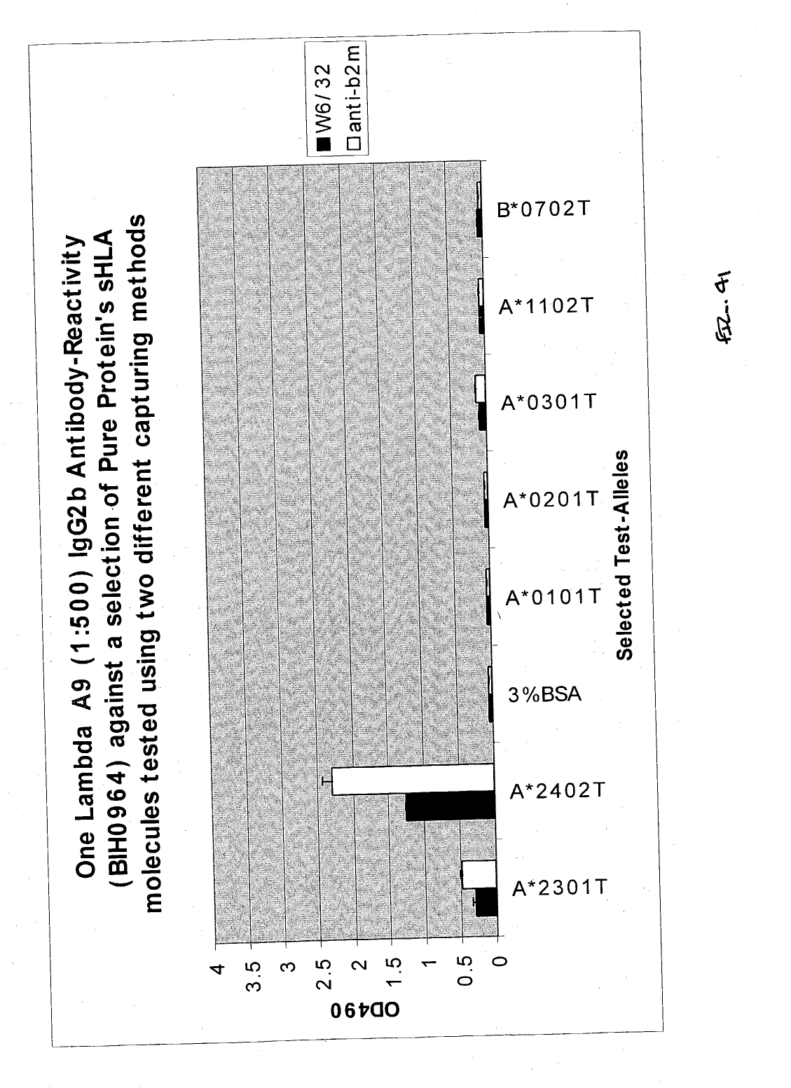

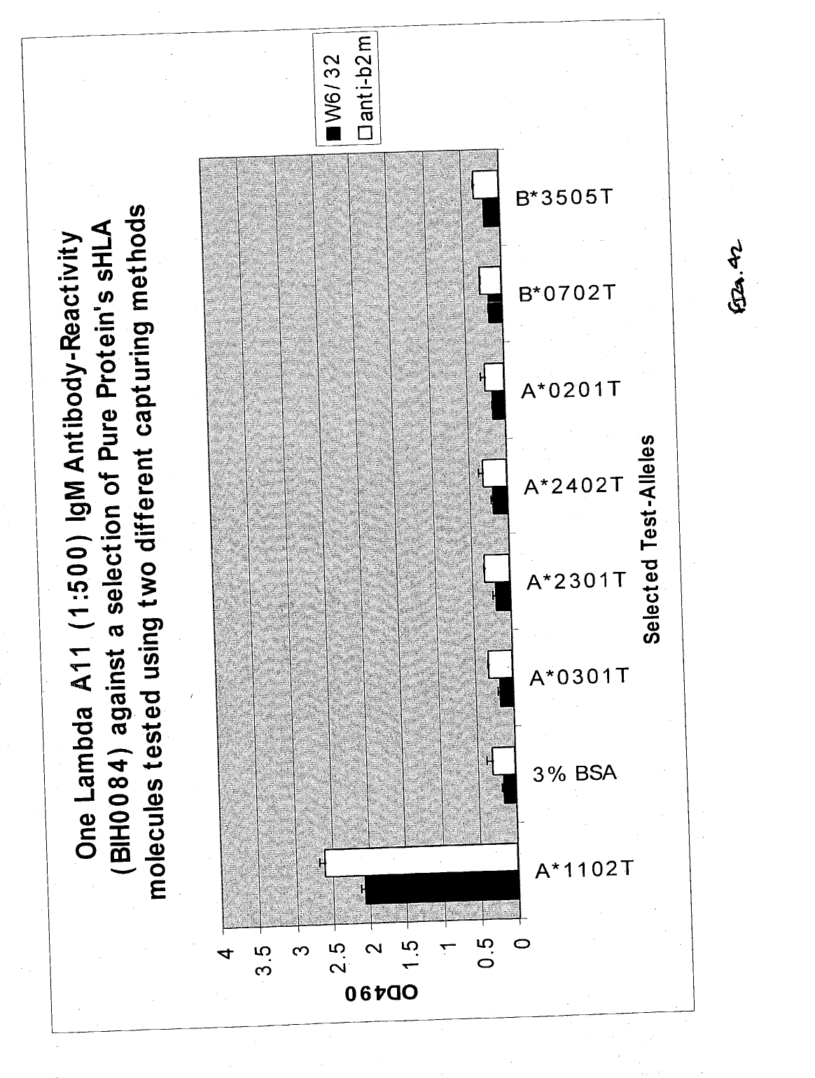

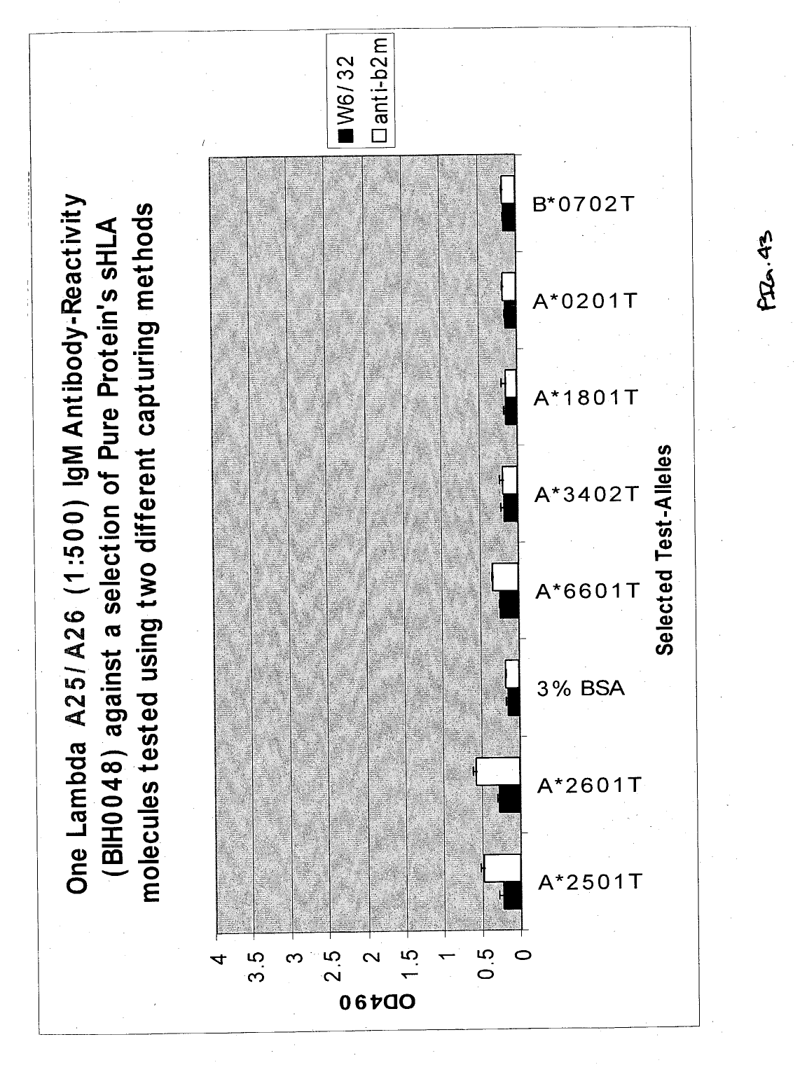

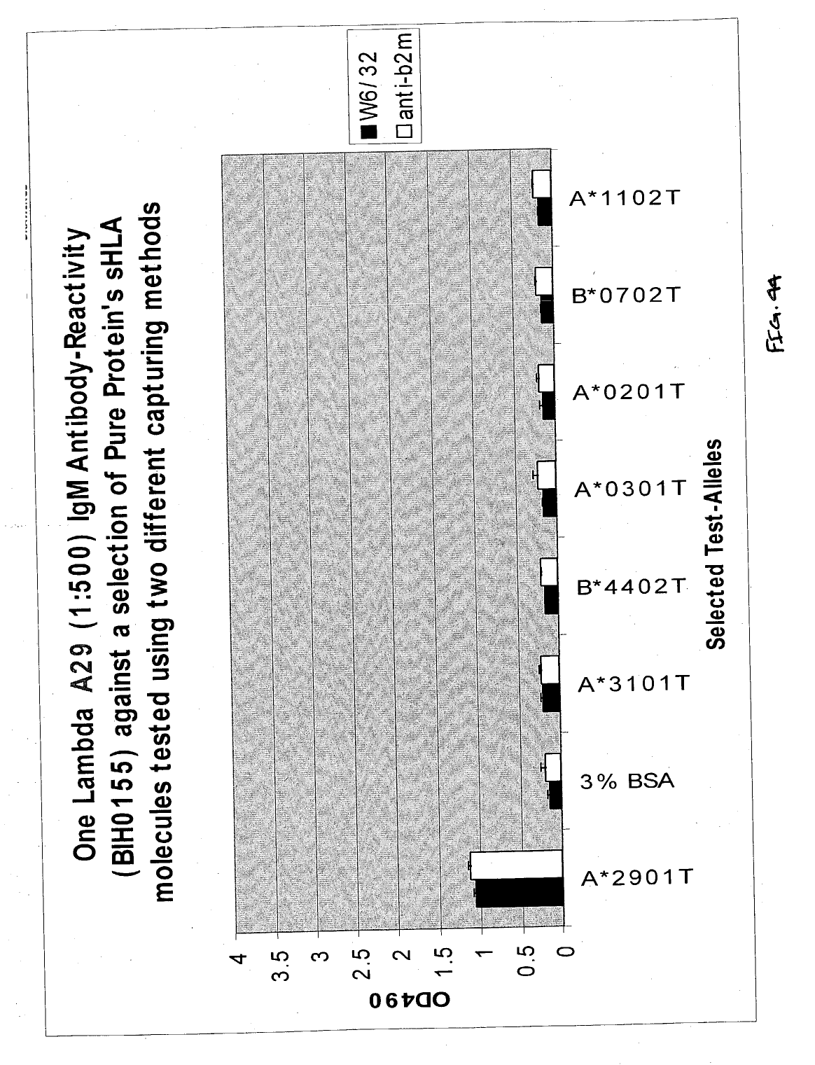

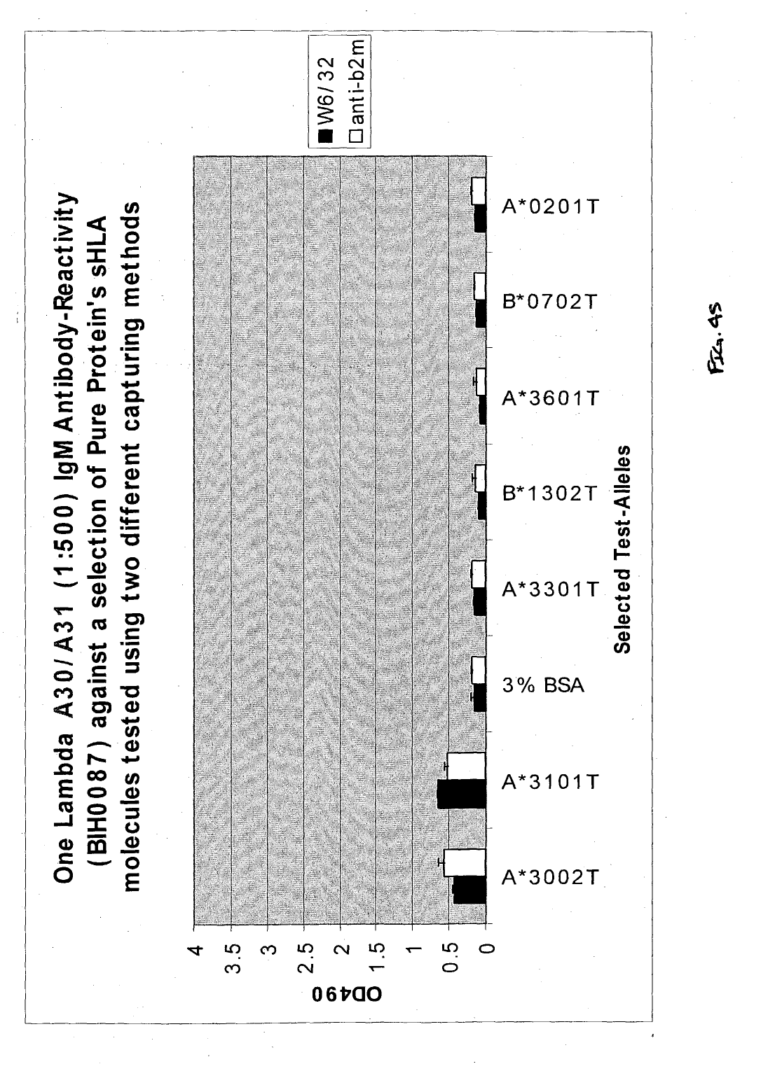

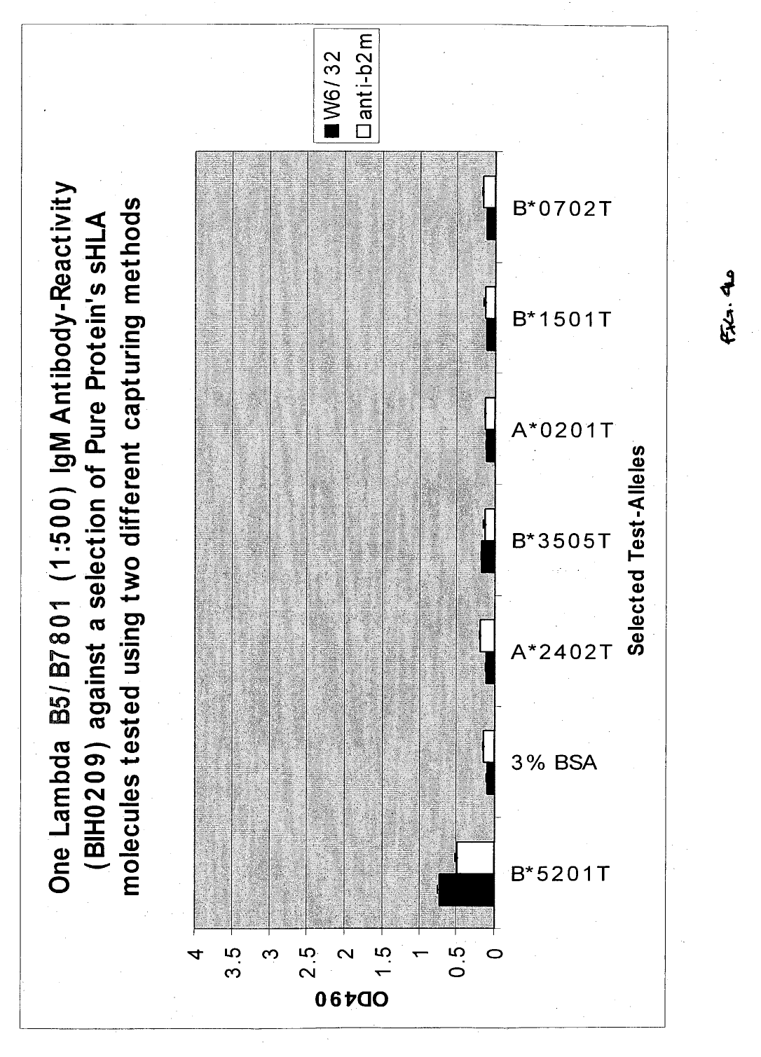

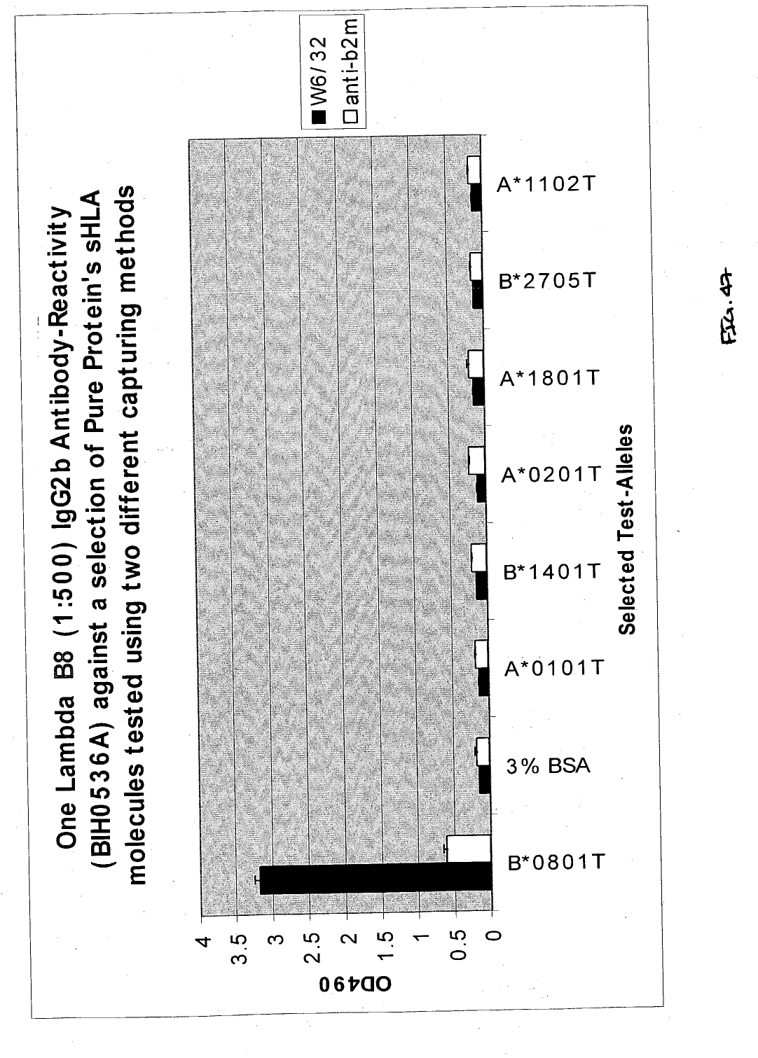

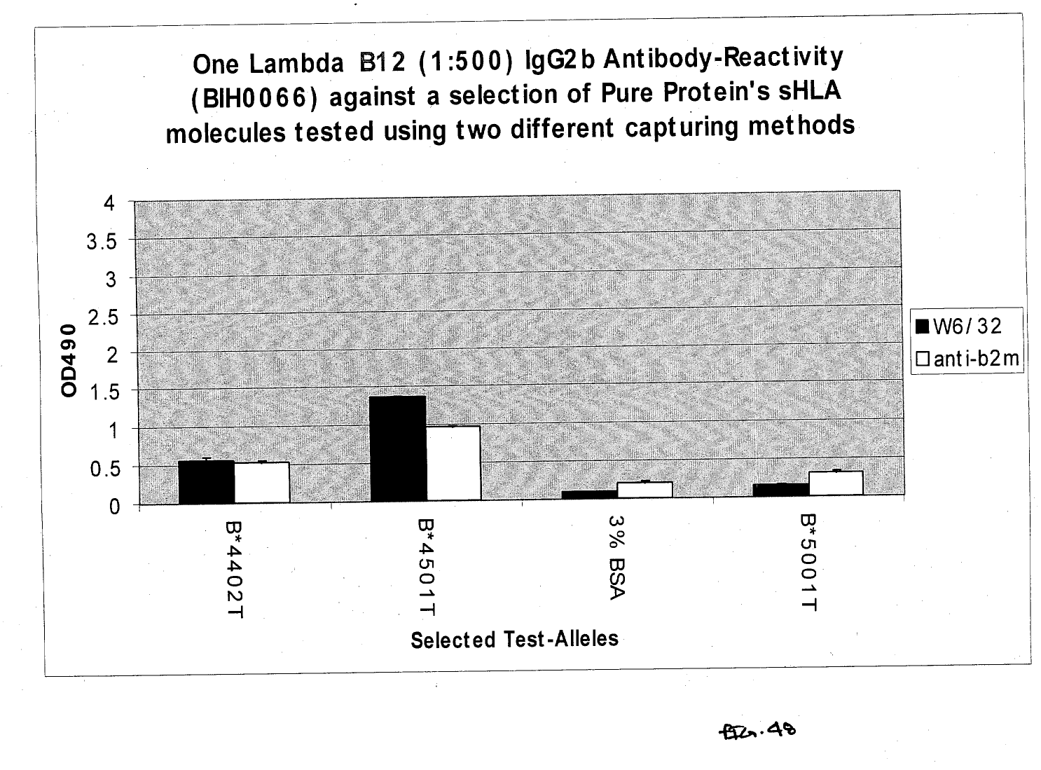

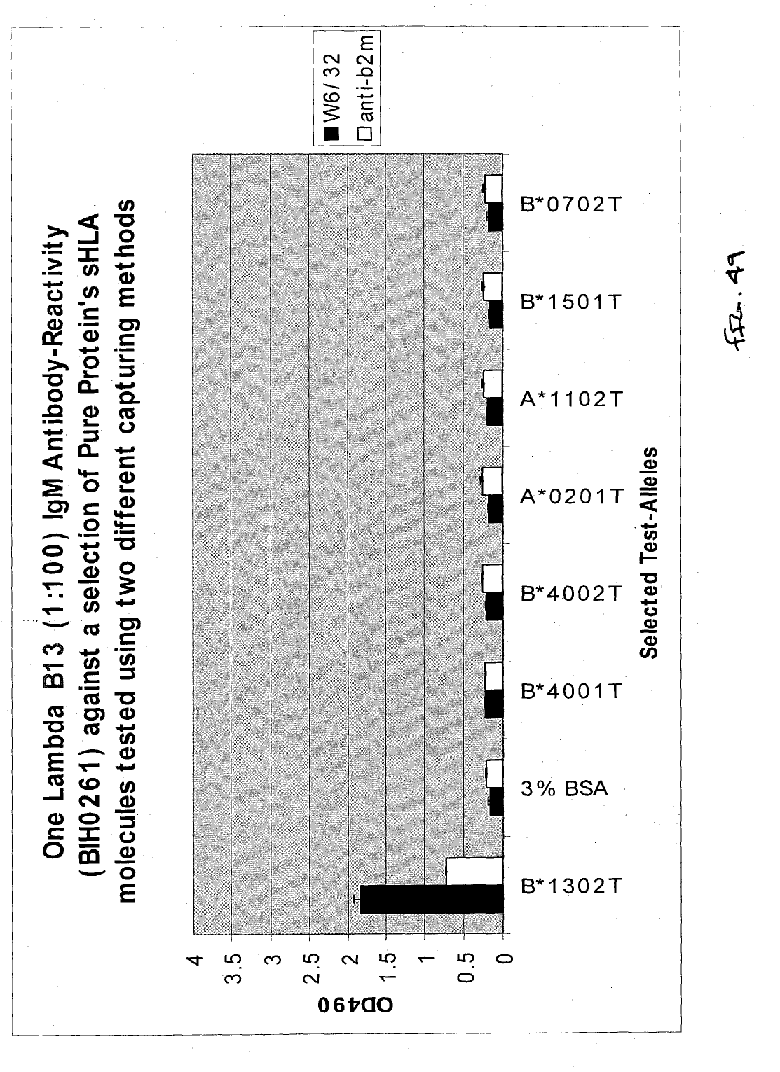

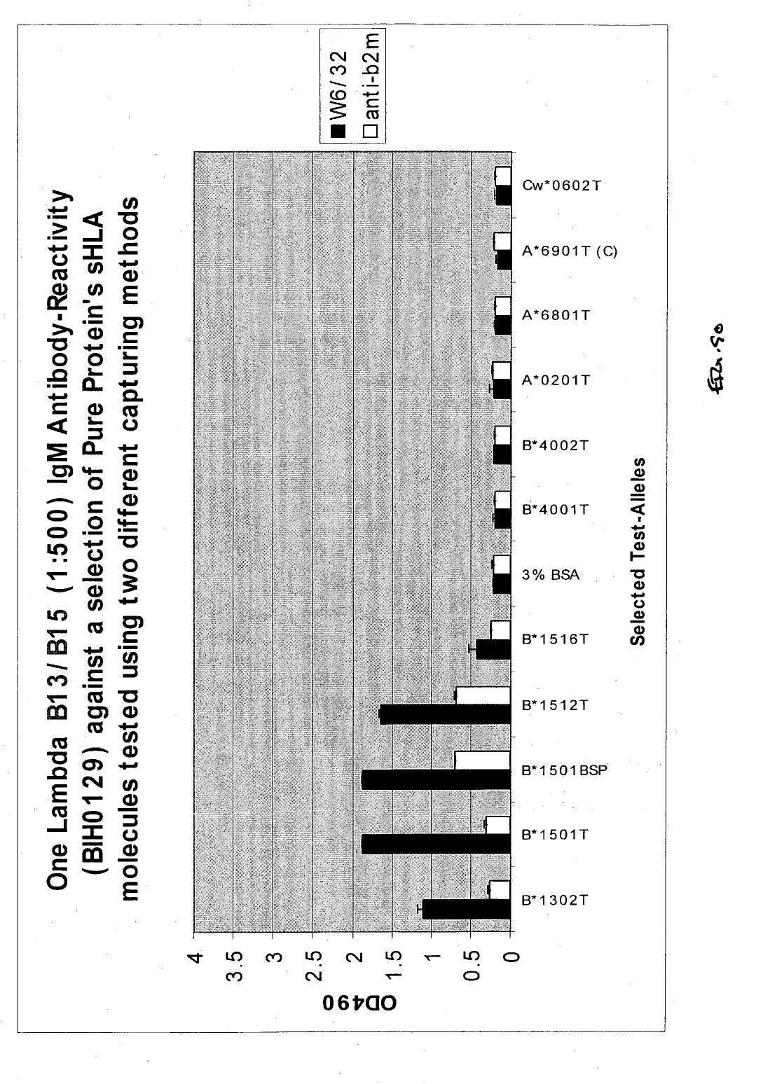

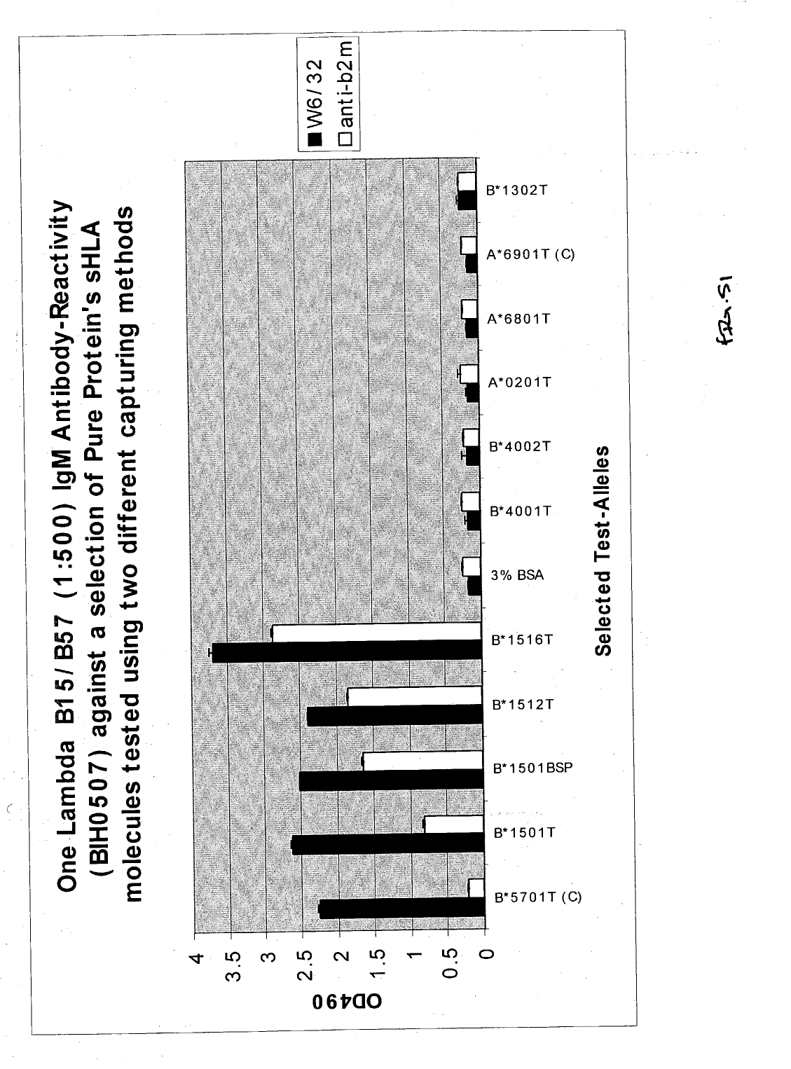

A panel of selected sHLA alleles was tested using commercially available single specificity monoclonal antibodies (FIGS. 38-51). All experiments performed resulted in the recognition of the allele corresponding to the chosen antibody. The single specificity monoclonal antibodies act as detecting antibodies. Soluble HLA is presented to the detecting antibodies through W6/32 as well as anti-β2m capturing to ELISA plates. In single cases, no purified sHLA was readily available to be tested. Thus, crude material marked with (C) was used. Because crude material does have excess amounts of free β2m which neutralize binding to anti-β2m, no signal was expected.

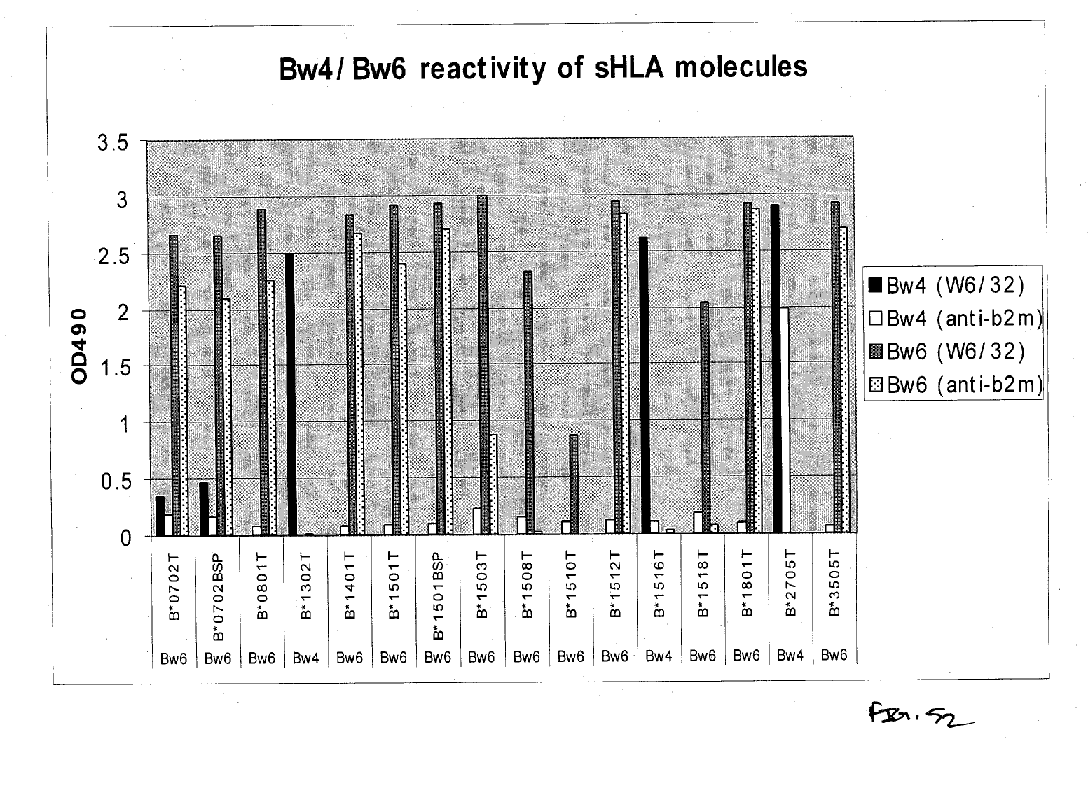

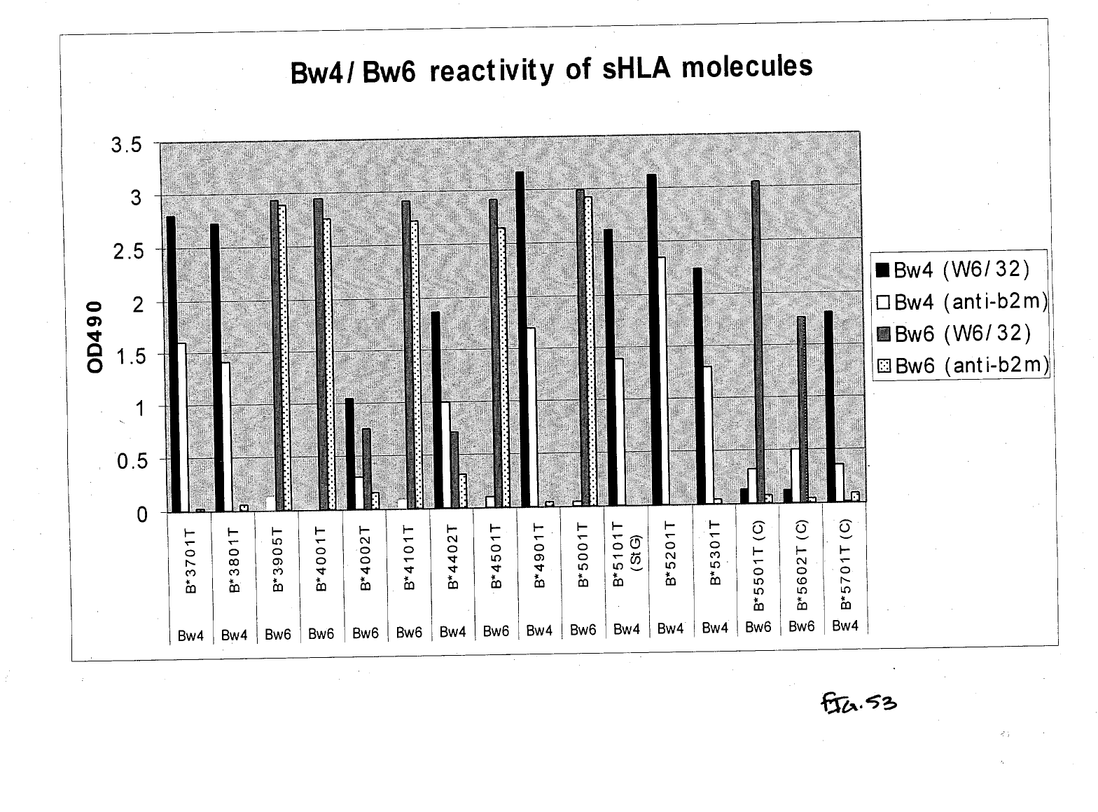

In addition, Bw6 and Bw4 Abs were tested (FIGS. 52-53). Each Ab is known to recognize a conserved epitope on B alleles. However, Bw6 positive B alleles are Bw4 negative and vice versa. These tests confirmed as expected that all purified sHLA tested harbor the Bw6 or Bw4 epitope, respectively.

5. Edman and Mass Spec Amino Acid Sequencing

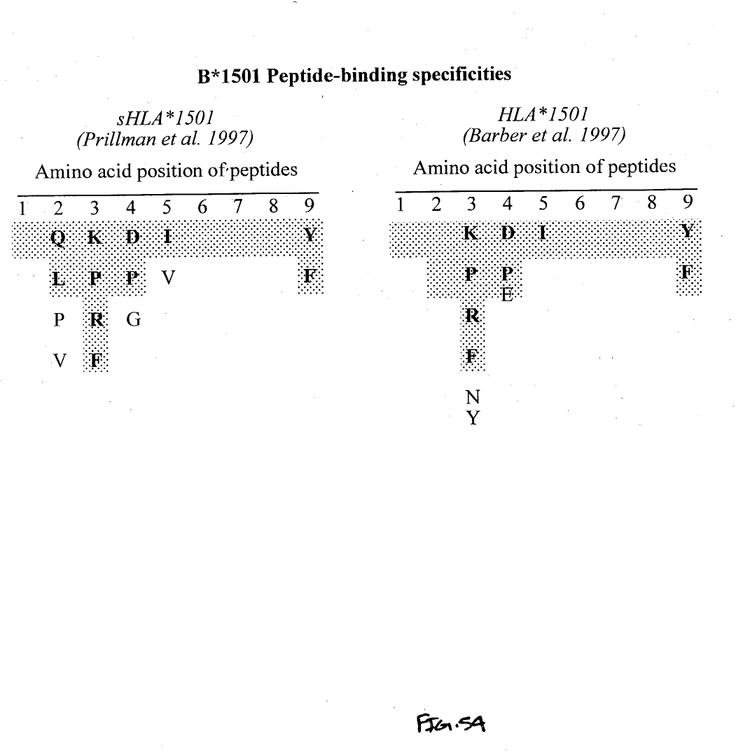

The peptides bound in the antigen binding groove of the class I molecule impact the conformation and the antibody reactivity of the class I molecule. The peptides eluted from the sHLA class I molecules produced and purified by the methods of the present invention have been characterized, and it was found that the-peptide motifs match those of membrane bound class I molecules reported by other laboratories. FIG. 54 shows a motif comparison between sHLA-B*1501 purified by the methods of the present invention and a membrane bound B*1501 motif from another laboratory. The motifs are nearly identical. The same result has been seen with six sHLA class I molecules analyzed. In addition, individual peptide ligands isolated from the sHLA purified by the methods of the present invention have been sequenced, and they match ligands found in membrane bound class I of other laboratories. Thus, the sHLA proteins of the present invention appear to traffic and bind peptides as do membrane bound class I.

6. Peptide Binding Assays

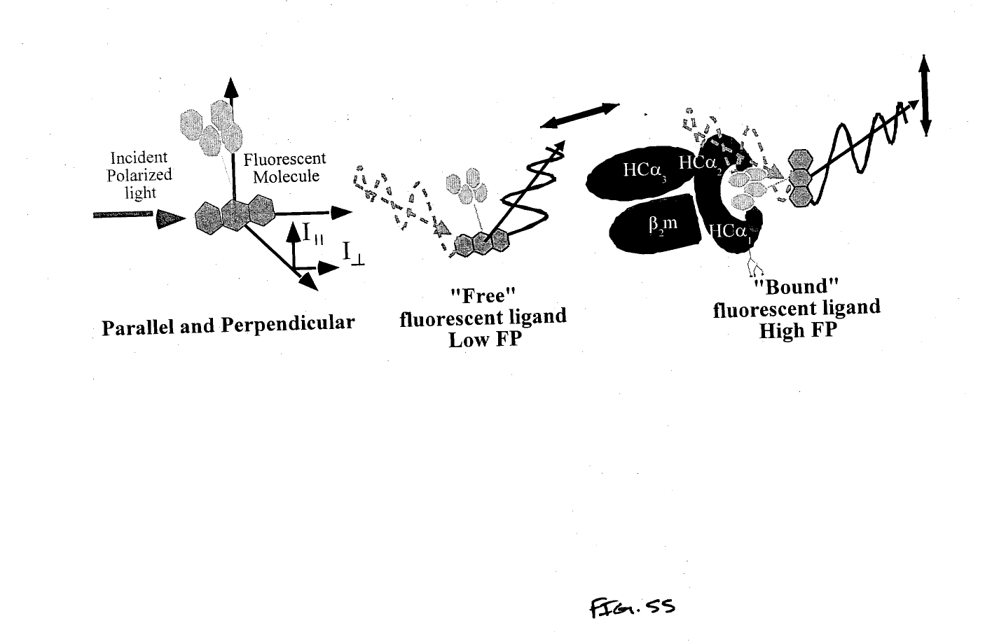

Fluorescence polarization allows the direct measurement of the ratio between free and bound labeled ligand in solution without any separation steps (FIG. 55). Ratiometric measurements are an advantage as these types of measurements can self-correct for variations caused by lamp intensity fluctuations or interferences caused by quenching of the fluorescence. In the move towards a wider adoption of fluorescence technologies, there is the added benefit of abandoning radioactive tracers, which are increasingly becoming liabilities because of their cost and safety profile. Most important, FP allows real time measurements of single reactions to determine binding kinetics as well as equilibriums. Furthermore, since no biological system can show polarization below 0 mP or greater than 500 mP, FP automatically checks assay validity. Considered a negative point in using FP is that detected values often result in the loss of about 10-90% of fluorescence intensity. This in itself may reduce the sensitivity of fluorescence polarization assay as opposed to assays with direct intensity measurements.

The technique of FP is based on the fact that if excited with plane-polarized light, the light emitted by a fluorophore is polarized as well. The angle between the planes of exciting and emitted light is highly dependent on the molecular motion of the fluorophore. FP values are defined by the equation:

$$Polarization = \frac{{I}_{∥} - {I}_{⊥}}{{I}_{∥} + {I}_{⊥}}$$

where I_{||} is the intensity of the fluorescence measured in the parallel (||) or horizontal direction (S) and I_{⊥} is the intensity of the fluorescence measured in the perpendicular (_{⊥}) or vertical direction (P).

If a fluorescent-labeled peptide binds to the sHLA molecule of higher molecular weight, the average angle (composed of the distribution of all angles between the optical planes) will decrease due to the slower molecular rotation of the bound probe (FIG. 55). Therefore, the ratio between the bound and free probe can be measured by FP directly in solution. This advantage makes FP an excellent tool for the fast and precise determination of molecular interactions between sHLA and peptide.

A binding assay was developed to demonstrate that the labeled probe will bind to the molecule of interest. The following criteria, however, must be met in order to validate the binding assay: (1) binding should be saturable, indicating a finite number of binding sites; (2) the binding should have the requisite specificity, where the binding affinity, defined as the dissociation constant (K_{d}), should be consistent with values determined for physiological molecules; and (3) ligand binding should be reversible, reflecting the dynamic nature of the chemical transmission process and reaching equilibrium when the ligand association rate is equal to the dissociation rate.

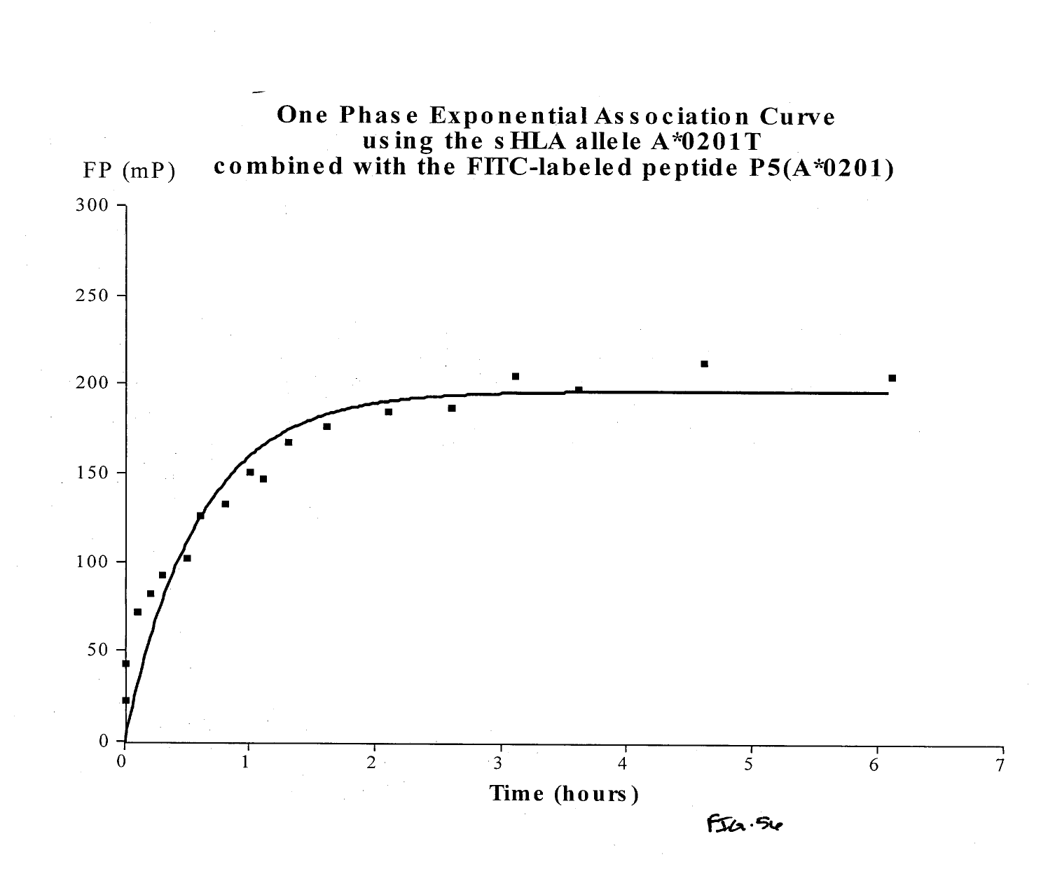

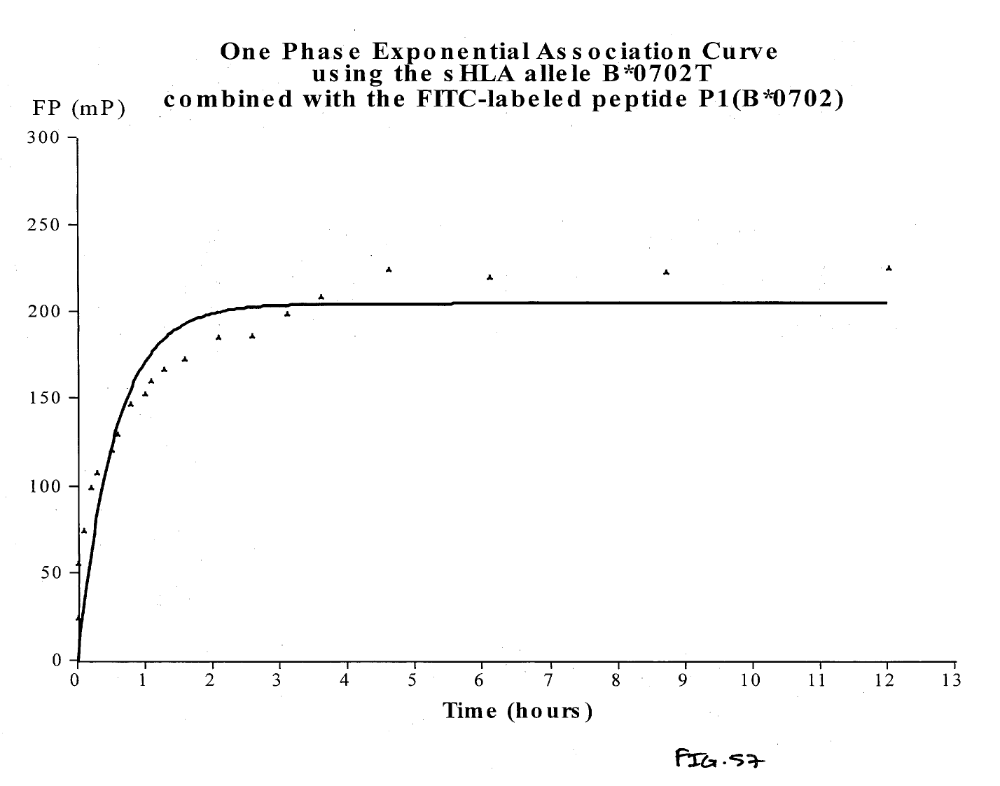

Before reaching equilibrium, the peptide follows the rules of association. In this kinetic experiment the forward (kₒₙ) rate constants of the binding process can be determined if the amount of sHLA and peptide are held constant and the time varied. Because the reaction mixture can be observed over several independent time points, each experiment's association curve is determined (FIGS. 56-57).

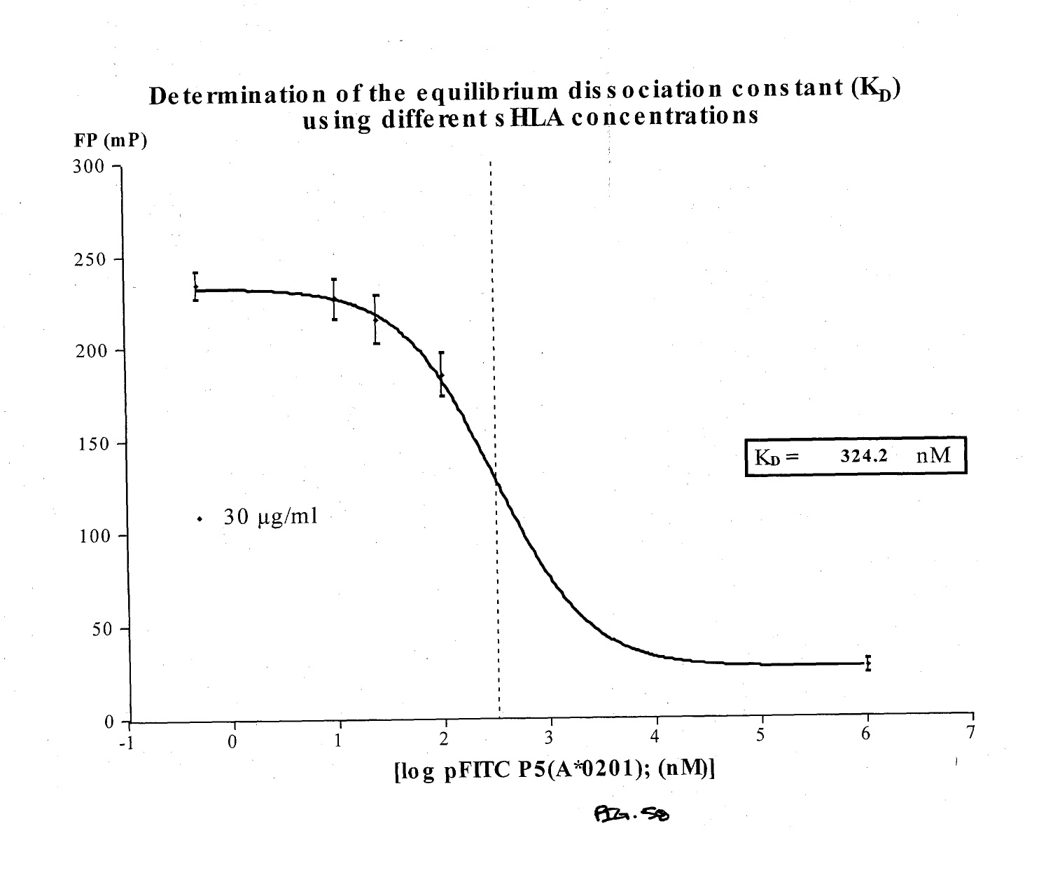

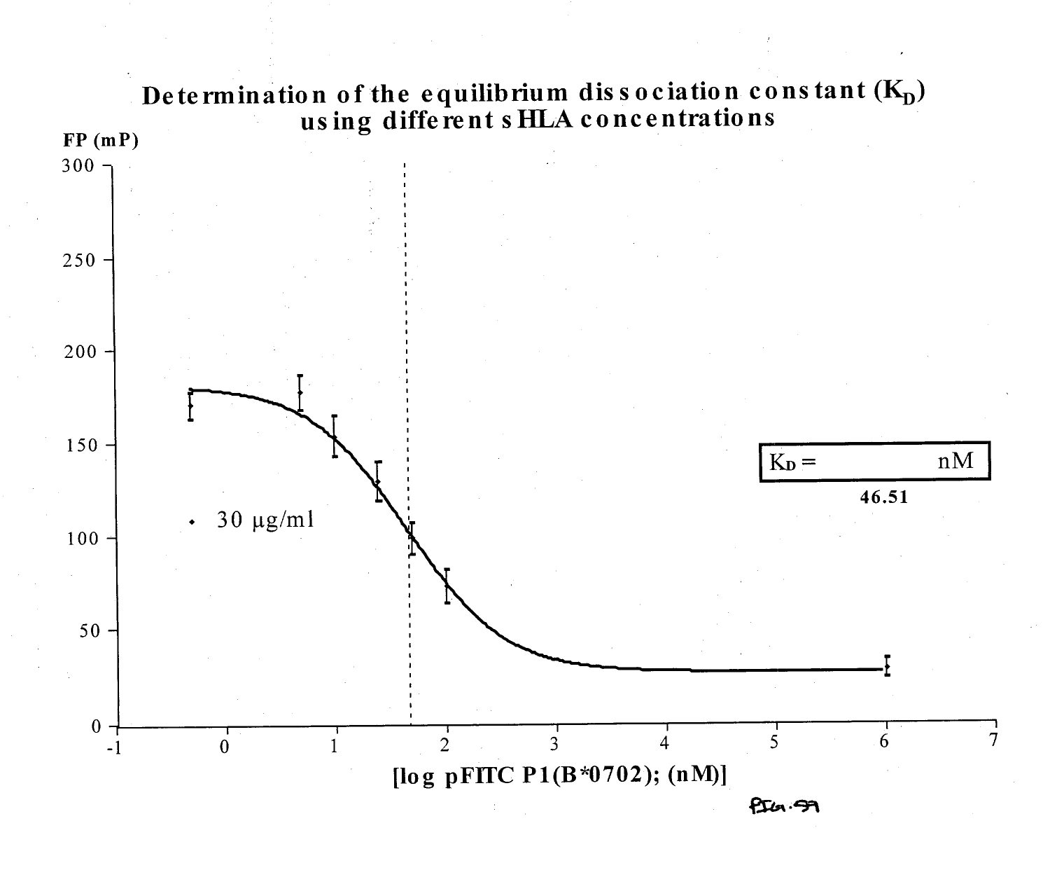

In the experimental setup shown in FIGS. 58-59, a saturation curve is generated by holding the sHLA (binder) constant. Varying the tracer concentration (dose range: 0.1 nM-1 mM) in case of constant binder (concentration of sHLA. determined above) was tested in order to determine the affinity constant (K_{d}) of the labeled peptide and to obtain a smooth saturation curve. The lower the K_{d} value, the higher the affinity of the peptide for the sHLA molecule. Only values that have reached equilibrium (Yₘₐₓ) can be used for saturation experiments.

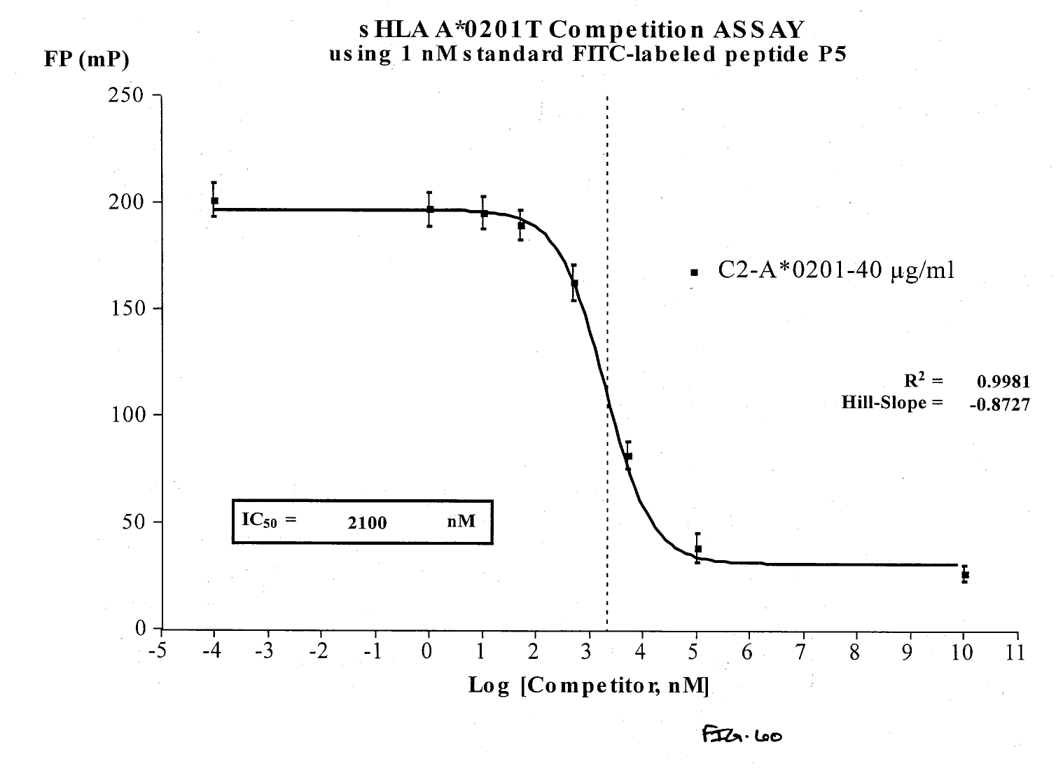

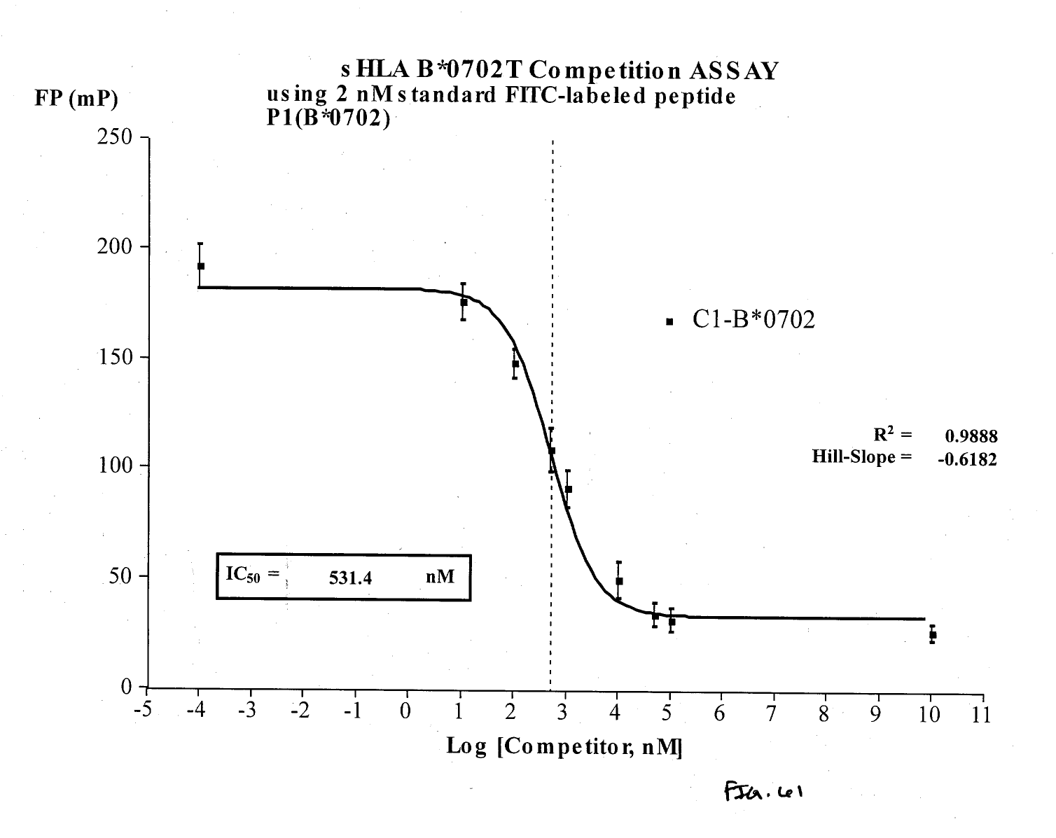

Specific binding of a fixed concentration of fluorescent-labeled synthetic peptides in the presence of various concentrations of unlabeled test competitor-peptides (dose range: 0.01 μM-100 μM) was tested (FIGS. 60-61). The concentration of unlabeled competitor peptide that produces fluorescent-labeled peptide binding half way between the upper and lower plateaus of the obtained curve will be defined as the IC₅₀. The IC₅₀ is determined by three factors: (1) the affinity of sHLA for the competitor peptide—if the affinity is high, the IC₅₀ will be low; (2) the concentration of fluorescent-labeled tracer peptide -choosing a higher concentration of tracer will take a larger concentration of unlabeled peptide to compete for half the binding sites; and (3) the affinity of tracer peptide for sHLA (K_{d}). It takes more unlabeled competitor peptide to compete for a tightly bound tracer peptide (low K_{d}) than for a loosely bound tracer peptide (high K_{d}). To achieve the highest sensitivity and accuracy of the competition assay, the parameters identified will be optimized to the point where the lowest concentration of a competitor test peptide results in a clearly distinguishable, positive response. No competition should be detected in the case of using an irrelevant unlabeled competitor peptide.

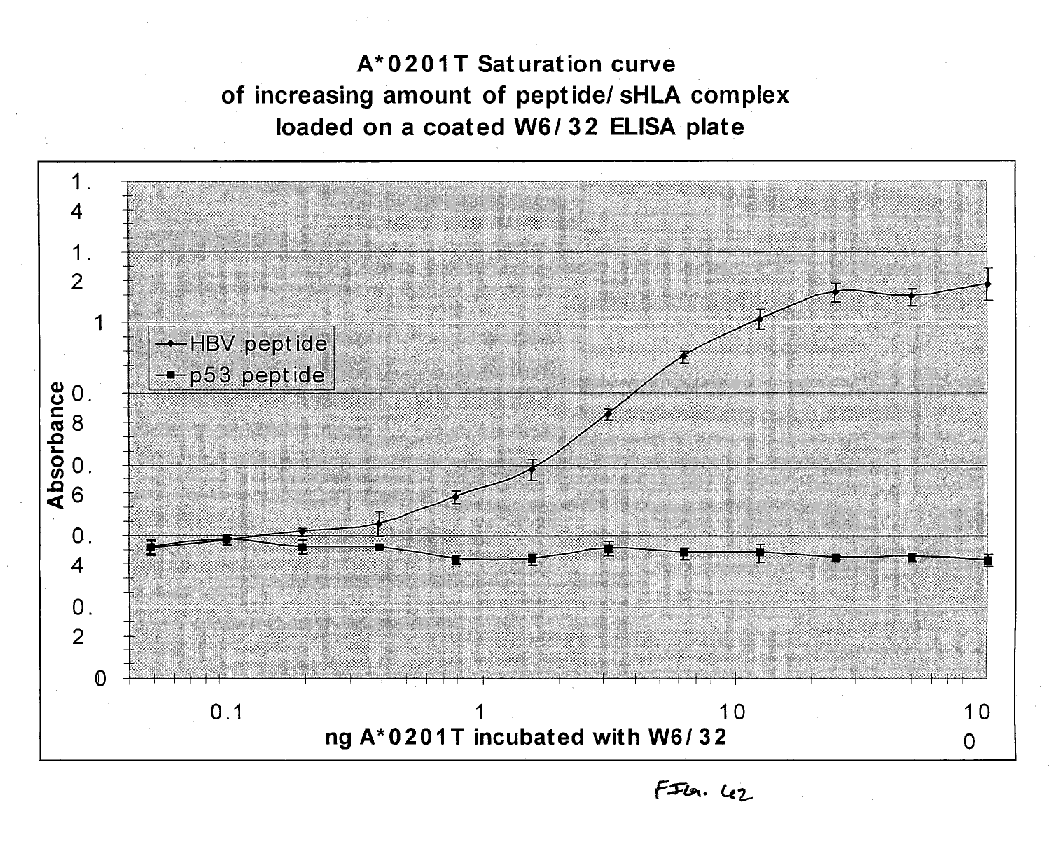

As seen in FIG. 62, an HBV peptide known to bind strongly to A*0201T was used to replace the endogenous peptide in solution. After incubation for 48 hours at room temperature, the sHLA complexes were immobilized on a solid support through the HLA specific antibody W6/32. The HBV peptide/A*0201T complex was then detected using a highly specific antibody only recognizing this particular conformation. Saturation of the W6/32 coated ELISA plate could be achieved, demonstrating the binding of the HBV peptide to sHLA molecules and a successful replacement of the endogenous peptides with the HBV peptide. No saturation was detected using the irrelevant peptide p53, indicating that peptide p53 as well as endogenous peptides do not contribute to the specific signal obtained by the HBV peptide/A*0201T complex selective antibody.

Storage and Handling of Individual, Soluble MHC Molecules

Each protein may have specific requirements once it is extracted from its normal biological milieu. If these requirements are not satisfied, the protein can rapidly lose its ability to carry out specific functions, and an already limited lifetime may be drastically reduced. Thus, failure to determine and manage these requirements has often been a major hurdle in obtaining successful protein characterization. In some cases, the difficulty has been to stabilize the protein against external proteolysis, while in other cases the problem has been to maintain ligand-binding or enzymatic activity. Solutions to these problems are highly specific.

A buffer is defined as a mixture of an acid and its conjugate base which can reduce changes in solution pH when acid or alkali are added. The selection of an appropriate buffer is important in order to maintain a protein at the desired pH and to ensure reproducible results. Buffers are often present at the highest concentration of all components in a protein solution and may have significant effects on a protein or enzyme.

The experimental approach described herein shows that various buffers are suitable for use herein. PBS, pH 7.4, was chosen as standard buffer since it is creates a stable surrounding and does not have supplements that could possibly interfere with downstream applications. Phosphate-buffered solutions are highly susceptible to microbial contamination. To prevent buffer contamination during storage, 0.02% (3 mM) sodium azide was used. Sodium azide does not interact significantly with proteins at this concentration. Refrigeration helps to reduce buffer contamination.

Very dilute protein solutions are highly prone to inactivation and often lose activity quickly, possibly via denaturation at surfaces such as glass and plasticware. This is especially true of oligomeric proteins where dissociation of subunits can occur at low concentrations. The individual polypeptide chains comprising the oligomer may denature. High protein concentrations (>2 mg/ml) provide some auto-buffering capacity. Thus, protein solutions of concentration <1-2 mg/ml are concentrated as rapidly as possible in the procedure described herein.

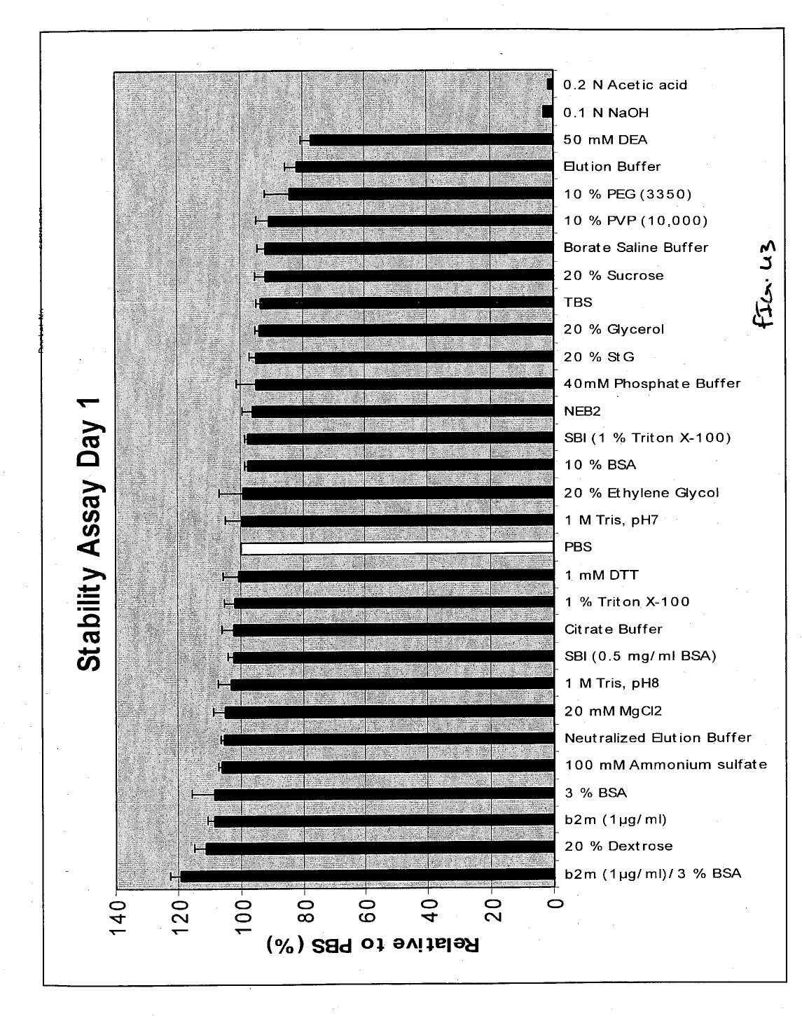

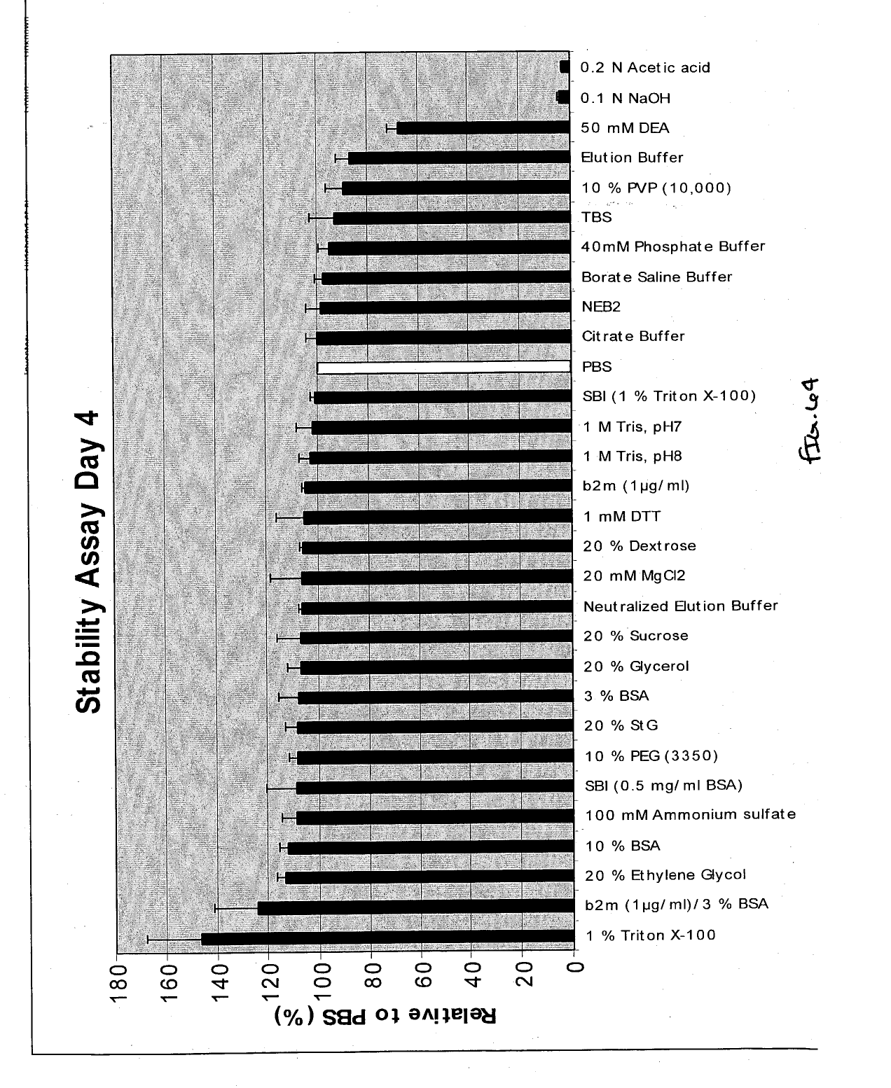

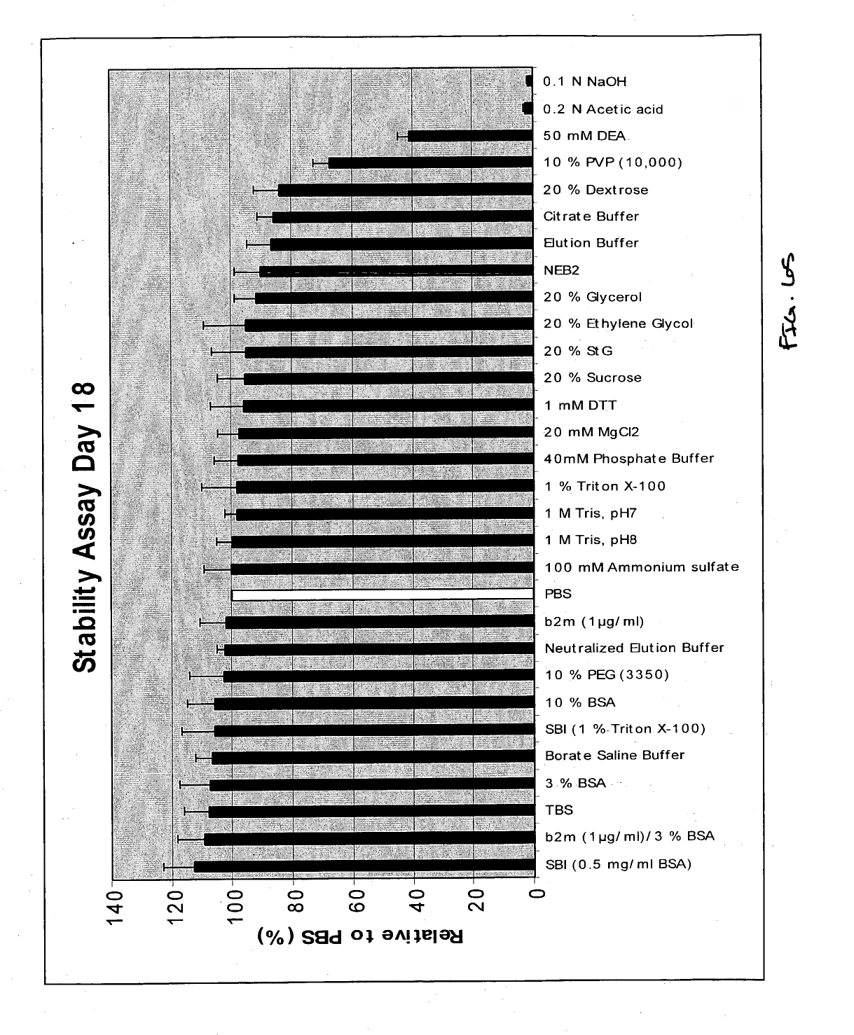

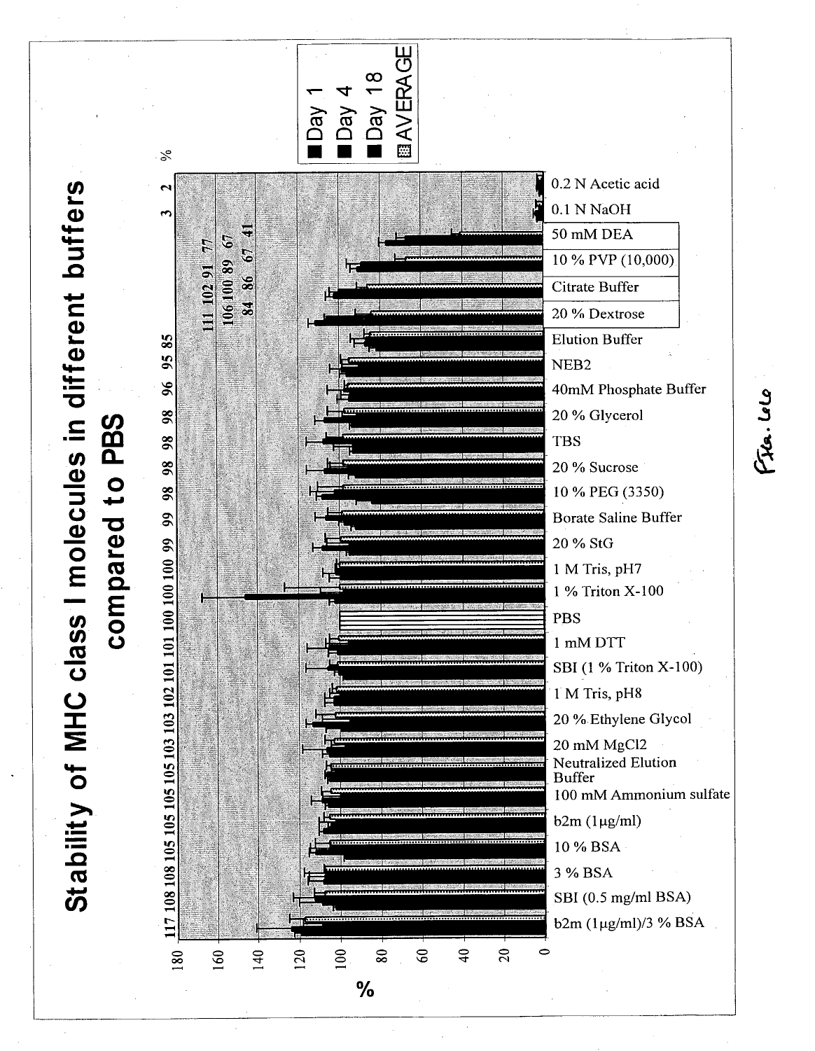

In the stability assay shown in FIGS. 63-66, sHLA B*1512T was incubated in different buffers and solutions at a concentration of 55 μg/ml over a time period of 1, 4, and 18 days at 4° C. After the incubation time, an ELISA was performed, using W6/32 as the capture antibody and anti-β2m(HRP) as the detector antibody. The ELISA results were standardized using PBS as 100%.

This experiment clearly demonstrates a high stability of sHLA over a wide range of buffers and solutions. Only 0.1 N NaOH and 0.2 N acetic acid were able to completely abolish the reactivity of the molecule.

The stability in elution buffer is only 85% compared to PBS, justifying an immediate buffer exchange during the purification procedure. Only four solutions, 20% Dextrose, Citrate buffer, 10% PVP and 50 nM DEA were found to show declining stability over time, whereas the others seem to be constant over the time period tested.

The value of Triton X-100 at four days appears to be the highest value achieved during the whole assay. However, it also shows a high standard deviation value. It appears to be more likely to be an outsider result due to a dilution mistake rather than increased stability of sHLA after 4 days. This value was not considered in calculating the average.

Generally, PBS seems to be an optimal storage and reaction buffer. Only buffers containing BSA seem to perform slightly better than PBS alone. Choosing 3% BSA in our ELISA seemed to be a good choice, confirmed by the above results.

Kinetic stability is usually measured at elevated temperatures, but the inactivating event(s) at high temperatures may not mirror those at the much lower temperatures used for storage. It is not feasible, however, to monitor stability in. real time at the actual storage temperature. Fortunately, there is a methodology that can in many cases overcome these difficulties, namely accelerated degradation testing. This involves the periodic assay of samples incubated at different temperatures and use of the Arrhenius equation to predict shelf lives at temperatures of interest.
Ink=−Eₐ/RT
where k is the first-order rate constant of activity decay, Eₐ is the activation energy, R is the gas constant, and T is the temperature in Kelvin. This log form of the Arrhenius equation yields a straight-line plot of Ink against 1/T with slope −Eₐ/R. Extrapolation of this plot can give the rate constant (and hence the useful life) at a particular temperature. Accelerated storage testing has been used as a practical means of quality assurance for biological standards (Jerne, N. K. and Perry, W. L. M. (1956) The stability of biological standards. Bull. Wld. Hlth. Org. 14, 167-182, the contents of which are hereby expressly incorporated herein in their entirety.).

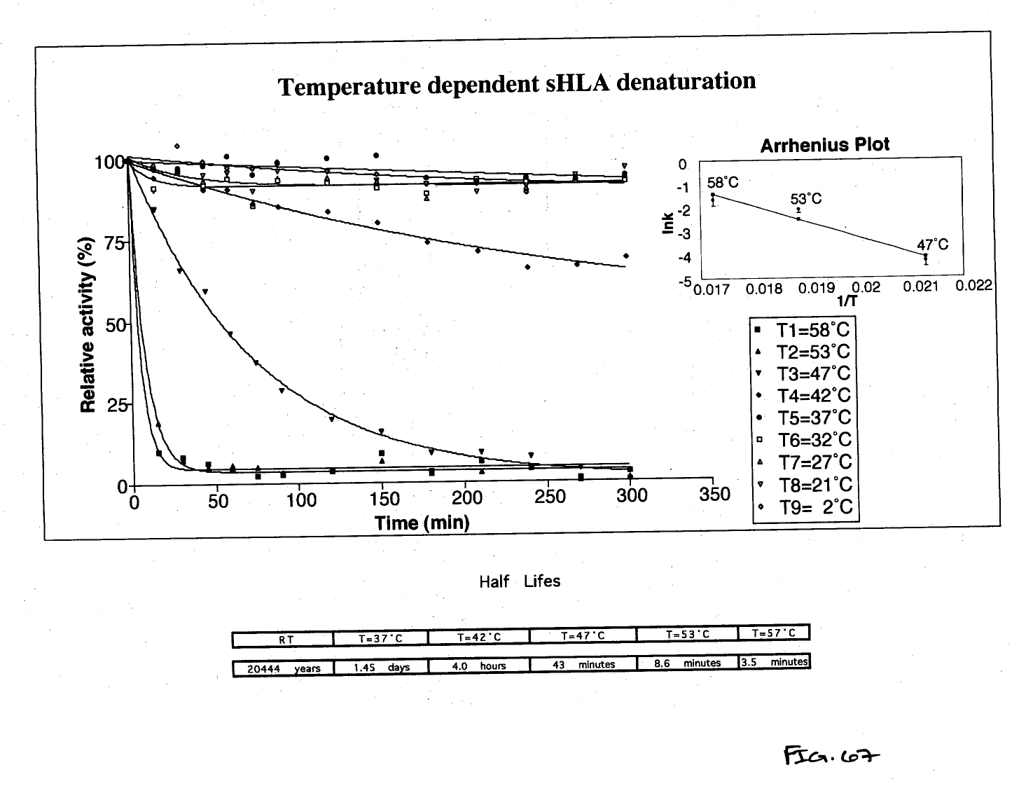

Maintaining the stability of the purified sHLA complex by identifying optimal storage and handling parameters was one of the main interests of the present invention. It has been determined through the above studies that PBS and concentrations of sHLA above 2 mg/ml are advantageous to maintaining stability. In the following experiment, the influence of temperature to the sHLA complex was tested to determine the half-life of the purified product (FIG. 67). As in the above studies, the standard sandwich ELISA procedure (W6/32/sHLA/anti-β2m-HRP) was used to measure sHLA activity in solution. Identical samples of sHLA molecules were incubated at various temperatures over a time period of 300 minutes. After heat incubation, the samples were immediately cooled to 4° C. and assayed to determine the percentage of lost activity relative to non-heated samples (stored at 4° C.) tested at equal time points. The results show a rapid loss of activity when heated above 53° C. This can be interpreted as dissociation of intact sHLA molecules. The more energy that was applied, the faster was their dissociation rate. Below temperatures of 32° C., sHLA molecules seem to be very stable. Using an Arrhenius plot, half lives for T=57° C. (3.5 min); T=53° C. (8.6 min); and T=47° C. (43 min) were calculated. Extrapolation of the graph to room temperature resulted in a calculated half live of more than 20,000 years. These results indicate that sHLA molecules are highly stable and will maintain their structural integrity if stored properly. The quality seems to be more than appropriate for commercial and other experimental purposes.

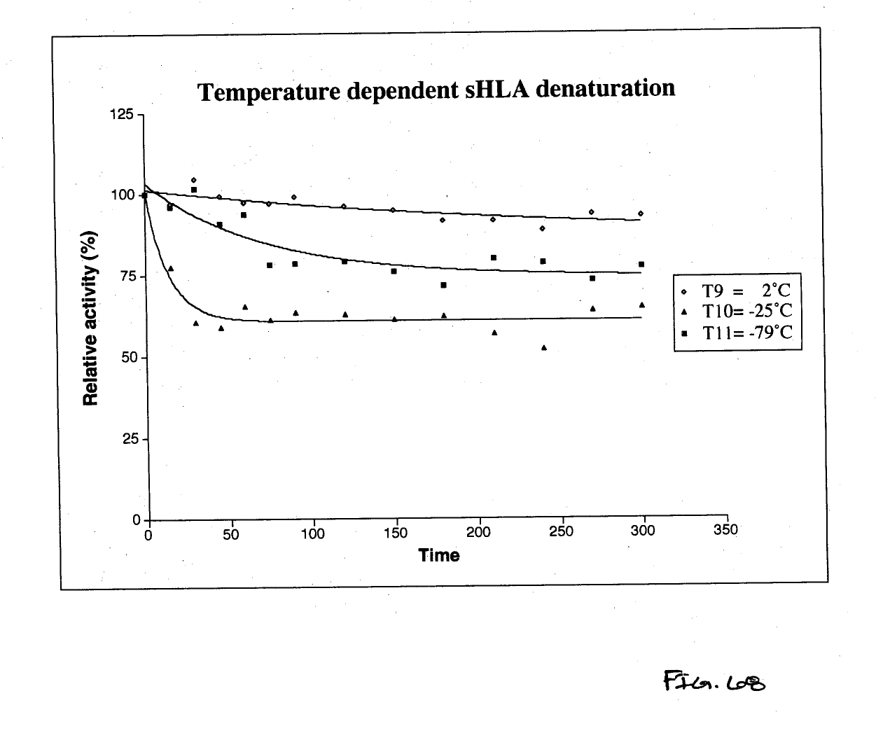

A single freeze-thaw cycle at −20° C. or −80° C. does reduce activity and is therefore not recommended (FIG. 68). A storage temperature of 4° C. is optimal. It is known that loss of purified protein due to nonspecific adhesion onto glass surfaces (1 μg of protein is absorbed on 5 cm² of a glass surface) has to be expected and will significantly diminish the amount of protein in a reaction. To probe for nonspecific adhesion, a tube test was developed to examine several different storage vessels. To overcome this problem, a variety of potential blocking agents were tested.

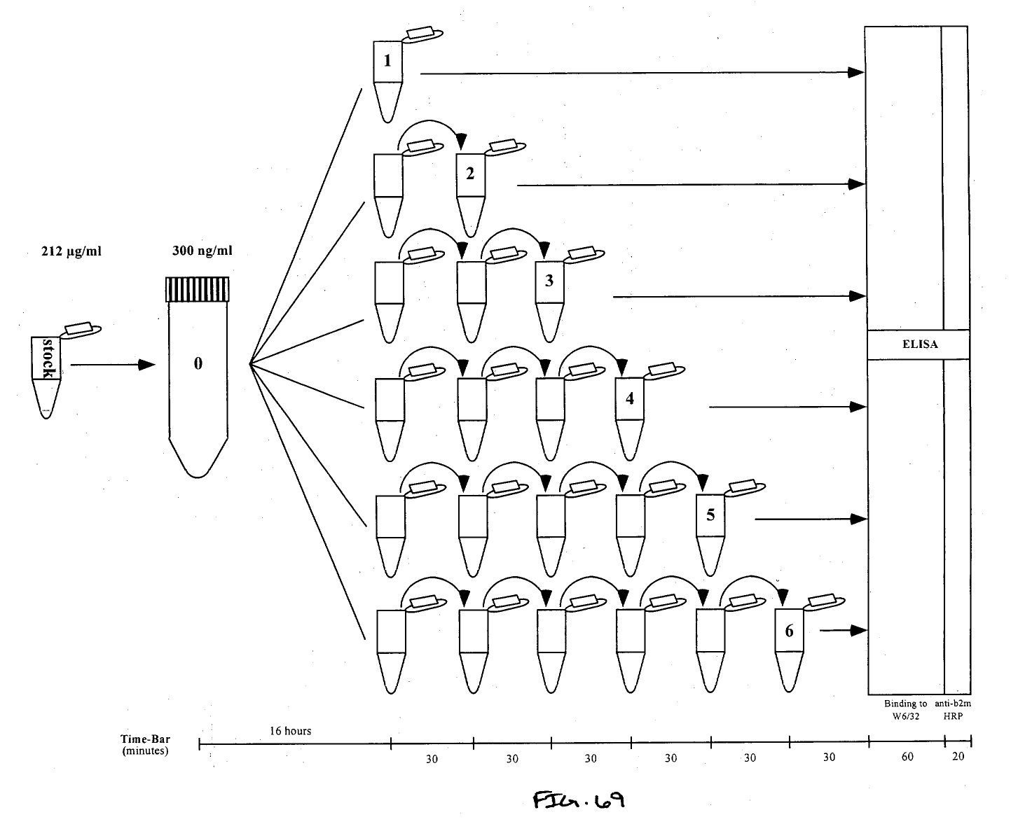

FIG. 69 demonstrates the experimental procedure. From a protein stock, a dilution of 300 ng/ml was mixed in PBS. To equilibrate the diluted sample, it was mixed 16 hours before starting the experiment and stored at 4° C . After this time, liquid was removed from one tube to another every 30 minutes. If sHLA adheres to the tube, a step-wise reduction in concentration from tube 1 to tube 6 should be observed. Successful blockers should prevent loss of protein and the step-wise reduction in concentration should not be observed or be highly diminished.

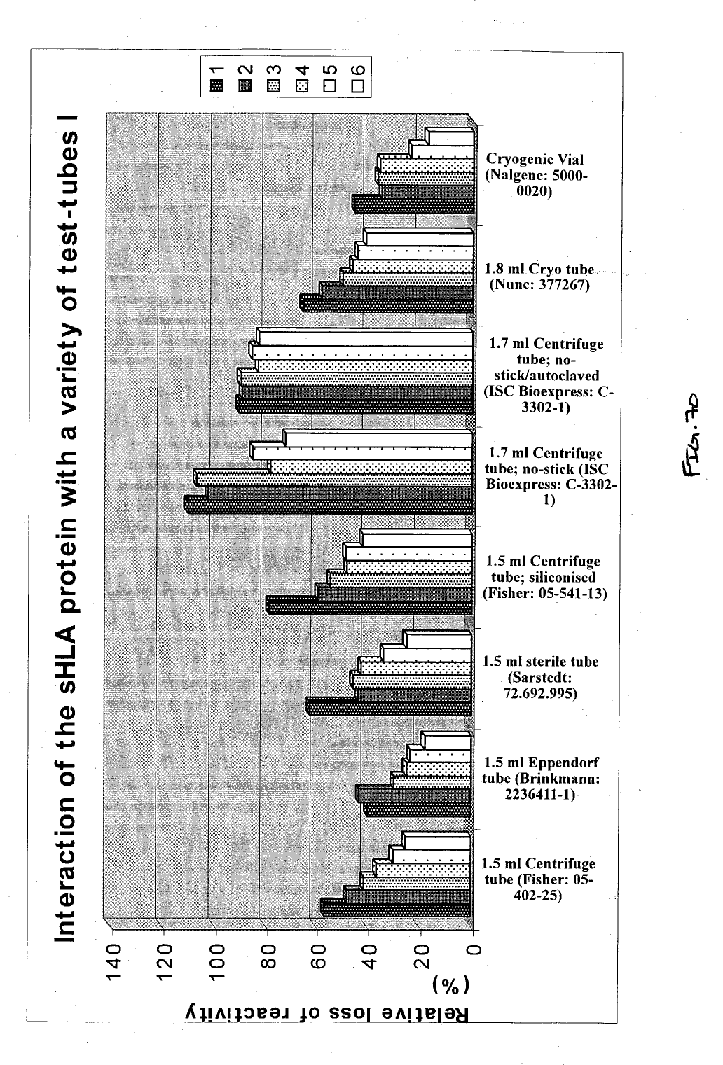

Addition of a standard sample (tube 0) to a variety of different microcentrifuge tubes or cryo vials showed profound effects on the reactivity of sHLA (FIG. 70). One of the most used 1.5 ml tubes from Fisher (05-402-25) showed a step-wise reduction in concentration from tube 1 to tube 6 as expected for vessels binding protein, losing up to 40% of reactivity during the first transfer. The same effect was seen with several other microcentrifuge tubes having adhesive potential for sHLA, and some of them showed more or less binding. The best performer was the “No stick” hydrophobic RNase/DNase free microcentrifuge tubes (Gene Mate-ISC Bioexpress, Kaysville, Utah). However, autoclaving did partially destroy these properties. These “No stick” hydrophobic tubes are specially treated with a proprietary non-reactive lubricant to have an extremely hydrophobic surface (e.g., Teflon). Siliconized tubes performed better in conserving the molecules reactivity than normal uncoated polystyrene tubes.

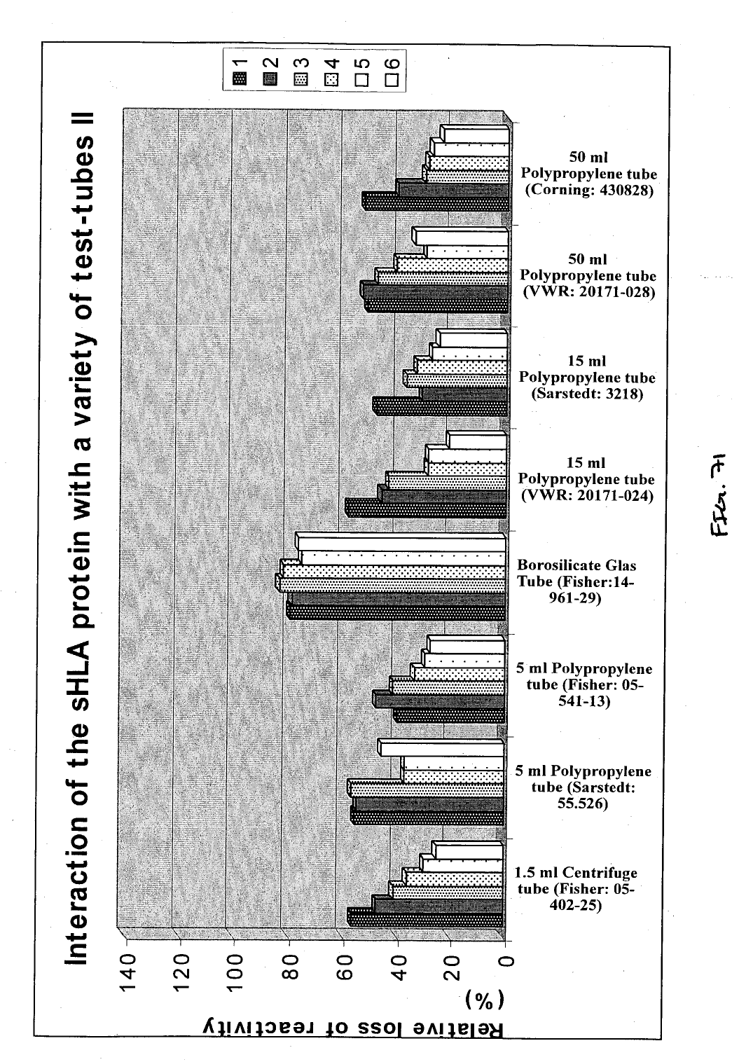

Tubes with larger volume capacity performed no better than the Fisher microcentrifuge tube (FIG. 71). Here, an exception was borosilicated glass tubes, which did not bind protein and only caused a loss of reactivity of 20%. To solve the problem of loss of reactivity, the tubes need either to be coated with a blocking agent or the blocker should be added directly to any molecule dilution. Dilute protein solutions are highly prone to inactivation and lose activity quickly, possibly via denaturation at surfaces such as glass and plasticware. High protein concentrations provide some auto-buffering capacity. Where the usage of high concentrations is not possible, inactivation may be prevented by addition of an exogenous compound.

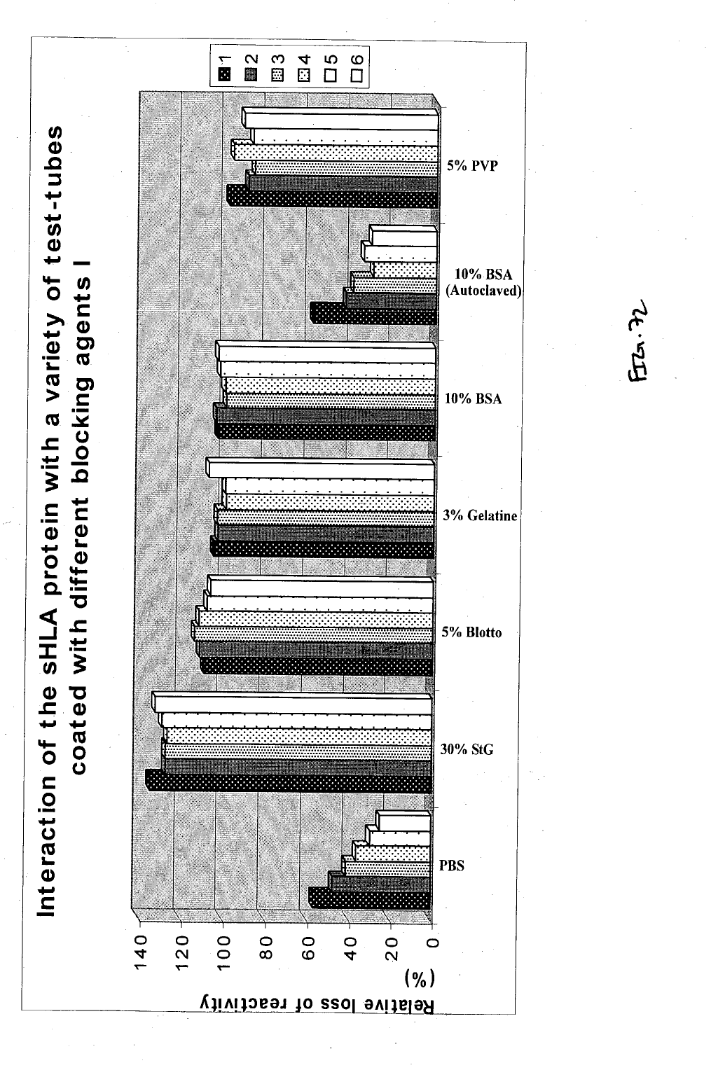

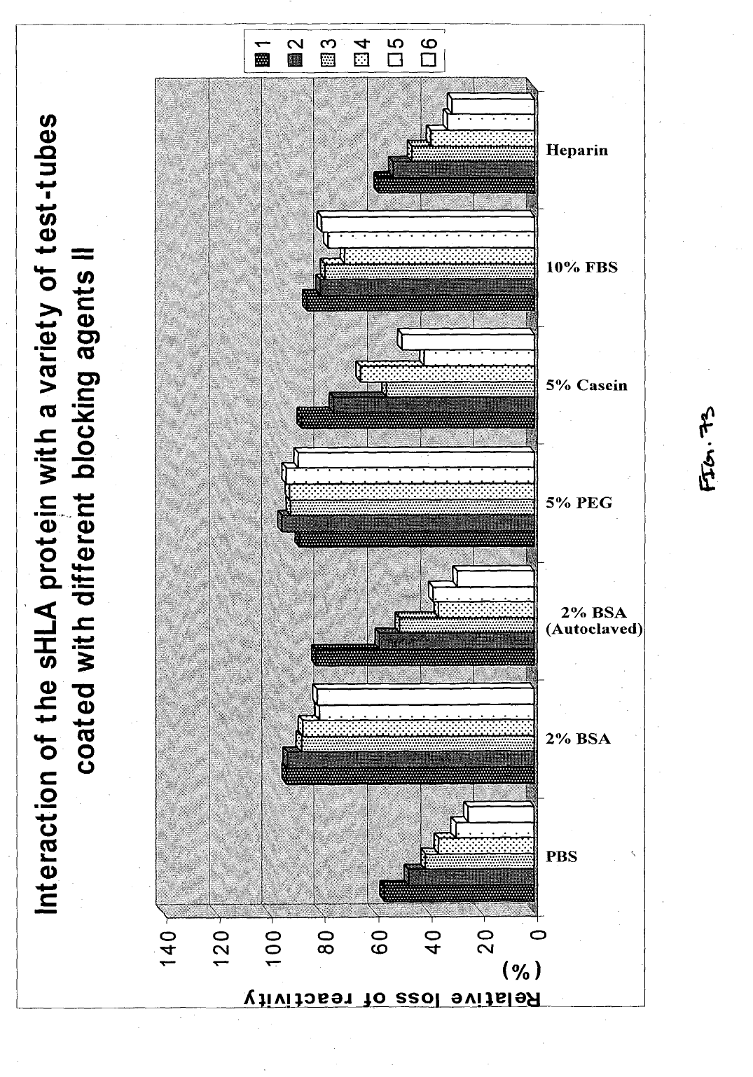

Blocking agents used to coat Fisher (05-402-25) microcentrifuge tubes were tested for their ability to prevent inactivation and/or adhesion to the surface (FIGS. 72-73). The tubes were incubated with the blocker overnight at 4° C., extensively washed with PBS and finally air dried to remove any traces of liquid. 10% BSA, 3% gelatine or 5% Blotto (milk) worked best and did not result in any loss of protein or activity compared to the tube preincubated with PBS. Usage of StabilGuard Biomolecule Stabilizer (Surmodics, Eden Prairie, Minn.; SG01-0125) coated to the tube walls highly protected the protein against tube surfaces. However, the ELISA resulted in higher concentrations than actually put into the tube. A problem using this blocking solution is its unknown composition (the manufacturer was not willing to reveal all components, but low molecular weight PVP is one of its components). A possible cause of seeing higher values with Stabilguard seem to be the enhancement of antibody-antigen (sHLA) interaction, increasing the antibody's affinity to its target during the ELISA procedure. Stabilguard is a possible candidate to be used in reactions of HLA with allosera. (The optimal % of Stabilguard needs to be established first).

Using agents such as PVP (FIG. 72) or PEG (FIG. 73) also showed good results. Known as crowding agents, they push proteins out of solutions in the mechanical/physical sense and in the thermodynamic sense. The crowding action, aided by any degree of affinity of protein molecules for one another promote protein-to-protein association. Conformationally loose protein molecules are ™Squeezed™ on by these agents, promoting protein molecule tightening and sometimes promoting an ordered protein conformation. Thus, these are the most potential candidates to be used in solution. In addition, 2% BSA and 10% FBS also worked, however with lesser intensity. The results obtained from 10% FBS compared to PBS also explains results earlier observed in the ELISA procedure in that ELISA values tend to be higher when crude harvest was tested than after purification testing the pure protein. It also explains why column efficiencies of only 60-70% were obtained since the efficiency is evaluated by the ratio of purified sHLA (measured in PBS) divided by the amount of sHLA loaded onto the column (measured in crude harvest containing 10% FBS).

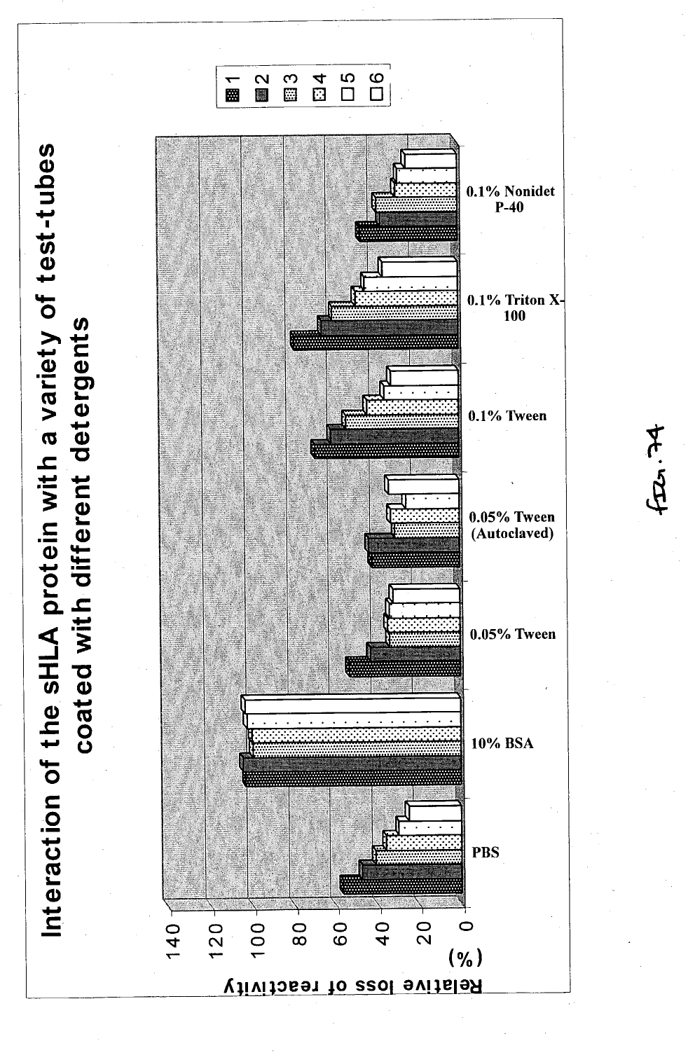

Finally, nonionic detergents did not greatly help preventing the loss of sHLA compared to 10% BSA (FIG. 74). However, these agents should not be excluded to be considered as supportive compounds since many proteins retain their activity in 1-3%. In the study presented here a 10 times lower concentration was used, and the trend of better performance can be seen (FIG. 74). , where 0.1% Tween 20 performed better than 0.05%.

In the above experiments, BSA, Stabilguard (StG), PEG and PVP were identified as potential blockers and/or stabilizers. However, the usage of the right concentration is important in the optimization procedure. Thus, different concentrations of blockers were tested by using a sHLA standard curve with declining concentrations.

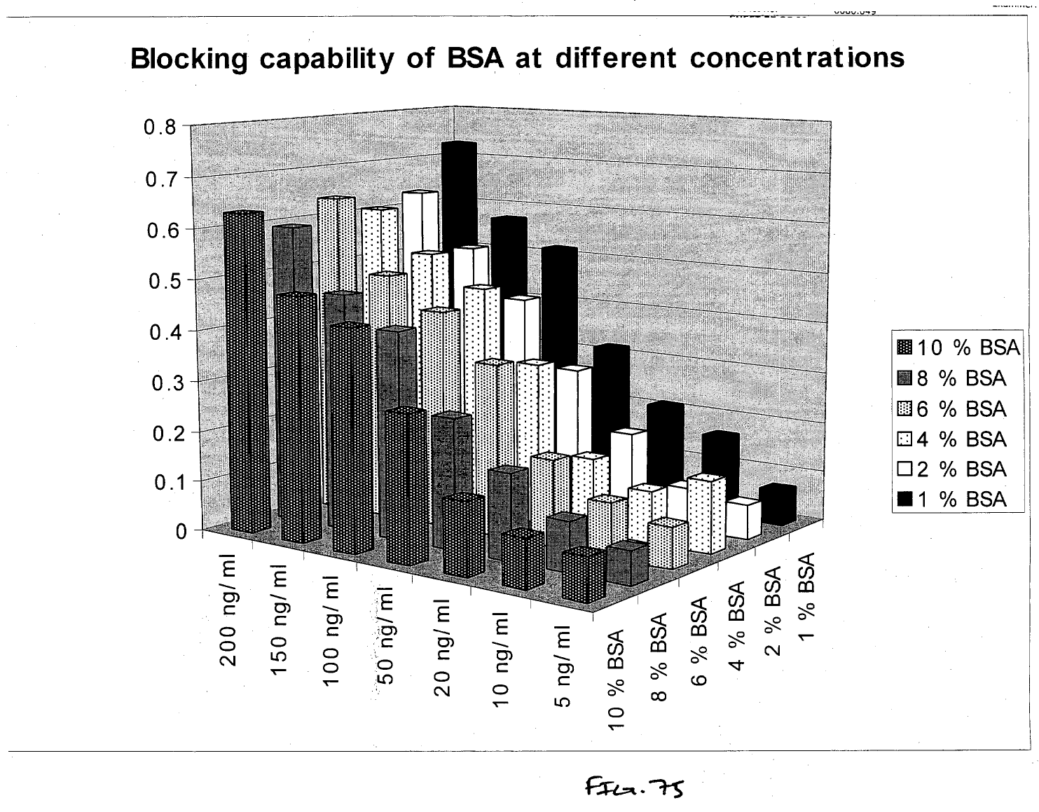

Concentrations of BSA between 2-10% do not show any difference in performance and are equally good (FIG. 75). The 1% BSA showed slightly higher values probably caused by an incorrect mixing of the stock solution. The results obtained with BSA suggest that the present usage of 10% BSA is not necessary and can be reduced to a lower percentage. The best choice is 3%, which will highly reduce the usage of chemicals and also buffer out minor mistakes in making the solution or helping to equalize dilution differences to a certain degree. Albumin did not interfere with the ability of serum and complement to lyse target cells. In standard lysis assay procedures, it was found that 30% albumin did not affect the ability of HLA antigens (Springer T A., JBC 1977; 4682-4693, the contents of which are hereby expressly incorporated by reference herein in their entirety.).

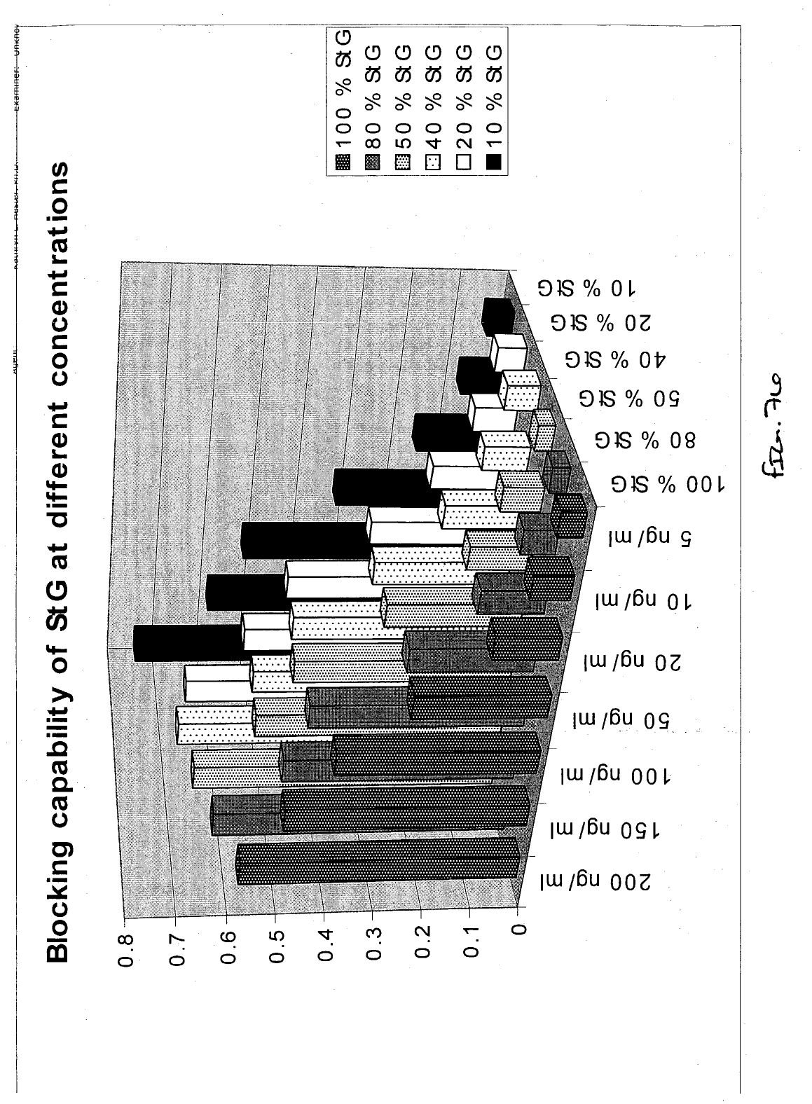

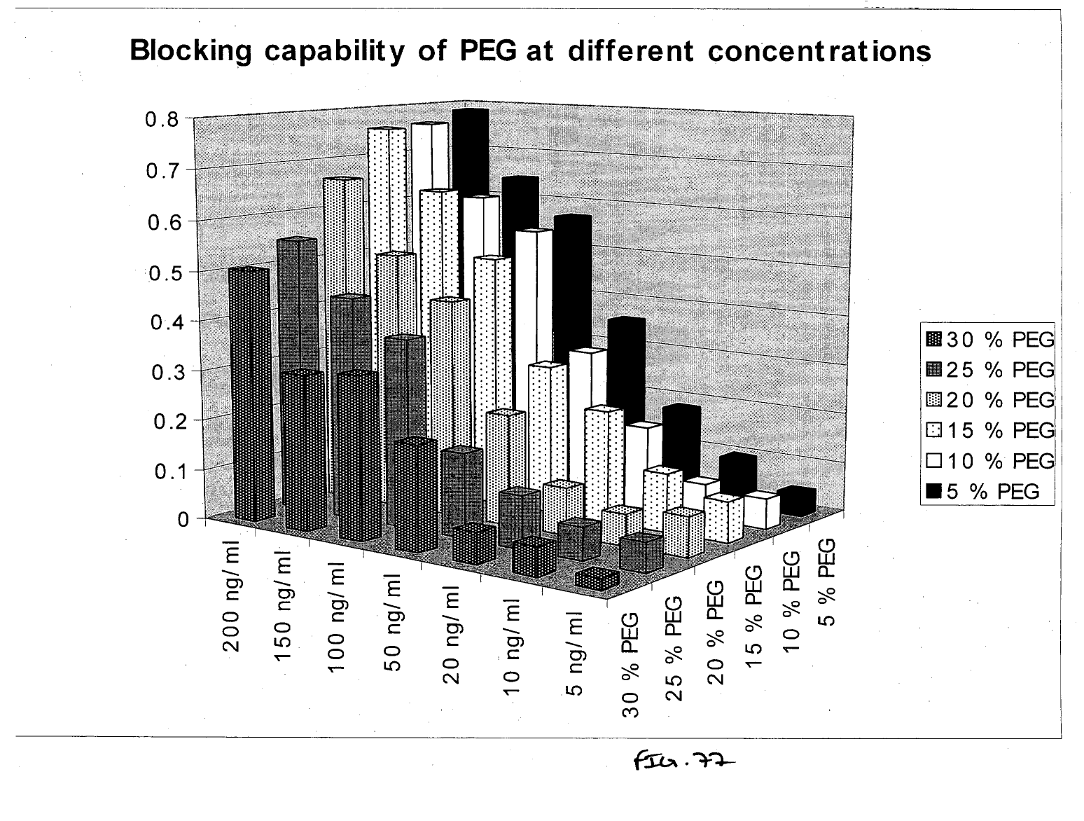

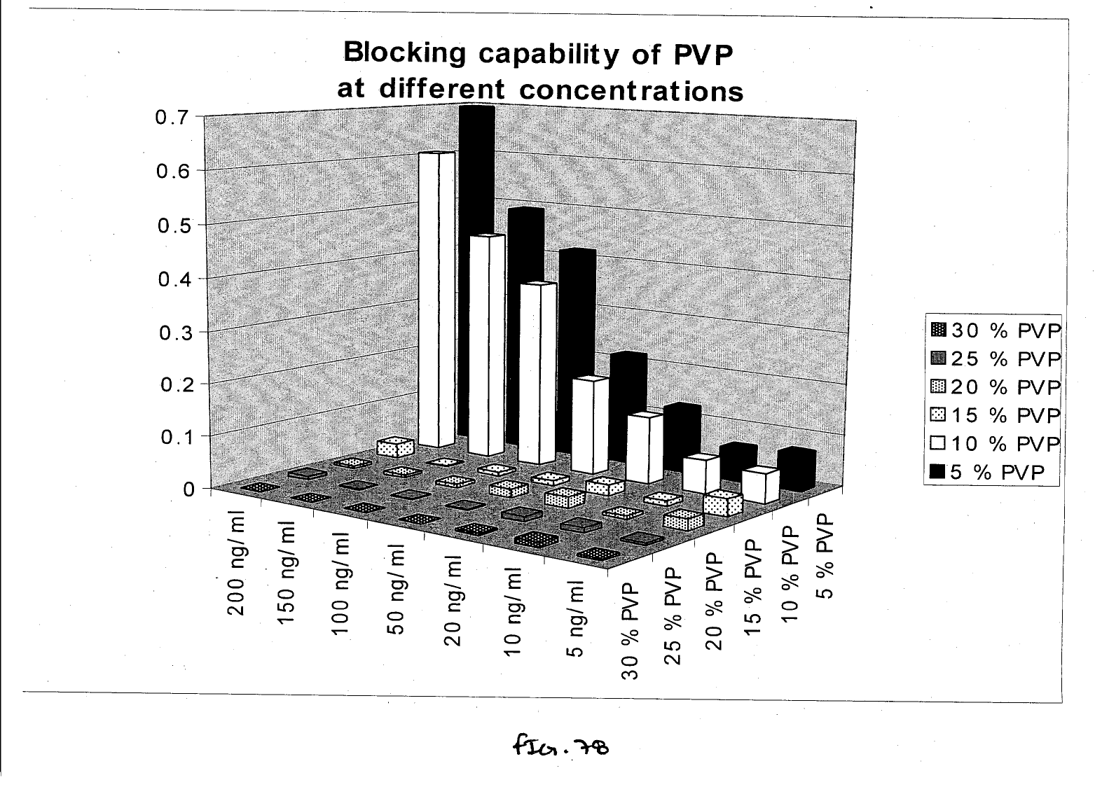

Stabilguard seems to work better with lower percentages (FIG. 76). A steady decrease in signal is observed using higher concentrated samples indicating an interference in protein-protein interaction rather then inefficiency in blocking. PEG can be used at concentrations up to 15% (FIG. 77). After that, PEG seems to highly interfere with the recognition of sHLA. PVP seems to be a great blocker at 5% (FIG. 78). However, it is absolutely not usable at higher concentrations, as it completely abolishes any interaction with sHLA.

Antigenic Integrity of sHLA for Use in Various Applications Sera Screen ELISA Prototype

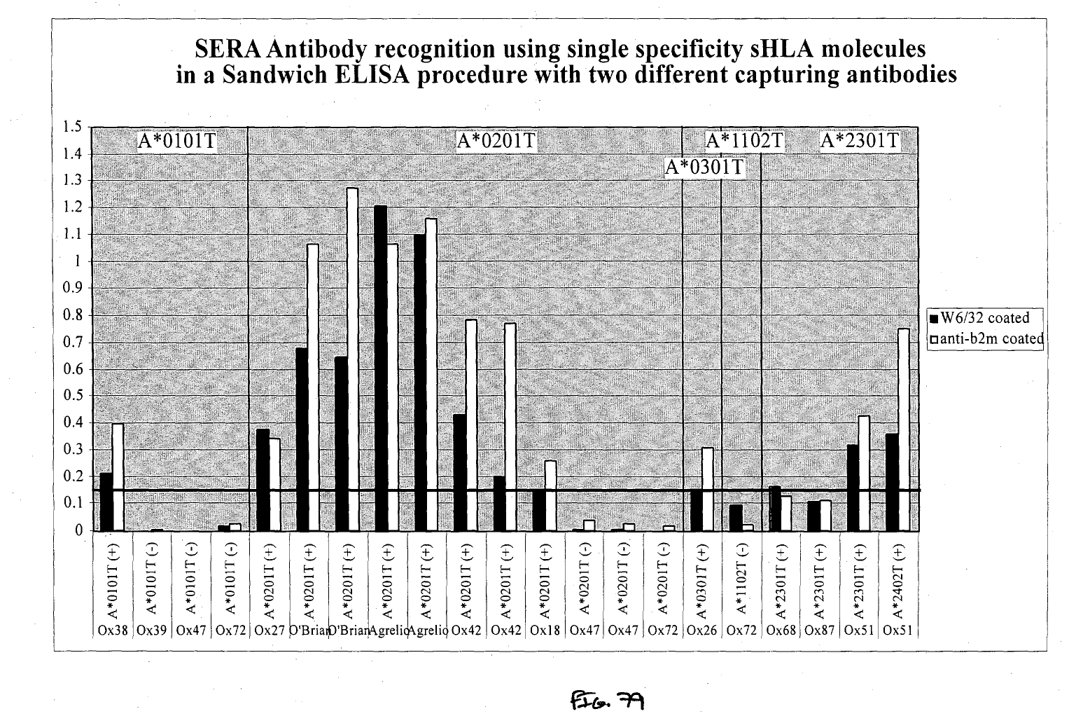

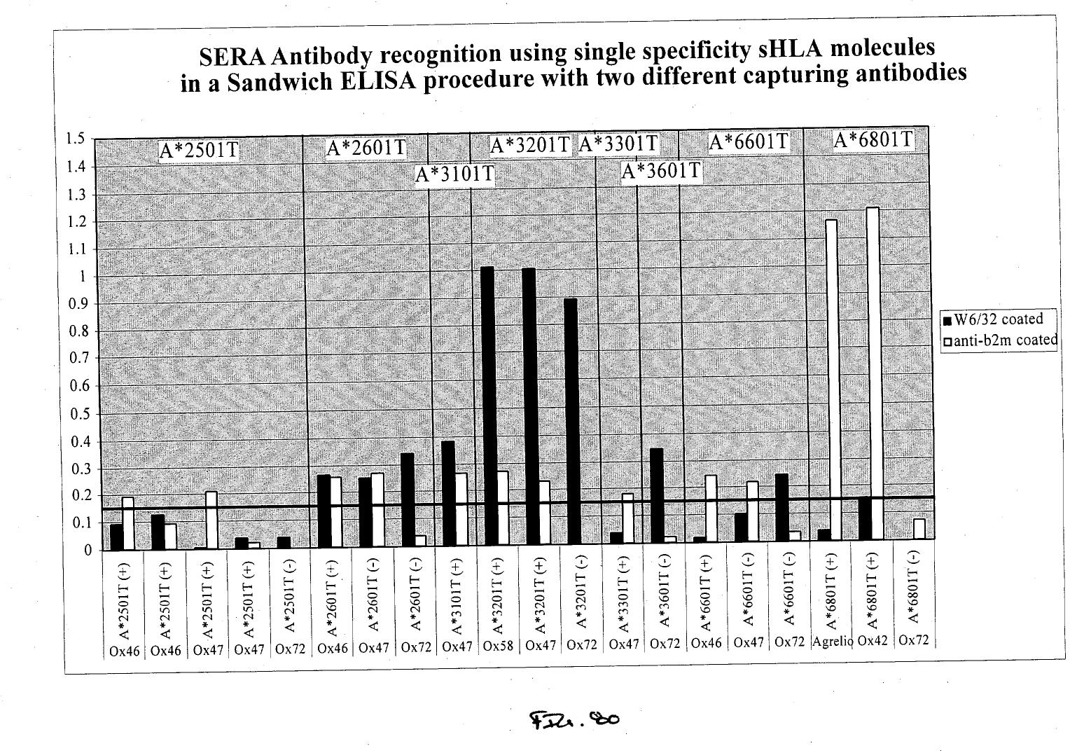

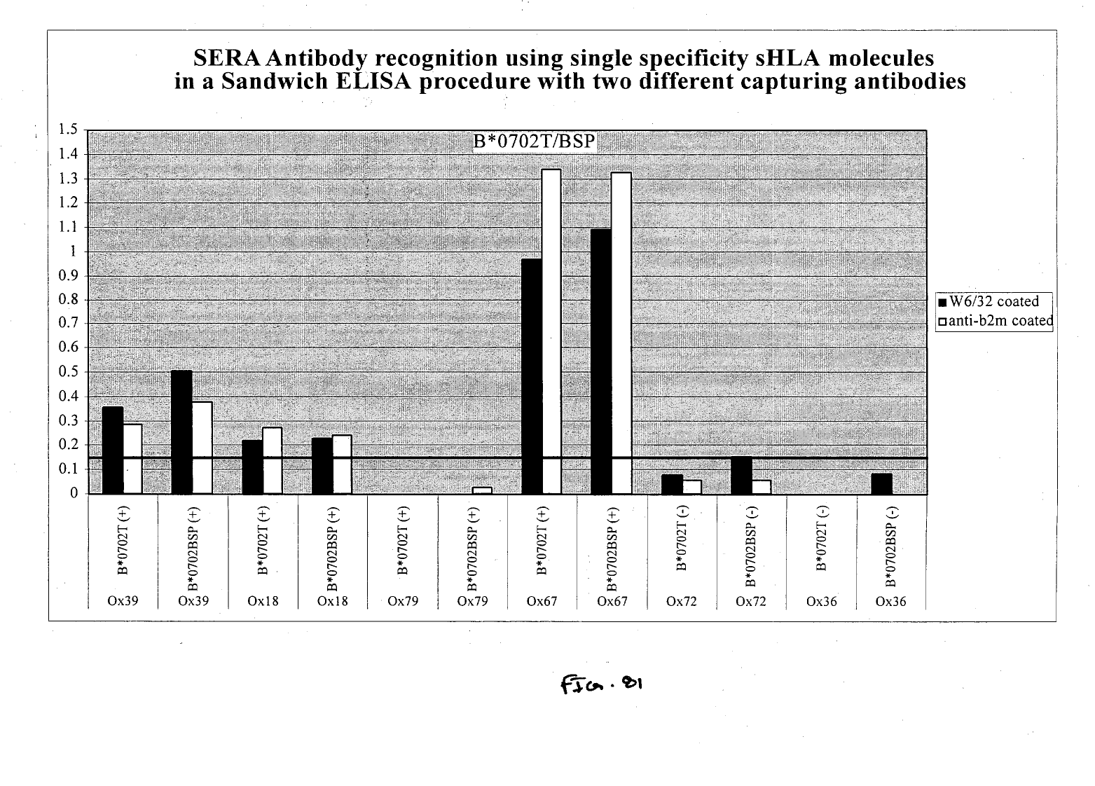

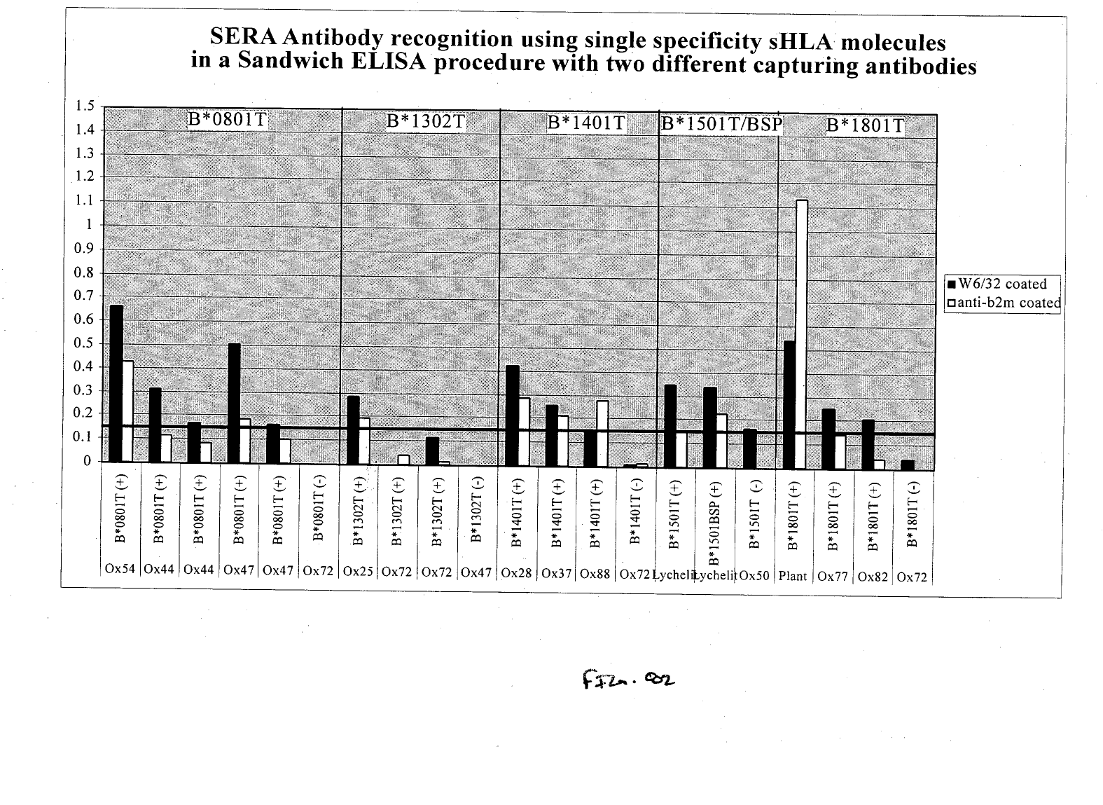

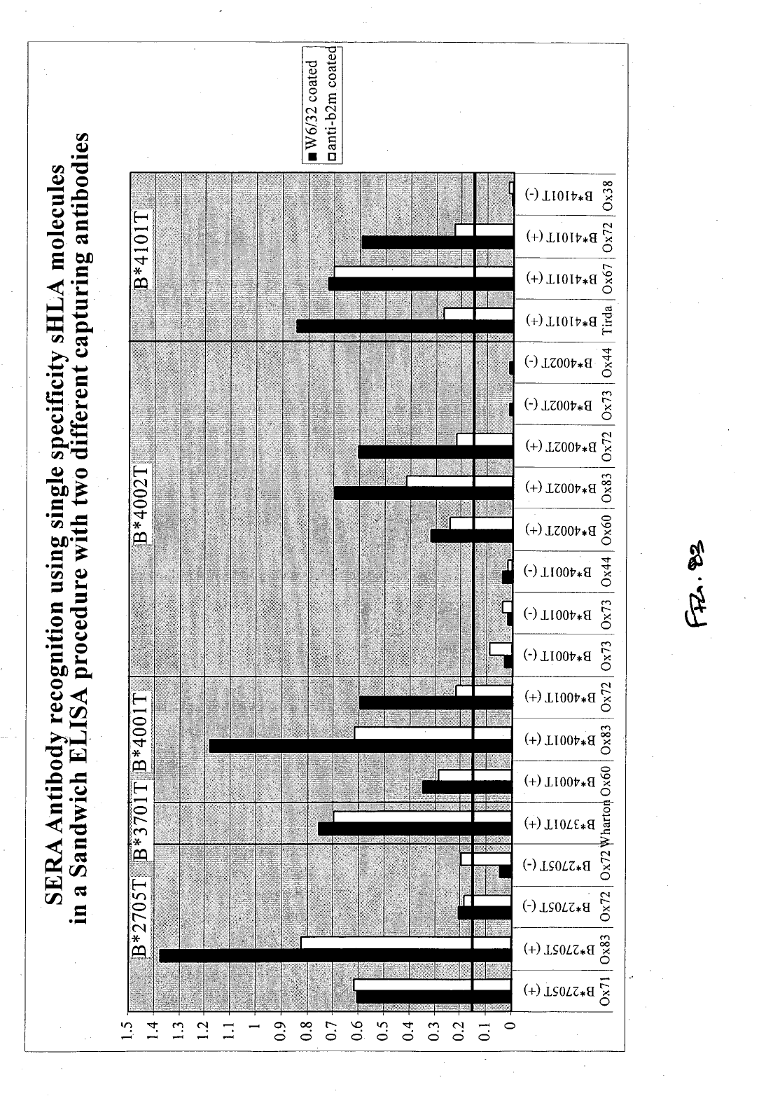

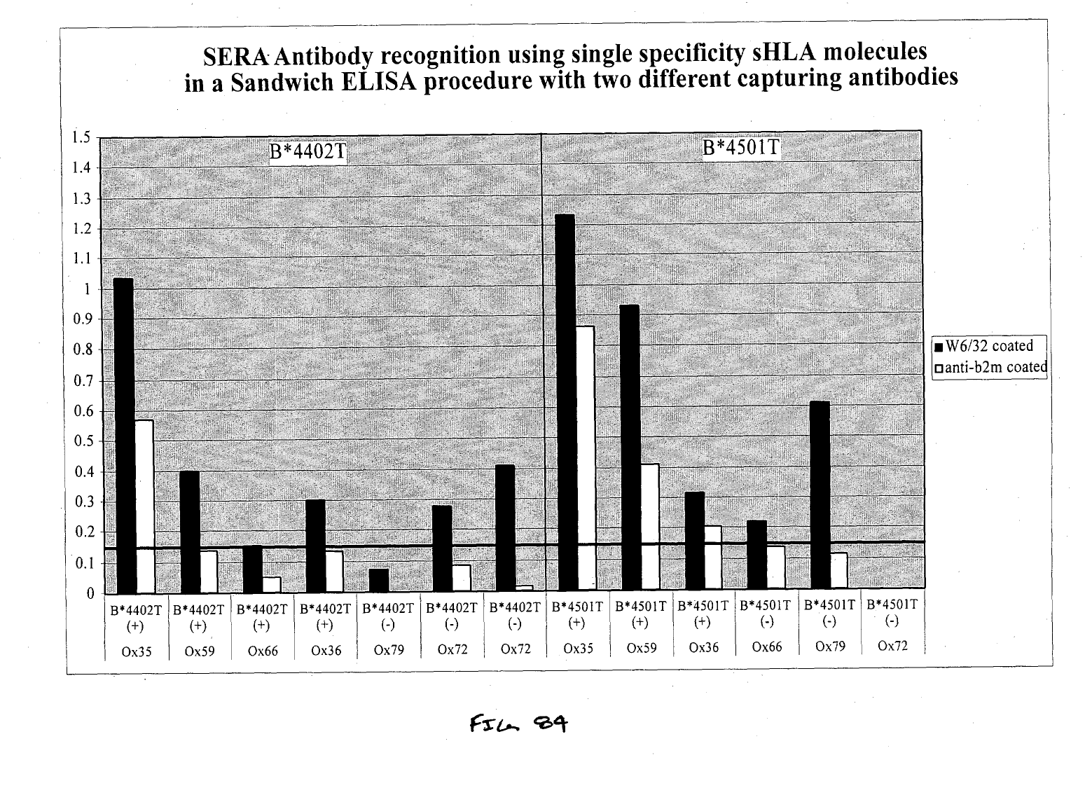

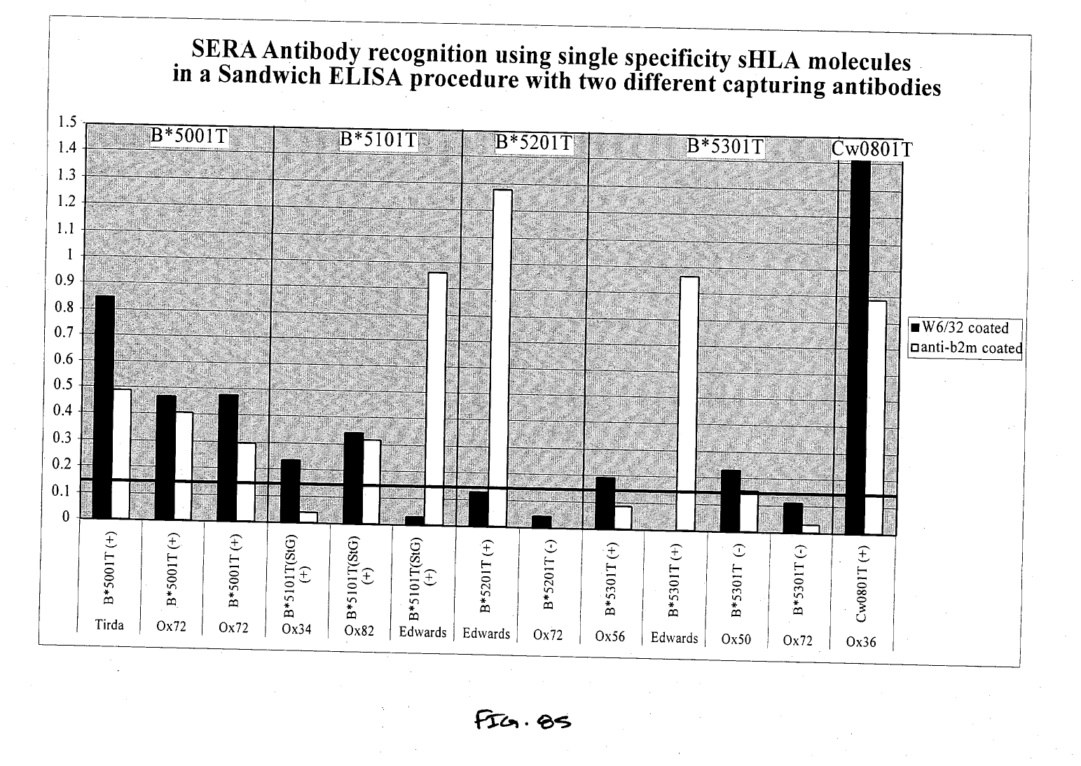

In the SERA SCREEN ELISA approach (described in detail in U.S. Serial No. 60/413,842, filed Sep. 24, 2002, the contents of which are hereby expressly incorporated herein by reference), the feasibility of a sera screen assay that utilizes HLA to identify antigen-specific antibodies in human sera was tested (FIGS. 79-85). The technique is based on an ELISA procedure utilizing W6/32 and anti-β2m as capturing antibody. These capturing antibodies present a panel of sHLA molecules at different orientations to guarantee the successful recognition by sera antibodies. In the final step, a secondary anti-human antibody coupled to HRP was used to visualize the positive sHLA-sera antibody interaction. All sHLA molecules used demonstrate reactivity with sera tested and thus prove the feasibility of this prototype.

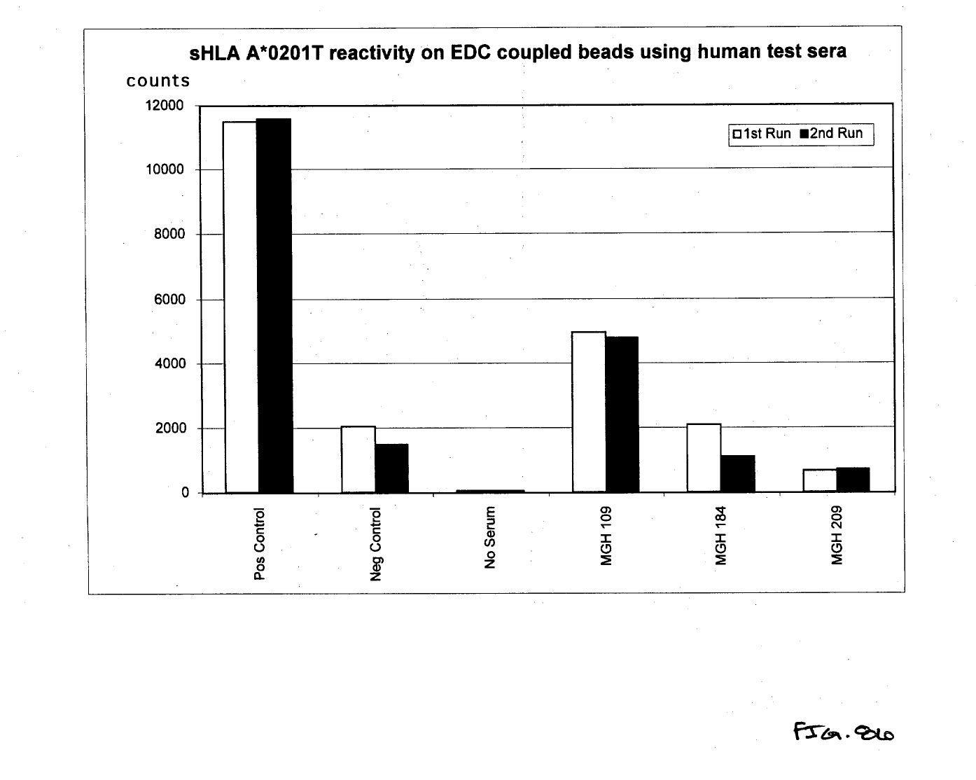

Coupling of sHLA molecules to Luminex™ beads to detect HLA antibodies in human sera can also be used with the individual, isolated, and purified sHLA molecules of the present invention. Disclosed herein is the information used to bind various sHLA alleles produced to a solid support in order to obtain specific recognition of the alleles by human sera. Binding to a solid bead support was accomplished via the EDC method, coupled sHLA to 1-ethyl-3-(3-dimethylaminoproplyl) carbodiimide-HCl (EDC) activated beads (FIG. 86). The results shown indicate that the isolated and purified sHLA of the present invention is indeed of high value in such assays.

Epitope Discovery

In this approach (described in detail in U.S. Serial No. 60/362,799, filed Mar. 7, 2002, the contents of which are hereby expressly incorporated herein by reference in their entirety), the feasibility of an assay that utilizes HLA technology in a high-throughput screening format to rapidly identify antigen-specific epitopes of infectious agents was tested. The proposed assay is based on competitive binding between a peptide of interest and a fluorescent-labeled standard peptide to a recombinant, soluble HLA (sHLA) molecule. Synthesized overlapping peptides covering any protein of interest can be screened for the ability to bind to their specific allele and their potential to stimulate immunoreactions. The state of the art fluorescence polarization (FP) methodology is utilized for monitoring binding in solution; the method offers an excellent assay format with respect to robustness, data quality and reproducibility. Equilibrium results obtained lead to an efficacious dose (IC₅₀), which is used to correlate in vitro potency of binding to the sHLA allele used in the assay. A sorting of IC₅₀ values into categories of high, medium, low, and no binding capability was used as the ultimate selection guide for the identification of potentially immunogenic peptides. Thus, the combination of sHLA technology with FP methodology will create a sensitive, highly reproducible, quantitative assay to measure the binding of defined synthetic antigenic peptides to various MHC class I alleles.

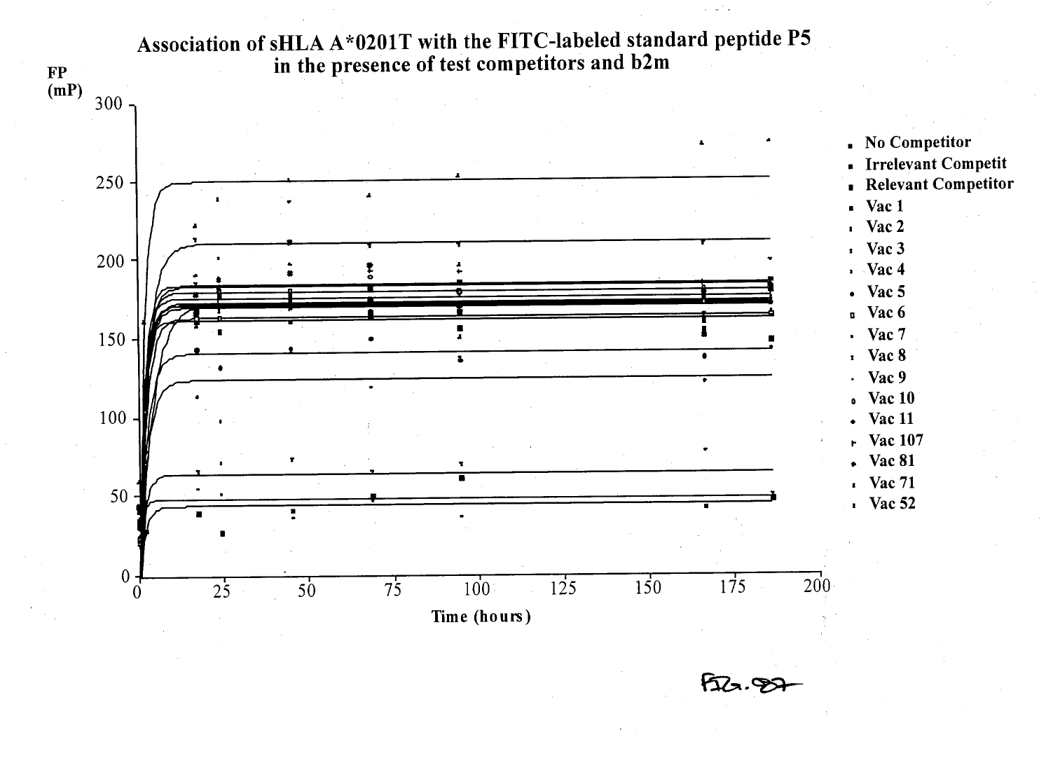

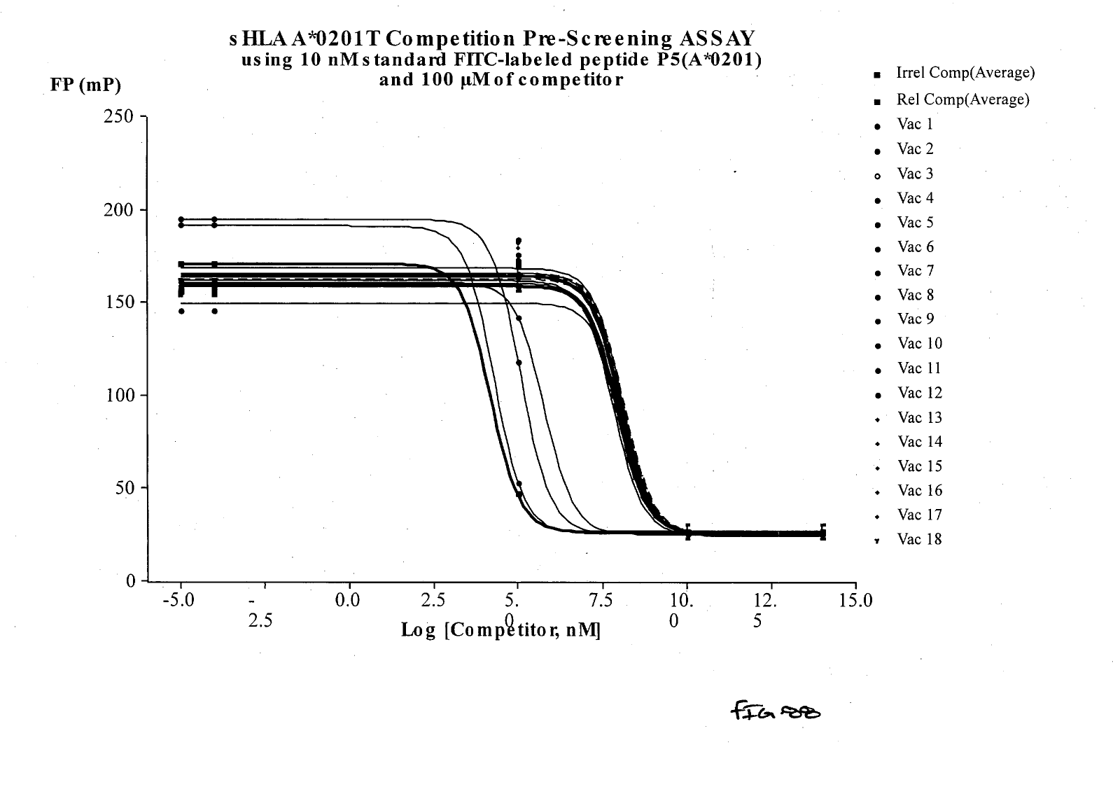

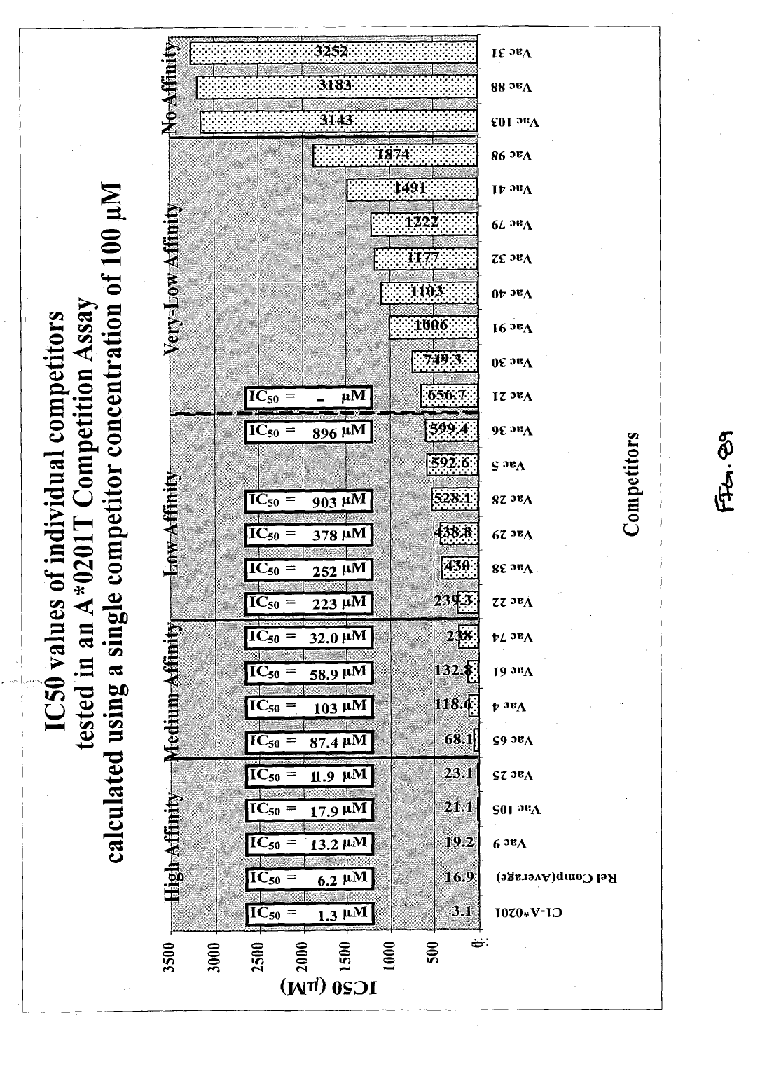

Test Competitors were pre-screened for their ability to inhibit a FITC-labeled standard peptide from binding to the sHLA molecule at a competitor concentration of 100 μM (FIG. 87). After obtaining equilibrium values for each test-peptide, IC50 values are calculated. A single measurement obtained at 100 μM competitor concentration can be used to construct such an IC50 value without support of additional data (FIG. 88). This constructed graph allows us to sort all competitors and easily categorize them into high, medium, low and no binders (FIG. 89). Additionally,. full scale IC50 determinations are performed on all candidates identified showing binding capacity to the allele tested. Usually, both methods are coming very close as seen in FIG. 89 in which one point IC50 determinations (bottom) are shown together with 8 point IC50 determinations (boxed, top).

Appropriate modification of the sequence of a peptide epitope can increase the affinity for the MHC molecule(s) without interfering with recognition by the TCR of T cells specific for the natural ligand sequence. Therefore, by this process of epitope enhancement or optimization, one should be able to create a more potent vaccine. The first step towards a successful epitope alteration approach is to increase the binding affinity and HLA-A2 stabilization capacity of HLA-A2-bound peptides. Since many immunodominant epitopes are high affinity MHC binders (Sette, 1994), one strategy is to increase the binding affinity of ‘intermediate to low’ binding peptides and therefore increase their potential as immunogens.

The second step is that these substitutions preserve the antigenic specificity and do not interfere with the peptide/TCR interaction. It is particularly noteworthy that the CTL responses raised against the modified peptide do cross-react with the naturally occurring epitope. This will depend upon the nature and position of the modification. Cross-recognition of native peptides and their modified variants by specific CTL is the most important issue in the design of optimized vaccines.

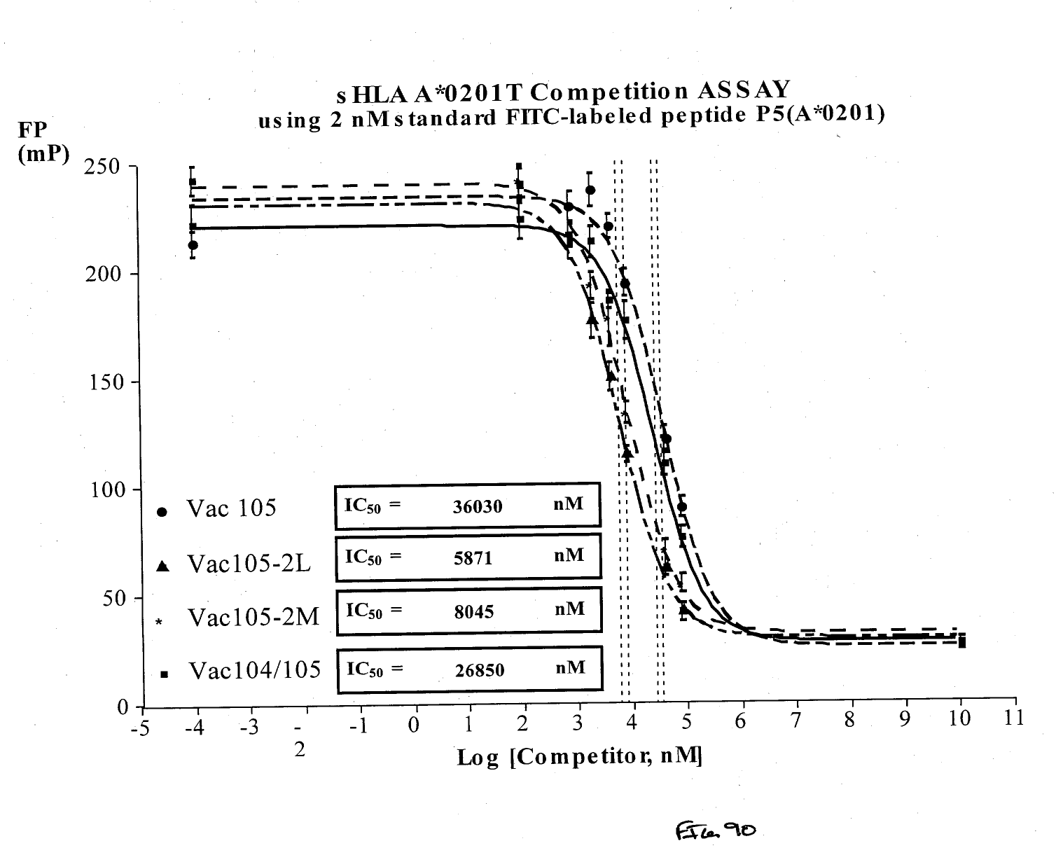

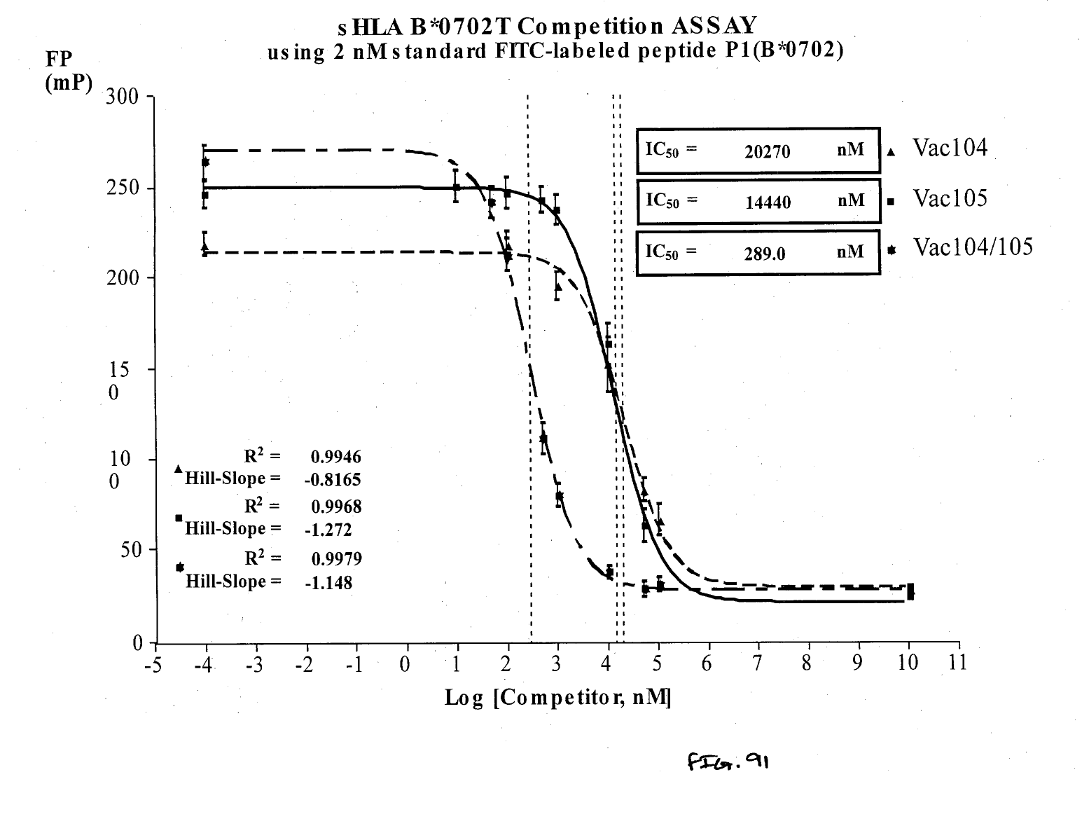

FIGS. 90 and 91 show improvement of modified peptides compared to the native test-peptide. FIG. 90 shows the IC50 of a native peptide Vac105 (ITNSRPPAV) to A*0201T whose binding capacity was improved by changing position 2T to 2L or 2M. The addition of an amino acid residue at the end did not result in a several fold improvement of binding (Vac104/105). FIG. 91 shows a much higher binding of the decamer Vac104/105 (KITNSRPPAV) than the two ninemers Vac104 (KITNSRPPA) or Vac105 (ITNSRPPAV).

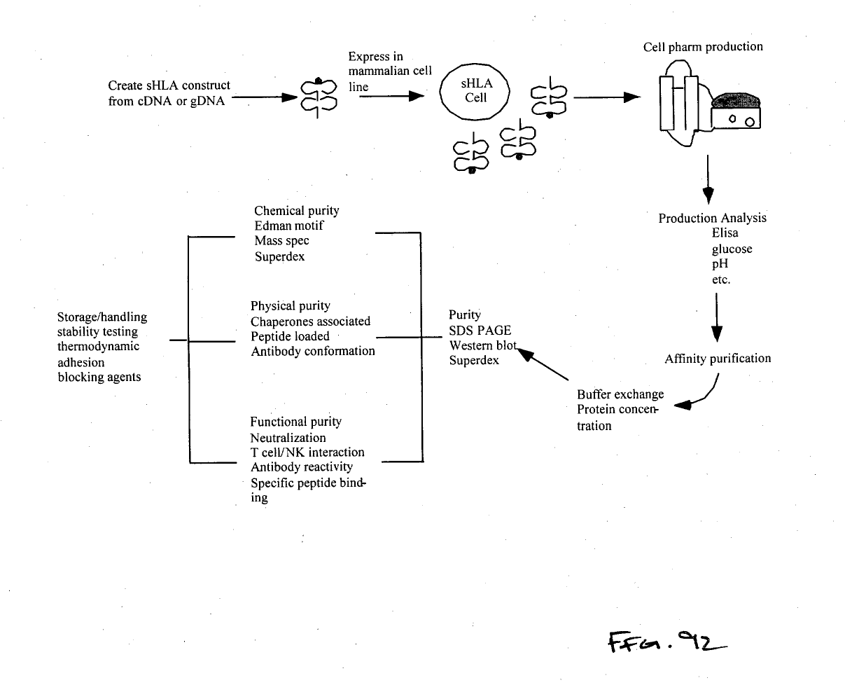

In summary, shown in FIG. 92 is a general outline of the purification and characterization procedures of soluble human HLA proteins of the present invention. The first step involves purification of soluble HLA, beginning with cell pharm run-large scale production of sHLA followed by production analysis. The sHLA is then purified by affinity column purification (which includes the steps of loading, washing and elution) and buffer exchange and concentration of purified allele using Macrocep concentration filters. The pure protein is then sterile filtered, aliquoted and stored, and the concentration of the stored pure protein is estimated. Finally, quality control demonstrating the extent of chemical purification is performed using techniques known to those of ordinary skill in the art, including but not limited to, SDS-PAGE, Western blot analysis, Superdex™ chromatography to demonstrate sample purity, and the like.

The second step in the method of the present invention involves characterization of the purified sHLA-peptide complex. Physical purity of the complex can be demonstrated by one or more of the following: sequence analysis to demonstrate the presence of all components of the complex; protein visualization procedures to demonstrate not only presence of all components but also formation of complex (including, but not limited to, SDS-PAGE, Western, Superdex™ chromatography, and the like); and Mass Spectrometry data for use in peptide motif comparisons. Functional purity of the complex can be demonstrated by one or more of the following: demonstration of antigenic integrity of sHLA using ELISA assays and neutralization experiments; demonstration of structural integrity using Chaperone interaction experiments; and demonstration of specificity, peptide binding capacity, and structural integrity using fluorescence polarization based association and saturation experiments.

The sHLA produced by the method of the present invention is feasible for use in the following various applications: sera screen assay that utilizes HLA to identify antigen-specific antibodies in human sera; Luminex bead approach to identify antigen-specific antibodies in human sera; competition assays, such as screening of test competitors for the ability to inhibit FITC-labeled standard peptide from binding to sHLA; and procedures to improve binding of modified peptides to sHLA as compared to native test-peptides. However, it is to be understood that many other applications for use of the sHLA produced by the purification method of the present invention will be evident to a person having ordinary skill in the art, and therefore the use of the sHLA produced by the purification method of the present invention is not limited to those listed above.

The final step in the method of the present invention involves determining the optimum storage and handling conditions for soluble HLA. The following factors in storage and handling have been described herein previously: stability testing in different buffers; thermodynamic stability of sHLA complexes; the influence of freeze-thaw cycles on stability; determination of loss of complex reactivity due to nonspecific adhesion to surfaces of storage vessels; and identification of appropriate blocking agents to maintain reactivity of sHLA.

Thus, in accordance with the present invention, there has been provided herein methods for the purification of soluble HLA, as well as characterization, storage and handling of the soluble HLA complex. FIG. 92 has provided a general outline that indicates how each of the individual experiments described herein previously are interrelated to each other in the methods of purification, characterization, storage and handling of the present invention.

Materials and Methods

Affinity Column Preparation

1. About 5-10 mg protein/ml swollen gel is recommended in coupling reactions in a volume of about 5 ml coupling buffer/g freeze-dried CNBr-activated Sepharose 4B. A carefully estimated ligand concentration is crucial in the success of the coupling reaction because of the ligand concentration dependence. Thus, dissolve the antibody or protein to be coupled in coupling buffer with a final concentration of 3.3-6.7 mg/ml.

| | | | | | | | | |
|---|---|---|---|---|---|---|---|---|
| Gel size (ml) | 1 | 2 | 3.5 | 5 | 10 | 50 | 100 | [conc.] |
| Coupling Buffer (ml) | 1.5 | 3 | 5.3 | 7.5 | 15 | 75 | 150 | (mg/ml) |
| Ligand (mg) | 2.5 | 5 | 8.8 | 12.5 | 25 | 125 | 250 | 1.66 |
| | 5 | 10 | 17.5 | 25 | 50 | 250 | 500 | 3.33 |
| | 6 | 12 | 21 | 30 | 60 | 300 | 600 | 4.00 |
| | 7 | 14 | 24.5 | 35 | 70 | 350 | 700 | 4.67 |
| | 8 | 16 | 28 | 40 | 80 | 400 | 800 | 5.33 |
| | 9 | 18 | 31.5 | 45 | 90 | 450 | 900 | 6.00 |
| | 10 | 20 | 35 | 50 | 100 | 500 | 1000 | 6.66 |
| | 12.5 | 25 | 43.8 | 62.5 | 125 | 625 | 1250 | 8.33 |
| | 15 | 30 | 52.5 | 70.5 | 150 | 750 | 1500 | 10 |

2. A very high ligand content can have three adverse effects on affinity chromatography. Firstly the binding efficiency of the adsorbent may be reduced due to steric hindrance between the active sites; this is particularly important when large molecules such as antibodies, antigens and enzymes are immobilized. Secondly, substances are more strongly bound to the immobilized ligand which may result in difficult elution. Thirdly, the extent of non-specific binding increases at very high ligand concentrations which can reduce the selectivity of the adsorbent.
3. Most advantageous is to dialyze the protein into coupling buffer the night before. Protein samples have to be up-concentrated if the mg/ml amount is to low for optimal coupling.

4. Calculate the proper dilution to match chosen protein concentration:

| | |
|---|---|
| Original concentration | c₁ = mg/ml |
| Chosen concentration | c₂ = mg/ml |
| Chosen final volume | V₂ = ml |
| Starting volume | V₁ = ml |

${V}_{1} = \frac{{c}_{2} {V}_{2}}{{c}_{1}}$

5. Before starting the coupling procedure, calibrate the spectrophotometer with coupling buffer and estimate the protein concentration at the beginning of the reaction. This value (start-value t_{S}) should be as accurate as possible to allow an estimation of the coupling efficiency (ligand binding efficiency). With the knowledge of total amount of antibody bound, a maximal antigen loading capacity can be calculated. However, this is only possible when the molecular weight of all interactive compounds is known. The reading is performed at A₂₈₀. Because stray light can affect the linearity of absorbance versus concentration, absorbance values >2.0 should not be used for any sample of proteins measured by the A₂₈₀ method.

6. To accurately convert A₂₈₀ to the actual antibody concentration use the following formula:

$\frac{{A}_{280} - {A}_{280} blank}{1.38} \times 1 mg / ml \times Dil . factor = mg / ml$

| | |
|---|---|
| start-value tₛ: | A₂₈₀ = ( ) mg/ml |
| Time: | 0 min |
| Dilutionfactor: | |

7. Weigh out the required amount of CNBr-activated Sepharose 4B. One g freeze-dried CNBr-activated Sepharose 4B swells to give approximately a 3.5 ml final gel volume. The active product is freeze-dried in the presence of dextran and lactose. Free cyanogen bromide is absent. (The freeze-dried material should be stored below 4° C. Under these conditions the shelf life is approximately 18 months, although further storage is not usually accompanied by rapid loss of activity. The opened package should be stored dry below 4° C.).

| | | | | | | | |
|---|---|---|---|---|---|---|---|
| Gel Size (ml) | 1 | 2 | 3.5 | 5 | 10 | 50 | 100 |
| Matrix (g) | 0.29 | 0.57 | 1.0 | 1.43 | 2.86 | 14.3 | 28.6 |

8. Coupling a ligand to the activated matrix involves first swelling and washing the gel in 1 mN HCl. The protein binding activity of the gel is preserved better by washing at low pH than by washing at pH's above 7. The use of HCl preserves the activity of the reactive groups which hydrolyze at high pH. Dextran and lactose, which are added to the activated gel to preserve its activity under freeze-drying, are washed away during the swelling stage.
9. Swelling and washing is performed in a sintered glass filter. A sintered glass filter is a glass funnel with a built-in glass frit. The glass frit is used instead of a membrane filter. The filter unit is placed on top of a side-arm vacuum flask and filtration occurs using suction/vacuum. The glass frit is available in different porosities. Medium porosity (porosity G3) is recommended for Sepharose.
10. Before starting to swell, clean the sintered glass filter with 0.5 N HCl and several rinses of ddH₂O. The final rinse should be done with 1 mN HCl.

11. The required amount of freeze-dried powder is suspended in 1 mN HCl. The gel swells immediately and should be washed during a time period of 15 minutes on the sintered glass filter with the same solution. Let the mixture equilibrate a few minutes during each washing step. Approximately 210 ml solution is added in several aliquots for each gram of dry gel. Suck off the supernatant between successive additions.

| | | | | | | | |
|---|---|---|---|---|---|---|---|
| Gel size (ml) | 1 | 2 | 3.5 | 5 | 10 | 50 | 100 |
| Matrix (g) | 0.29 | 0.57 | 1.0 | 1.43 | 2.86 | 14.3 | 28.6 |
| 1 mN HCl (ml) | 60 | 120 | 210 | 300 | 600 | 3000 | 6000 |

12. In 50 or 100 ml gel applications, the amount of 1 mN HCl may be difficult to handle. Recent studies have shown, however, that by increasing the contact time between gel and HCl, the amount of 1 mN HCl required to wash out these additives can be reduced to one third of this recommendation, without affecting the coupling reaction.
13. The final aliquot of 1 mN HCl is sucked off until cracks appear in the gel cake. Be sure swelling and washing is performed immediately before ligand coupling because activated groups hydrolyze in aqueous solutions and coupling capacity begins to decrease. Thus, immediately transfer the swollen gel to a solution of the ligand without delay. At pH 3, coupling activity is lost slowly, whereas at pH 9 activity is lost fairly rapidly.
14. Optional: It is possible to quickly wash the gel with 5 gel volumes of coupling buffer. However, hydrolysis will start at the same moment and decrease the coupling efficiency.
15. Transfer the swollen gel into a 50 ml Falcon tube or a 250 ml bottle by scooping the gel out of the sintered glass filter into the reaction vessel. Add some 1 mN HCl to the sinter, apply vacuum and collect small residues of the swollen gel.

16. Immediately add the appropriate volume of protein solution to the gel. A gel:buffer ratio of 1:2 to 2:3 gives a suitable suspension for coupling. In this protocol we calculated volumes for a ratio of 2:3. Rinse the filter with a small volume of the same solution.

| | | | | | | | |
|---|---|---|---|---|---|---|---|
| Gel size (ml) | 1 | 2 | 3.5 | 5 | 10 | 50 | 100 |
| Matrix (g) | 0.29 | 0.57 | 1.0 | 1.43 | 2.86 | 14.3 | 28.6 |
| Protein Solution (ml) | 1.5 | 3 | 5.3 | 7.5 | 15 | 75 | 150 |

17. Cap the reaction vessel, and agitate the gel gently on a rocker. Do not use magnetic stirrers as they usually cause fragmentation of the gel beads.
18. Coupling occurs very fast under our chosen conditions, and is usually complete after 20-30 minutes at room temperature (20-25° C.). If cold temperatures are necessary, coupling can also be performed overnight at 4° C. The amount of protein which couples under a given set of conditions depends mainly on the ratio of protein to gel volume, the pH of the reaction and the protein itself as well as the duration and temperature of the reaction. A number of conditions can lead to poor coupling: low. ligand concentration, suboptimal pH, impure ligand, improperly prepared matrix, inaccessibility of ligand or improperly prepared buffers.
19. The coupling reaction may be conveniently followed by observing the decrease in the absorbance of the supernatant solution at 280 nm. Thus, remove samples at different times during coupling and assay the buffer for the presence of antibodies. Measure A₂₈₀ at intervals of about 5 minutes and collect these values as coupling-values t₁₋ₓ. Since the reaction-mechanism is very fast, the starting values are more important than the later ones.
20. Aliquots need to be centrifuged for 30 seconds at full speed before the measurement. (The actual time-point for t₁₋ₓ is directly before starting the centrifuge).

21. To bring the protein samples within the spectrometers accuracy range, dilute them with an appropriate amount of coupling buffer if necessary. (Absorbance values >2.0 should not be used). Don't forget to mention the dilution-factor.

$\frac{{A}_{280} - {A}_{280} blank}{1.38} \times 1 mg / ml \times Dil . factor = mg / ml$

| | | | |
|---|---|---|---|
| coupling-value t₁: | A₂₈₀ = ( ) mg/ml | Time: | Dilutionfactor: |
| coupling-value t₂: | A₂₈₀ = ( ) mg/ml | Time: | Dilutionfactor: |
| coupling-value t₃: | A₂₈₀ = ( ) mg/ml | Time: | Dilutionfactor: |
| coupling-value t₄: | A₂₈₀ = ( ) mg/ml | Time: | Dilutionfactor: |
| coupling-value t₅: | A₂₈₀ = ( ) mg/ml | Time: | Dilutionfactor: |
| coupling-value t₆: | A₂₈₀ = ( ) mg/ml | Time: | Dilutionfactor: |
| coupling-value t₇: | A₂₈₀ = ( ) mg/ml | Time: | Dilutionfactor: |

22. After coupling is complete, spin at low speed (700 rpm) for 5 minutes to separate excess protein from the gel. Remove the supernatant from the gel slurry and save it to determine protein concentration after the coupling step (end-value tₑ). (Check if pH is still 9.0).

$\frac{{A}_{280} - {A}_{280} blank}{1.38} \times 1 mg / ml \times Dil . factor = mg / ml$

| | | | |
|---|---|---|---|
| end-value tₑ: | A₂₈₀ = ( ) mg/ml | Time: | Dilutionfactor: |

23. The next step is to wash away the excess ligand with coupling buffer. Most efficient way to wash the gel is to use the sintered glass filter.

| | | | | | | | |
|---|---|---|---|---|---|---|---|
| Gel size (ml) | 1 | 2 | 3.5 | 5 | 10 | 50 | 100 |
| Coupling Buffer (ml) | >50 | >100 | >180 | >200 | >350 | >800 | >1500 |

24. Block remaining active groups by transfering the gel to a vessel with 15 gel volumes of 0.1 M Tris-HCl, pH 8.0. Shake in an Erlenmayer flask at 180 rpm at room temperature for 2 hours. (Alternatively, active groups can also be blocked using 0.2 mM glycine, pH 8.0 or 1 M ethanolamine, pH 8.0).

| | | | | | | | |
|---|---|---|---|---|---|---|---|
| Gel size (ml) | 1 | 2 | 3.5 | 5 | 10 | 50 | 100 |
| Blocking Buffer (ml) | 15 | 30 | 52.5 | 75 | 150 | 750 | 1500 |

25. After the blocking, pour the solution back onto the filter. Rinse the tube with blocking buffer to collect most of the coupled gel.

26. The final product is then washed alternately with 10 gel volumes of low pH wash buffer (0.1 M sodium acetate containing 0.5 M NaCl, pH 4.0) and high pH wash buffer (0.1 M Tris-HCl containing 0.5 M NaCl, pH 8.0) for 4 times. Thorough washing of the coupled product is necessary to remove traces of non-covalently adsorbed materials. The washing-cycle of low and high pH is essential for the best results. This procedure ensures that no free ligand remains ionically bound to the immobilized ligand. Let the mixture equilibrate a few minutes during each washing step.

| | | | | | | | |
|---|---|---|---|---|---|---|---|
| Gel size (ml) | 1 | 2 | 3.5 | 5 | 10 | 50 | 100 |
| Wash Buffers (ml) each wash | 10 | 20 | 36 | 50 | 100 | 500 | 1000 |

27. Finally, pass 15 gel volumes of PBS over the sintered glass filter.

| | | | | | | | |
|---|---|---|---|---|---|---|---|
| Gel size (ml) | 1 | 2 | 3.5 | 5 | 10 | 50 | 100 |
| PBS (ml) | 15 | 30 | 52.5 | 75 | 150 | 750 | 1500 |

28. Transfer the gel into 2.5 gel volumes of PBS containing 0.05% sodium azide. The protein-sepharose conjugate is now ready for packing into columns.

| | | | | | | | |
|---|---|---|---|---|---|---|---|
| Gel size (ml) | 1 | 2 | 3.5 | 5 | 10 | 50 | 100 |
| Storage Buffer (ml) | 2.5 | 5 | 9 | 12.5 | 25 | 125 | 250 |

29. Store at 4° C. The stability of the coupled gel is dependent on the attached ligand and storage might be limited.

30. Collect all A₂₈₀ measurements in the following data chart. This data collection will be used to graph the reaction curve and calculate efficiency of the coupling reaction (ligand binding efficiency) as well as the antigen loading capacity of the column. These values are particularly useful to be compared to later performed coupling reactions.

| | | | |
|---|---|---|---|
| start-value tₛ: | A₂₈₀ = ( ) mg/ml | Time: 0 min | Dilution Factor: |
| Coupling-value t₁: | A₂₈₀ = ( ) mg/ml | Time: | Dilution Factor: |
| Coupling-value t₂: | A₂₈₀ = ( ) mg/ml | Time: | Dilution Factor: |
| Coupling-value t₃: | A₂₈₀ = ( ) mg/ml | Time: | Dilution Factor: |
| Coupling-value t₄: | A₂₈₀ = ( ) mg/ml | Time: | Dilution Factor: |
| Coupling-value t₅: | A₂₈₀ = ( ) mg/ml | Time: | Dilution Factor: |
| Coupling-value t₆: | A₂₈₀ = ( ) mg/ml | Time: | Dilution Factor: |
| Coupling-value t₇: | A₂₈₀ = ( ) mg/ml | Time: | Dilution Factor: |
| end-value tₑ: | A₂₈₀ = ( ) mg/ml | Time: | Dilution Factor: |

31. To estimate coupling efficiency (ligand binding efficiency), determine the concentration of the ligand in solution before and after the coupling step. Generally, 70-80% binding is optimal: lower binding leads to reduced column capacity while higher binding may result in reduced binding efficiency due to steric hindrance. Coupling efficiencies of 70-80% are normally a good compromise between good activity and high concentrations.

$$\frac{{[ conc . ]}_{{t}_{s}} - {[ conc . ]}_{{t}_{e}}}{{[ conc . ]}_{{t}_{s}}} 100 \% = \%$$
32. Calculate the total amount of antibody bound per ml of gel.

$$\frac{{( amount protein )}_{{t}_{s}} coupling efficiency}{ml gel} = ( mg \text{/} ml gel )$$
33. The total amount of antibody bound per ml of gel is directly proportional to the antigen loading capacity which will give an estimate of how much protein maximally can bind per ml gel. To take into consideration is the Mw of the IgG molecule of ˜150 kDa as well as its capability to bind 2 antigens. In addition, parameters of the molecule to purify are also necessary (i.e. class I complex (57 kDa): heavy chain; 45 kDa, β2-microglobulin; 12 kDa, peptide).

$$mg {IgG}_{bound} / ml gel \frac{57 kDa}{150 kDa} = mg {Antigen}_{max} / ml gel$$

A variety of columns are available for large scale purification. XK columns are jacketed and available in different dimensions with diameters of 26 mm (XK26) and 50 mm (XK50). These columns are only used with adaptors. The column can be used in aqueous and nearly all organic solvents (exceptions: acetone, chloroform, phenol). Solutions containing more than 10% NaOH, 10% HCl or 5% acetic acids should not be used. Kontes Flex-columns are a more simpler version of columns but as effective.
1. Sterilize the column before loading using either 100% ethanol or 2 N NaOH. It is possible to autoclave columns for 15 minutes at 121° C., wet or dry.
2. To start loading, resuspend the settled gel by gently mixing.
3. Degas using a vacuum aspirator.
4. Transfer the gel slurry into an appropriate column. Do not use acetone, benzyl-alcohol, chloroform, phenol, or dimethyl formaldahide because immediate damage will occur. The columns are resistant to acetic acid or NaOH.
5. Pack the column by pouring the gel into the vertically held column. Pour the slurry into the column in one continuous motion. Let the matrix settle by gravity flow until all slurry is transfered.
6. Insert the flow adapter into the packed column. First, purge the air from the flow adapter tubing and rinse the flow adapter.
7. Carefully insert the flow adaptor into the column until it touches the buffer. Avoid trapping air bubbles by slightly tipping the column, allowing the air to escape.
8. Slowly lower the flow adapter until it touches the top of the packed gel bed. The seal should be tight enough to allow the buffer to rise through the adapter instead of leaking around the seal. This will help clear trapped air in the adapter tubing.
9. Finally, completely seal the adapter against the column.
10. Equilibrate the column by passing 10 bed-volumes of PBS over the matrix.
11. The column is now packed and ready to use. How well the column is packed will have a major effect on the result of the separation.
12. Depending on the size of the column, different flow rates can be applied.
   ÄKTA™ Prime System for Standard Separation Applications

ÄKTA™ prime is a compact, automated liquid chromatography system. It is designed for standard separation applications. Flow rates up to 50 ml/min and pressures up to 1000 kPa can be applied. The system includes components for measuring UV, conductivity, generating gradients and collecting fractions. The ÄKTA™ prime system may be utilized in the large scale purification procedure of the present invention in accordance with manufacturer's recommendations.

Large Scale Purification Procedure

1. To start the chromatography procedure, prepare the ÄKTA™ prime system. The system can be used immediately but the spectrophotometers full ability will not be obtained until after 1 hour of lamp warm-up.
2. To prepare the system for a run, check that the buffer inlet tubings are immersed in the correct buffer vessels and the waste tubings are put into a waste bottle.
3. Only use degassed and filtered liquids to make sure that the liquid remains free from air bubbles. Degass by applying a vacuum to the solution.

4. Prepare and hook up the buffers necessary for an sHLA purification:

| | |
|---|---|
| 1. PBS, pH 7.4 | (Wash buffer) |
| 2. 20% Ethanol/70% Ethanol | (Cleaning solutions) |
| 3. 0.1 N NaOH | (MOK elution buffer) |
| 4. 50 mM Diethylamine (DEA), pH 11.3 | (MOK elution buffer) |
| 5. Protein sample | (The line is stored in PBS/0.05% |
| | Na Azide, pH 7.4) |
| 6. 0.2 N Acetic acid, pH ˜ 2.7 | (Cleaning & MOK solution) |
| 7. 0.1 M Glycine, pH 11.0 | (sHLA elution buffer) |

5. It is important to purge the lines after a new hook-up with about 50 ml of liquid to get the air out of the system. Purging can be done manually through the inlets of the buffer valve (A1-A8), while carefully immersing the tubing in the respective liquid.
6. To remove any trapped air bubbles in the flow path, purge the pump in the order PBS/20% ethanol/PBS/final buffer solution.
7. Next, prepare the recorder to monitor the purification. Autozero the built-in UV spectrophotometer with PBS as reference.
8. Equilibrate all material to the temperature at which the chromatography will be performed. For large scale purifications, attach the column entrance/exit to the system.
9. Equilibrate the column by passing 10 bed-volumes of PBS over the matrix.

10. Before starting any column purification, the protein concentration in the sample solution should be determined using a quantitative ELISA procedure. The sample volume loaded will depend on the size and loading capacity of the column and the concentration of the sample. Calculate the volume of the sample solution maximally saturating the column according to the columns capacity to bind the antigen.

| | |
|---|---|
| (A) Antigen concentration: | mg/ml antigen |
| (B) Antigen binding capacity: | mg antigen/ml gel |
| (C) Matrix volume: | ml gel |
| (D) Maximal amount of antigen: (B*C) | mg |
| (E) Sample volume: (D/A) | ml |

11. Since the binding capacity of the column will realistically not be reached, a much lower volume of sample solution should be chosen. A value between 40 to 50% of the calculated volume is more accurate which also will not result in the waste of lots of unbound antibody within the flow-through.

12. Prepare the antibody sample solution for purification. Spin crude harvest at 5,000 rpm for 25 minutes (JA10 rotor) to remove lipid and cell debris. The antigen solution must be free of particulate matter. Pour the supernatant into a suitable container. Prevent air bubble formation.

| | |
|---|---|
| Name of the crude harvest: | |
| Volume used: | ml |
| Amount of sample: | mg |

13. The simplest method to bind the antigen to the antibody/Sepharose 4B matrix is to apply the sample through the system pump and pass the protein solution down the column.

14. Set appropriate parameters to record the loading conditions on the recorder.

| | Chart Speed | Conductivity | Optical Density |
|---|---|---|---|
| Load | 0.1 mm/min | 0.5 V | 1.0 V |

15. Save a 1 ml probe from the starting material (LOAD) before the purification procedure for analysis purposes.
16. Set the buffer valve to position 5 and the injection valve to position LOAD. Make sure the inlet tubing is purged with sample buffer without any airbubbles present. To have a purged sample line, disconnect shortly the column before loading and circulate the sample within the system with higher flow rate.
17. Pass the solution slowly through the column with a flow rate of approximately 1.0 ml/min or lower to give the protein time to bind more efficiently. Higher flow rates will decrease efficiency. A disruption in flow may cause a rapid rise in back-pressure. If this occurs, immediately shut off the pump and check the gel bed for compression.
18. Collect the flow-through in an appropriate container. Keep until you are sure all material has bound to the column and negligible amounts are in the flow through. Take a sample at the end of the run (Ft) which should be analyzed.

19. Wash the column with PBS at 10 ml/min until UV absorbance at 280 nm is zero. For a large column use 2000-3000 ml wash buffer (PBS). Save the wash in a container until after the purification.

| | Chart Speed | Conductivity | Optical Density |
|---|---|---|---|
| Wash | 0.5 mm/min | 0.5 V | 1.0 V |

20. Prepare borosilicate collection tubes by adding 1.2 ml of 1 M Tris-HCl, pH 7.0 per 4.8 ml of fraction to be collected (1:4). Neutralization is a safety measure to preserve the activity of the eluted molecule.

21. Human MHC class I (SHLA) molecules are best eluted from a W6/32 column by 0.1 M glycine, pH 11.0. Absorbance is used for generating a protein elution profile.

| | Chart Speed | Conductivity | Optical Density |
|---|---|---|---|
| Elution | 0.5 mm/sec | 0.2 V | 0.1 V |

22. Place the collector arm over the first collection tube. Elute 4.8 ml per fraction at 10 ml/min. Immediately afterwards, mix each tube gently to bring the pH back to neutral. As with all protein solutions, avoid bubbling or frothing as this denatures the proteins. If a very low amount of protein is expected, change the conductivity on the recorder to a lower value.
23. Identify the antigen-containing fractions by absorbance at 280 nm on the chart and combine them during up-concentration.
24. Up-concentrate immediately and buffer exchange into PBS using MACROSEP™ centrifugal concentrators (Pall Filtron; Northborough, Mass.; MACROSEP 10K; OD010C37). Keep the protein on ice at all times and centrifuge at 4° C.
25. After the buffer exchange, prepare the sample for storage at 4° C. Filter the pure samples through a 0.2 μ filter and aliquot directly into sterile, screw cap tubes. Label appropriately.
26. Determine the absorbance at 280 nm as well as the protein concentration with the Micro BCA kit. Activity can be determined with a regular ELISA procedure.
27. The purity of the eluted sHLA can be assessed by SDS-PAGE, Western blotting or performing a Superdex column analysis.
28. After the elution, quickly re-equilibrate the column with PBS to avoid denaturation of the W6/32 antibody linked to it.
29. For analytical work in which more than one allele will be purified on the same column, extreme care must be taken. To be able to reuse the column, start a maintenance procedure after the reequilibration. Cleaning-in-place is a procedure, which removes contaminants such as lipids, precipitates or denatured proteins that may remain in the column after regeneration. Such contaminations are especially likely when working with crude materials. The procedure helps to maintain the capacity, flow properties and general performance.

30. Mock elute the column using buffers with alternating pH. Start running over 10 gel volumes of 0.2 N acetic acid followed by 10 gel volumes of 50 mM diethylamine, pH 11.3 at a speed of 10 ml/min. Repeat three times and always equilibrate with 10 gel volumes PBS between buffer changes.

| | Chart Speed | Conductivity | Optical Density |
|---|---|---|---|
| Mock-elution | 1.0 mm/min | 0.2 V | 0.1 V |

32. Sanitization inactivates microbial contaminants in the packed column and related equipment. One generally recommended procedure is to wash alternately with high and low pH buffers as performed in the coupling reaction.
33. For sanitization, disassemble the column and wash the matrix alternately with low pH wash buffer (0.1 M sodium acetate containing 0.5 M NaCl, pH 4.0) and high pH wash buffer (0.1 M Tris-HCl containing 0.5 M NaCl, pH 8.0) for 3 times followed by re-equilibration with PBS.
34. Reassemble the cleaned and sterilized column and store it at 4° C. in PBS containing 0.05% sodium azide.
35. After the column is removed, the ÄKTA™ prime system has to be cleaned carefully. Start with the cleaning of line 5, where the sample was hooked up. Rinse the system pump and include the fraction collector line.
36. First clean the inlet tubing, by manually running the system pump and flushing with 0.2 N acetic acid at 30 ml/min followed by 0.1 N NaOH. Always equilibrate with PBS. Don't forget to add a line between the injection valve and the UV detector as a bridge, as replacement of the column.
37. Finally, rinse with 20% ethanol. If the column was sanitized because of bacterial contamination, rinse with 70% ethanol.
   Buffer Exchange and Concentrating Samples Using Pal-Filtron Concentrators

MACROSEP™ centrifugal concentrators (Pall Filtron; Northborough, Mass.; MACROSEP 10K; OD010C37) provide rapid and convenient concentration, purification, and desalting of 5 ml to 15 ml biological samples. A starting sample of 15 ml can be concentrated to 0.5 ml in 30 to 60 minutes without multiple decanting steps. The MACROSEP's ease of use saves valuable lab time.

Each centrifugal concentrator is constructed of polypropylene and contains a low-protein-binding OMEGA™ membrane, two factors which significantly reduce non-specific adsorption and enable the device to yield the highest recoveries. OMEGA membranes are made from polyethersulfone (PES) specifically modified to minimize protein binding. These membranes provide equivalent or higher recoveries than comparable regenerated cellulose membranes. MACROSEP centrifugal devices are ideal for concentrating small peptides, oligonucleotides, nucleic acids, enzymes, antibodies, microbes, and other macromolecules.

Centrifugation up to 5,000×g provides the driving force for filtration, moving sample towards the encapsulated OMEGA membrane. Biomolecules larger than the nominal molecular weight cutoff of the membrane are retained in the sample reservoir. Solvent and low molecular weight molecules pass through the membrane into the filtrate receiver. The MACROSEP centrifugal concentrator is available with 9 different molecular weight cutoffs (MWCO): 1K, 3K, 10K, 30K, 50K, 100K, 300K, 1000K, and 0.3 μm. For maximum retention, select a MACROSEP device with a molecular weight cutoff that is 3 to 5 times smaller than the weight of the molecule to be retained.

For purification of sHLA molecules of the present invention, a 10K MACROSEP™ centrifugal concentrator is utilized in accordance with manufacturer's recommendations.
1. Insert the paddle firmly into the bottom of the sample reservoir of the 10K MACROSEP™ centrifugal concentrator (Pall Filtron; Northborough, Mass.; OD010C37). The ™hooks™ on the top part of the paddle must rest firmly in the notches on top of the sample reservoir. For best alignment, turn the reservoir upside down on the bench top and gently press the paddle into place. Attach the filtrate receiver to the bottom of the sample reservoir.
2. Pre-Rinsing (Optional): OMEGA™ membranes in the MACROSEP devices contain trace amounts of glycerine and sodium azide. If these chemicals interfere with an assay, they may be removed. Filter 15 ml of deionized water or buffer through the membrane.
3. Start to up-concentrate immediately with the low peak fractions first. (With some micro-concentrators, adsorption of protein to the walls of the unit as well as to the filter itself can be significant when the sample is very dilute).
4. Pipette up to 15 ml of sample (protein-eluate in neutralization buffer) from the fraction-collector glass-tube into the non-membrane side of the sample reservoir(s) using a 10 ml pipette. (Do not decant the samples as it will result in a higher loss).
5. Do not overfill. Place the cap on the reservoir.
6. Place the device(s) into a swinging bucket rotor. (In a fixed-angle rotor, align the MACROSEP so that one of the ™hooks™ faces the center of the centrifuge rotor. This prevents a buildup of macromolecules on the membrane paddle and allows the device's deadstop to function properly. A swinging-bucket rotor is self-aligning).
7. Always counterbalance the rotor.
8. Keep the protein on ice at all times and centrifuge at 4° C. A non-refrigerated micro-centrifuge may develop temperatures detrimental to protein samples when operated for extended periods; therefore it is usually best to have the non-refrigerated micro-centrifuge in a refrigerator or cold room for this operation, even though the filtration rate is reduced by the cold.
9. Spin at 3,500 rpm (1,000-5,000 g) at 4° C., typically for 30 to 60 minutes, to achieve the desired concentrate volume.
10. For desalting and/or buffer exchange, concentrate the sample at least tenfold.
11. After the spin, remove the filtrate from the collector and save it in an appropriately labeled 500 ml bottle. Keep the bottle on ice at all times.
12. Refill the same macrosep(s) and repeat the procedure until all fractions are up-concentrated.
13. In parallel to the up-concentration process, centrifuge the empty fraction collector tubes to recover remaining traces of protein sample. Add the recovered material to the macrosep(s).
14. After up-concentration, proceed with the buffer exchange by adding fresh exchange buffer of the desired composition.
15. Add exchange buffer (PBS/0.02% Na Azide) to the sample reservoir in a volume equal or lower to that of the ultrafiltrate collected, so that the concentration of macromolecular species remains unchanged.
16. As filtration proceeds, refill the sample reservoir with fresh exchange buffer to restore the original volume. Continue doing this until the volume of ultrafiltrate is four times the volume of the original sample, indicating that removal of diffusible material is 95% to 99% complete.
17. After every fresh buffer exchange, make a mark on the top of the reservoir cap. This will help keeping track of the status of the procedure.
18. If there is not enough time to finish the whole procedure, it can be stopped after 2 buffer exchanges. Refill the macrocep with exchange buffer to prevent the membrane from going dry, put the cap on and store at 4° C. until the next day. Thereafter, the procedure can be interrupted any time, but always prevent the membrane from going dry by filling the reservoir.
19. Recombine the buffer exchange flow through with the original filtrate. Keep on ice.
20. After the buffer exchange, the same process is used to concentrate samples, except that the retentate volume is allowed to decrease until the desired degree of concentration is reached. Over-concentration makes sample recovery difficult and may require re-addition of buffer to wash the membrane, thereby adding to the volume.
21. Check OD₂₈₀ to estimate an approximate concentration of the sample. An OD₂₈₀ of 1.0 is in the area of 0.5 to 0.7 mg/ml.
22. To recover the final sample, remove the liquid from the sample reservoir with a 1000 μl pipette tip. Add to a labeled 50 ml Falcon tube and store at 4° C.
23. In regard to the recovery rate of samples following concentration being generally 95% and the degree of nonspecific adsorption of protein to membranes, losses of 5% to 10% are not uncommon when dealing with total quantities of protein in the range of 1 to 10 mg.
24. To recover with a much higher efficiency, add all the saved filtrate and flowthroughs again to the same macrocep(s) and proceed in the same way. Do not save filtrates a second time. Buffer exchange again four times and finally combine with the first round concentrate. Make sure to reach an equal concentration before combining.
25. For maximum concentrate recovery, remove filtrate receiver and screw on the concentrate cup. The center pin will cause the paddle to lift up and out of the bottom of the sample reservoir, allowing concentrate to flow into concentrate cup.
26. Place the MACROSEP device back into the centrifuge and spin at 3,500 rpm (1,000-5,000 g) for 5 minutes. Remove the device and unscrew the concentrate cup.
27. Finally, prepare the sample for storage at 4° C. Filter the pure sample through a 0.2 μm filter and aliquot directly into sterile, screw cap tubes. Label appropriately.
   ELISA Procedures

1. The experiment is designed using an ELISA protocol template, and a clear 96-well polystyrene assay plate is labeled. Polystyrene is normally used as a microtiter plate. (Because it is not translucent, enzyme assays that will be quantitated by a plate reader should be performed in polystyrene and not PVC plates).

| Company | Plate | Specificity | Cat# |
|---|---|---|---|
| Nunc | Maxisorp | standard/untreated | 441653 |
| | StarWell Modules | | |
| | Framed 8-well | | |
| | strips | | |

2. Coating of the W6/32 should be performed in Tris buffered saline (TBS); pH 8.5. Prepare a coating solution of 8.0 μg/ml of specific W6/32 antibody in TBS (pH 8.5). (Use the blue tube preparation stored at −20° C. a concentration of 0.2 mg/ml and a volume of 1 ml giving 0.2 mg per tube).

| | No. of plates | Total Volume | W6/32 antibody | TBS, pH 8.5 |
|---|---|---|---|---|
| Mix: | 1 | 10 ml | 400 μl | 9.6 ml |
| | 2 | 20 ml | 800 μl | 19.2 ml |
| | 3 | 30 ml | 1200 μl | 28.8 ml |
| | 4 | 40 ml | 1600 μl | 38.4 ml |
| | 5 | 50 ml | 2000 μl | 48.0 ml |

3. Although this is well above the capacity of a microtiter plate, the binding will occur more rapidly. Higher concentrations will speed the binding of antigen to the polystyrene but the capacity of the plastic is only about 100 ng/well (300 ng/cm²), so the extra protein will not bind.
4. If using W6/32 of unknown composition or concentration, first titrate the amount of standard-antibody solution needed to coat the plate versus a fixed, high concentration of labeled antigen. Plot the values and select the lowest level that will yield a strong signal.
5. Do not include sodium azide in any solutions when horseradish peroxidase is used for detection.
6. Immediately coat the microtiter plate with 100 μl per well using a multi-channel pipette. Standard polystyrene will bind antibodies or antigens when the proteins are simply incubated with the plastic. The bonds that hold the proteins are non-covalent, but the exact types of interactions are not known.
7. Shake the plate to ensure that the antigen solution is evenly distributed over the bottom of each well.
8. Seal the plate with plate sealers (sealplate adhesive sealing film, nonsterile, 100 per unit; Phenix (1-800 767-0665); LMT-Seal-EX) or sealing tape to Nunc-Immuno™ Modules (# 236366).
9. Incubate at 4° C. overnight. Avoid detergents and extraneous proteins.
10. Next day, remove the contents of the well by flicking the liquid into the sink or a suitable waste container. Remove the last traces of solution by inverting the plate and blotting it against clean paper. toweling. Complete removal of liquid at each step is essential for good performance.
11. Wash the plate 10 times with Wash Buffer (PBS containing 0.05% Tween-20) using a multi-channel ELISA washer.
12. After the last wash, remove any remaining Wash Buffer by inverting the plate and blotting it against clean paper toweling.
13. After the W6/32 is bound, the remaining sites on the plate must be saturated by incubating with blocking buffer made of 3% BSA in PBS. Fill the wells with 200 μl blocking buffer.
14. Cover the plates with an adhesive strip and incubate overnight at 4° C. Alternatively, incubate for at least 2 hours at room temperature which is, however, not the standard procedure.
15. Blocked plates may be stored for at least 5 days at 4° C.
16. Good pipetting practice is most important to produce reliable quantitative results. The tips are just as important a part of the system as the pipette itself. If they are of inferior quality or do not fit exactly, even the best pipette cannot produce satisfactory results.
17. The pipette working position is always vertical: Non-vertical positions may cause too much liquid to be drawn in.
18. The immersion depth should be only a few millimeters.
19. Allow the pipetting button to retract gradually, observing the filling operation. There should be no turbulence developed in the tip, otherwise there is a risk of aerosols being formed and gases coming out of solution.
20. When maximum levels of accuracy are stipulated, pre-wetting should be used at all times. To do this, the required set volume is first drawn in one or two times using the same tip and then returned. Pre-wetting is absolutely necessary on the more difficult liquids such as 3% BSA.
21. Do not pre-wet if your intention is to mix your pipetted sample thoroughly with an already present solution.
22. However, pre-wet only for volumes greater than 10 μl. In the case of pipettes for volumes less than 10 μl, the residual liquid film is as a rule taken into account when designing and adjusting the instrument. The tips must be changed between each individual sample.
23. With volumes <10 μl special attention must also be paid to drawing in the liquid slowly, otherwise the sample will be significantly warmed up by the frictional heat generated. Then slowly withdraw the tip from the liquid, if necessary wiping off any drops clinging to the outside.
24. To dispense the set volume hold the tip at a slight angle, press it down uniformly as far as the first stop.
25. In order to reduce the effects of surface tension, the tip should be in contact with the side of the container when the liquid is dispensed.
26. After liquid has been discharged with the metering stroke, a short pause is made to enable the liquid running down the inside of the tip to collect at its lower end.
27. Then press it down swiftly to the second stop, in order to blow out the tip with the extended stroke with which the residual liquid can be blown out. In cases that are not problematic (e.g. aqueous solutions) this brings about a rapid and virtually complete discharge of the set volume. In more difficult cases, a slower discharge and a longer pause before actuating the extended stroke can help.
28. To determine the absolute amount of antigen (sHLA), sample values are compared with those obtained using known amounts of pure unlabeled antigen in a standard curve.
29. For accurate quantitation, all samples have to be run in triplicate, and the standard antigen-dilution series should be included on each plate. Pipetting should be preformed without delay to minimize differences in time of incubation between samples.
30. All dilutions should be done in blocking buffer.
31. Thus, prepare a standard antigen-dilution series by successive dilutions of the homologous antigen stock in 3% BSA in PBS blocking buffer. In order to measure the amount of antigen in a test sample, the standard antigen-dilution series needs to span most of the dynamic range of binding. This range spans from 5 to 100 ng sHLA/ml.
32. A stock solution of 1 μg/ml should be prepared, aliquoted in volumes of 300 μl and stored at 4° C. Prepare a 50 ml batch of standard at the time. (New batches need to be compared to the old batch before used in quantitation).
33. Use a tube of the standard stock solution to prepare successive dilutions according to the scheme shown in FIG. 93.
34. While standard curves are necessary to accurately measure the amount of antigen in test samples, they are unnecessary for qualitative ™yes/no™ answers.
35. For accurate quantitation, the test solutions containing sHLA should be assayed over a number of at least 4 dilutions to assure to be within the range of the standard curve. Prepare serial dilutions of each antigen test solution in blocking buffer (3% BSA in PBS).
36. Standard dilutions for purified, crude or flow through samples are given in FIG. 94.
37. After mixing, prepare all dilutions in disposable U-bottom 96 well microtiter plates before adding them to the W6/32-coated plates with a multipipette. Add 150 μl in each well.
38. Next remove any remaining blocking buffer and wash the plate as described above. The plates are now ready for sample addition.
39. Add 100 μl of the sHLA containing test solutions and the standard antigen dilutions to the antibody-coated wells.
40. Cover the plates with an adhesive strip and incubate for exactly 1 hour at room temperature.
41. After incubation, remove the unbound antigen by washing the plate 10× with Wash Buffer (PBS containing 0.05% Tween-20) as described.

42. Prepare the appropriate developing reagent to detect sHLA. Use the second specific antibody, anti-human β2m-HRP (DAKO P0174/0.4 mg/ml) conjugated to Horseradish Peroxidase (HRP). Dilute the anti-human β2m-HRP in a ratio of 1:1,000 in 3% BSA in PBS. (Do not include sodium azide in solutions when horseradish peroxidase is used for detection).

| | No. of plates | Total Volume | Anti-β2m-HRP antibody | 3% BSA in PBS |
|---|---|---|---|---|
| Mix: | 1 | 10 ml | 10 μl | 10 ml |
| | 2 | 20 ml | 20 μl | 20 ml |
| | 3 | 30 ml | 30 μl | 30 ml |
| | 4 | 40 ml | 40 μl | 40 ml |
| | 5 | 50 ml | 50 μl | 50 ml |

43. Add 100 μl of the secondary antibody dilution to each well. All dilutions should be done in blocking buffer.
44. Cover with a new adhesive strip and incubate for 20 minutes at room temperature.
45. Prepare the appropriate amount of substrate prior to the wash step. Bring the substrate to room temperature.
46. OPD (o-Phenylenediamine) is a peroxidase substrate suitable for use in ELISA procedures. The substrate produces a soluble end product that is yellow in color. The OPD reaction is stopped with 3 N H₂SO₄, producing an orange-brown product and read at 492 nm. Prepare OPD fresh from tablets (Sigma, P6787; 2 mg/tablet). The solid tablets are convenient to use when small quantities of the substrate are required.
47. After second antibody incubation, remove the unbound secondary reagent by washing the plate 10× with Wash Buffer (PBS containing 0.05% Tween-20).
48. After the final wash, add 100 μl of the OPD substrate solution to each well and allow it to develop at room temperature for 10 minutes. Reagents of the developing system are light-sensitive, thus, avoid placing the plate in direct light.
49. Prepare the 3 N H₂SO₄ stop solution.
50. After 10 minutes, add 100 μl of stop solution per 100 μl of reaction mixture to each well. Gently tap the plate to ensure thorough mixing.
51. Read the ELISA plate at a wavelength of 490 nm within a time period of 15 minutes after stopping the reaction.
52. The background should be around 0.1. If the background is higher, the substrate may have been contaminated with a peroxidase. If the subtrate background is low and the background in you're the assay is high, this may be due to insufficient blocking.
53. Finally analyze the readings.
54. Prepare a standard curve constructed from the data produced by serial dilutions of the standard antigen.
55. To determine the absolute amount of antigen, compare these values with those obtained from the standard curve. Use the pre-made Excel template.
   Protein Separation

SDS-PAGE

To localize sHLA with SDS-PAGE, proteins were obtained by denaturating with a solution containing 4% SDS, 20% glycerol, 0.02% bromophenol blue, and 200 mM dithiothreitol in 0.5 M Tris-HCl (pH 6.8). For separation, Sodium dodecyl sulfate-polyacrylamide gel elec-trophoresis (SDS-PAGE) was performed by using the procedures described previously by [Laemmli, 1970] on a 12.5% gel. Gels were stained in Coomassie-staining.

Western Blot Analysis

To localize sHLA in Western blots, proteins were obtained by denaturating with a solution containing 4% SDS, 20% glycerol, 0.02% bromophenol blue, and 200 mM dithiothreitol in 0.5 M Tris-HCl (pH 6.8). Sodium dodecyl sulfate-polyacrylamide gel elec-trophoresis (SDS-PAGE) was performed by using the procedures described previously by [Laemmli, 1970]. Briefly, the proteins were separated on a 12.5% gel, electroblotted onto an Immobilon-P membranes (Millipore, Bedford, Mass.), and blocked overnight in 3% BSA in Tris-buffered saline/ Tween 20 buffer. All primary and secondary antibodies were applied in this buffer. The working dilution of primary antibodies was 1:1,000 for β2m(HRP), and 1:5000 for HC10, and that of horseradish peroxidase (HRP)-conjugated goat anti-mouse IgG antibody was 1:2,000. To visualize antibody binding, the membranes were developed using the ECLplus reaction according to the manufacturer's recommendation.

Thus, in accordance with the present invention, there has been provided a method for purifying Class I and Class II MHC molecules substantially away from other proteins that includes methodology for producing and manipulating Class I and Class II MHC molecules from gDNA that fully satisfies the objectives and advantages set forth herein above. Although the invention has been described in conjunction with the specific drawings, experimentation, results and language set forth herein above, it is evident that many alternatives, modifications, and variations will be apparent to those skilled in the art. Accordingly, it is intended to embrace all such alternatives, modifications and variations that fall within the spirit and broad scope of the invention.

### References

The following references, to the extent that they provide exemplary procedural or other details supplementary to those set forth herein, are specifically incorporated herein by reference in their entirety as though set forth herein particular.
Cresswell, P., M. J. Turner, and J. L. Strominger, Papain-solubilized HL-A antigens from cultured human lymphocytes contain two peptide fragments. Proc Natl Acad Sci U S A, 1973. 70(5): p. 1603-7.
Tanigaki, N. and D. Pressman, The basic structure and the antigenic characteristics of HL-A antigens. Transplant Rev, 1974. 21(0): p. 15-34.
Tanigaki, N., et al., Common antigenic structures of HL-A antigens. II. Small fragments derived from papain-solubilized HL-A antigen molecules. Immunology, 1974. 26(1): p. 155-68.
Prilliman, K., et al., Large-scale production of class I bound peptides: assigning a signature to HLA-B*1501. Immunogenetics, 1997. 45(6): p. 379-85.
Prilliman, K. R., et al., HLA-B15 peptide ligands are preferentially anchored at their C termini. J Immunol, 1999. 162(12): p. 7277-84.
Prilliman, K. R., et al., Peptide motif of the class I molecule HLA-B*1503. Immunogenetics, 1999. 49(2): p. 144-6.
Cresswell, P., et al., Papain-solubilized HL-A antigens. Chromatographic and electrophoretic studies of the two subunits from different specificities. J Biol Chem, 1974. 249(9): p. 2828-32.
Peterson, P. A., L. Rask, and J. B. Lindblom, Highly purified papain-solubilized HL-A antigens contain beta2-microglobulin. Proc Natl Acad Sci U S A, 1974. 71(1): p. 35-9.
Collins, E. J., et al., The three-dimensional structure of a class I major histocompatibility complex molecule missing the alpha 3 domain of the heavy chain. Proc Natl Acad Sci U S A, 1995. 92(4): p. 1218-21.
Bjorkman, P. J. and P. Parham, Structure, function, and diversity of class I major histocompatibility complex molecules. Annu Rev Biochem, 1990. 59: p. 253-88.
Laemmli, U. K et al., Cleavage of structural proteins during the assembly of the head of bacteriophage T4. Nature,1970, 227, p. 680-685.

What is claimed is:

1. A functionally active, individual soluble HLA molecule purified substantially away from other proteins such that the individual soluble HLA molecule maintains the physical, functional and antigenic integrity of the native HLA molecule.

2. The functionally active, individual soluble HLA molecule of claim 1 wherein the functionally active, individual soluble HLA molecule is purified by affinity chromatography and fractionation.

3. The functionally active, individual soluble HLA molecule of claim 2 wherein the affinity chromatography utilizes W6/32 antibodies or other pan-specific class I HLA molecules.

4. The functionally active, individual soluble HLA molecule of claim 1 wherein the functionally active, individual soluble HLA molecule is a Class I HLA molecule or a Class II HLA molecule.

5. The functionally active, individual soluble HLA molecule of claim 1 wherein the functionally active, individual soluble HLA molecule is further defined as having an endogenous peptide loaded therein.

6. A functionally active, individual soluble HLA molecule purified substantially away from other proteins such that the individual soluble HLA molecule maintains the physical, functional and antigenic integrity of the native HLA molecule, the functionally active, individual soluble HLA molecule produced by the method comprising the steps of:
isolating HLA allele mRNA from a source and reverse transcribing the mRNA to obtain allelic cDNA;
amplifying the allelic cDNA by PCR, wherein the amplification utilizes at least one locus-specific primer that truncates the allelic cDNA, thereby resulting in a truncated PCR product having the coding regions encoding cytoplasmic and transmembrane domains of the allelic cDNA removed such that the truncated PCR product has a coding region encoding a soluble HLA molecule;
inserting the truncated PCR product into a mammalian expression vector to form a plasmid containing the truncated PCR product having the coding region encoding a soluble HLA molecule;
electroporating the plasmid containing the truncated PCR product into at least one suitable host cell;
inoculating a cell pharm or a large scale mammalian tissue culture system with the at least one suitable host cell containing the plasmid containing the truncated PCR product such that the cell pharm produces soluble HLA molecules, wherein the soluble HLA molecules are folded naturally and are trafficked through the cell in such a way that they are identical in functional properties to an HLA molecule expressed from the HLA allele mRNA and thereby bind peptide ligands in an identical manner as full-length, cell-surface-expressed HLA molecules;
harvesting the soluble HLA molecules from the cell pharm or large scale tissue culture system; and
purifying the individual, soluble HLA molecules substantially away from other proteins, wherein the individual soluble HLA molecules maintain the physical, functional and antigenic integrity of the native HLA molecule.

7. The functionally active, individual soluble HLA molecule of claim 6 wherein the functionally active, individual soluble HLA molecule is a Class I HLA molecule or a Class II HLA molecule.

8. The functionally active, individual soluble HLA molecule of claim 6 wherein the functionally active, individual soluble HLA molecule is further defined as having an endogenous peptide loaded therein.

9. The functionally active, individual soluble HLA molecule of claim 6 wherein, in the step of isolating HLA allele mRNA from a source, the source is selected from the group consisting of mammalian DNA and an immortalized cell line.

10. The functionally active, individual soluble HLA molecule of claim 6 wherein, in the step of inserting the truncated PCR product into a mammalian expression vector, the mammalian expression vector contains a promoter that facilitates increased expression of the truncated PCR product.

11. The functionally active, individual soluble HLA molecule of claim 6 wherein, in the step of electroporating the plasmid containing the truncated PCR product into at least one suitable host cell, the suitable host cell lacks expression of Class I HLA molecules.

12. The functionally active, individual soluble HLA molecule of claim 6 wherein, in the step of amplifying the allelic cDNA by PCR, the locus-specific primer includes a sequence encoding a tail such that the soluble HLA molecule encoded by the truncated PCR product contains a tail attached thereto that facilitates in purification of the soluble HLA molecules produced therefrom.

13. The functionally active, individual soluble HLA molecule of claim 6 wherein, in the step of amplifying the allelic cDNA by PCR, the at least one locus-specific primer includes a stop codon incorporated into a 3′ primer.

14. The functionally active, individual soluble HLA molecule of claim 6 wherein, in the step of purifying the individual, soluble HLA molecules substantially away from other proteins, the functionally active, individual soluble HLA molecule is purified by affinity chromatography and fractionation.

15. The functionally active, individual soluble HLA molecule of claim 14 wherein the affinity chromatography utilizes W6/32 antibodies.

16. A functionally active, individual soluble HLA molecule purified substantially away from other proteins such that the individual soluble HLA molecule maintains the physical, functional and antigenic integrity of the native HLA molecule, the functionally active, individual soluble HLA molecule produced by the method comprising the steps of:
obtaining gDNA encoding a HLA allele;
amplifying the allelic gDNA by PCR, wherein the amplification utilizes at least one locus-specific primer that truncates the allelic gDNA, thereby resulting in a truncated PCR product having the coding regions encoding cytoplasmic and transmembrane domains of the allelic gDNA removed such that the truncated PCR product has a coding region encoding a soluble HLA molecule;
inserting the truncated PCR product into a mammalian expression vector to form a plasmid containing the truncated PCR product having the coding region encoding a soluble HLA molecule;
electroporating the plasmid containing the truncated PCR product into at least one suitable host cell;
inoculating a cell pharm with the at least one suitable host cell containing the plasmid containing the truncated PCR product such that the cell pharm produces soluble HLA molecules, wherein the soluble HLA molecules are folded naturally and are trafficked through the cell in such a way that they are identical in functional properties to an HLA molecule expressed from the HLA allele mRNA and thereby bind peptide ligands in an identical manner as full-length, cell-surface-expressed HLA molecules;
harvesting the soluble HLA molecules from the cell pharm; and
purifying the individual, soluble HLA molecules substantially away from other proteins, wherein the individual soluble HLA molecules maintain the physical, functional and antigenic integrity of the native HLA molecule.

17. The functionally active, individual soluble HLA molecule of claim 16 wherein the functionally active, individual soluble HLA molecule is a Class I HLA molecule or a Class II HLA molecule.

18. The functionally active, individual soluble HLA molecule of claim 16 wherein the functionally active, individual soluble HLA molecule is further defined as having an endogenous peptide loaded therein.

19. The functionally active, individual soluble HLA molecule of claim 16 wherein, in the step of obtaining gDNA which encodes a HLA allele, the gDNA is obtained from blood, saliva, hair, semen, or sweat.

20. The functionally active, individual soluble HLA molecule of claim 16 wherein, in the step of inserting the truncated PCR product into a mammalian expression vector, the mammalian expression vector contains a promoter that facilitates increased expression of the truncated PCR product.

21. The functionally active, individual soluble HLA molecule of claim 16 wherein, in the step of electroporating the plasmid containing the truncated PCR product into at least one suitable host cell, the suitable host cell lacks expression of Class I HLA molecules.

22. The functionally active, individual soluble HLA molecule of claim 16 wherein, in the step of amplifying the allelic cDNA by PCR, the locus-specific primer includes a sequence encoding a tail such that the soluble HLA molecule encoded by the truncated PCR product contains a tail attached thereto that facilitates in purification of the soluble HLA molecules produced therefrom.

23. The functionally active, individual soluble HLA molecule of claim 16 wherein, in the step of amplifying the allelic cDNA by PCR, the at least one locus-specific primer includes a stop codon incorporated into a 3′ primer.

24. The functionally active, individual soluble HLA molecule of claim 16 wherein, in the step of purifying the individual, soluble HLA molecules substantially away from other proteins, the functionally active, individual soluble HLA molecule is purified by affinity chromatography and fractionation.

25. The functionally active, individual soluble HLA molecule of claim 24 wherein the affinity chromatography utilizes W6/32 antibodies.
